(19) United States
(12) Patent Application Publication
(10) Pub. No.: US 2025/0270567 A1
(43) Pub. Date: Aug. 28, 2025

(54) GENERATION OF ACYL AMINO ACIDS

(71) Applicant: Modular Genetics, Inc., Lincolin, MA (US)

(72) Inventors: Kevin A. Jarrell, Lincoln, MA (US); Michelle Pynn, Andover, MA (US)

(21) Appl. No.: 18/883,913

(22) Filed: Sep. 12, 2024

Related U.S. Application Data
(63) Continuation of application No. 17/260,984, filed on Jan. 15, 2021, now Pat. No. 12,123,004.
(60) Provisional application No. 62/700,118, filed on Jul. 18, 2018.

(51) Int. Cl. C12N 15/52; C12N 1/20; C12N 9/02; C12N 9/10; C12N 9/90; C12P 13/14; C12R 1/125

(52) U.S. Cl. CPC C12N 15/52; C12N 1/20; C12N 9/0071; C12N 9/1007; C12N 9/90; C12P 13/14; C12R 2001/125; C12Y 502/01004

(57) ABSTRACT

Engineered polypeptides or engineered microbial cells useful in synthesizing acyl amino acids are provided. In some embodiments, engineered polypeptides or engineered microbial cells are useful in synthesizing acyl amino acids with one or more hydroxyl and/or methyl groups at one or more positions of the fatty acid portion of the acyl amino acid (e.g., at ω-1, ω-2, and/or ω-3 positions of the fatty acid portion of the acyl amino acid). Also provided are methods of making acyl amino acids using engineered polypeptides and/or engineered microbial cells.

### CROSS-REFERENCE TO RELATED APPLICATIONS

This application is a continuation of U.S. application Ser. No. 17/260,984 filed Jan. 15, 2021, which is a 371 national stage entry of International Application No. PCT/US2019/042494 filed Jul. 18, 2019, which claims priority to and the benefit of U.S. Provisional Application No. 62/700,118 filed Jul. 18, 2018, the contents of each of which are hereby incorporated herein by reference in their entirety.

### SEQUENCE LISTING

The instant application contains a Sequence Listing which has been submitted electronically in XML format, and is hereby incorporated by reference in its entirety. Said XML copy, created on May 9, 2025, is named 2003320_0187_SL.xml and is 49,082 bytes in size.

### BACKGROUND

Acyl amino acids are commercially important compounds. Many have advantageous characteristics and are sold as surfactants, antibiotics, anti-insect agents and as a variety of other important agents. Traditionally, acyl amino acids have been manufactured chemically. Such chemical manufacturing methods are hampered by a variety of shortcomings including the ease of obtaining and storing the starting materials, the necessity of using harsh and sometimes dangerous chemical reagents in the manufacturing process, the difficulty and efficiency of the synthesis itself, and/or the fiscal and environmental cost of disposing chemical by-products, etc.

### SUMMARY

The present inventors have previously described engineered polypeptides or engineered cells (see, for example, WO2008/131002 and WO2014/144649) that can be useful, among other things, for generation for acyl amino acids. Attributes of these engineered polypeptides or engineered cells addressed one or more shortcomings associated with chemical manufacturing of acyl amino acids as discussed above.

The present disclosure provides certain further insights and developments including, among other things, that acyl amino acids produced in microbial cells such as Bacillus cells, can be modified, for example, by hydroxylation and/or methylation, and that generation of such modified acyl amino acids can be controlled by use of strain engineering. In particular, for example, the present disclosure demonstrates that inactivation of a fatty acid hydroxylase (e.g., by inactivating cypB gene) in acyl amino acid-producing cells can reduce or eliminate hydroxylation of a fatty acid portion of an acyl amino acid at one or more ω-n positions, where n≥1 (e.g., ω-1, ω-2, and/or ω-3 positions). Such engineered constructs can be useful, for example, in certain circumstances where fatty acid hydroxylation results in production of such hydroxylated products as by-products, the presence of which in turn lowers the yield of desired surfactants and/or fatty acids that do not comprise additional hydroxyl groups (except that in some embodiments, such desirable surfactants and/or fatty acid may have a β-hydroxyl group). The present disclosure also provides, among other things, insights that in certain circumstances where hydroxylation of a fatty acid portion of an acyl amino acid at one or more ω-n positions, where n≥1 (e.g., ω-1, ω-2, and/or ω-3 positions) is desirable, such products can be produced by engineering cells to activate or over-express a gene encoding a fatty acid hydroxylase. Thus, the present disclosure teaches engineering acyl amino acid-producing cells to comprise a modification that modulates hydroxylation and/or alkylation (e.g., methylation) of a fatty acid portion of an acyl amino acid. Such technologies can be useful in increasing yield of surfactants and/or fatty acids of interest (e.g., with or without ω-n hydroxyl groups, where n≥1).

In some aspects, provided herein are engineered cells (e.g., engineered microbial cells) that are capable of producing acyl amino acids and modulating hydroxylation and/or methylation of a fatty acid portion of such acyl amino acids. In some embodiments, such an engineered cell (e.g., microbial cell) is an acyl amino acid-producing cell, which comprises a modification (e.g., a genetic modification) that modulates hydroxylation and/or methylation of a fatty acid portion of an acyl amino acid. In some embodiments, such an engineered microbial cell expresses at least one peptide synthetase, which produces or synthesizes an acyl amino acid.

In some embodiments involving a modification that modulates hydroxylation and/or methylation of a fatty acid potion of an acyl amino acid, such a modification may result in an increase in the number of hydroxyl and/or alkyl (e.g., methyl) groups of a fatty acid portion of an acyl amino acid, as compared to that when the modification is absent. In alternative embodiments, such a modification may result in a reduction in the number of hydroxyl and/or alkyl (e.g., methyl) groups of a fatty acid portion of an acyl amino acid, as compared to that when the modification is absent.

In some embodiments, such a modification that modulates hydroxylation and/or methylation of a fatty acid portion of an acyl amino acid is or comprises a modification in a gene that encodes a fatty acid modifying enzyme. In some embodiments, such a modification that modulates hydroxylation and/or methylation of a fatty acid portion of an acyl amino acid is or comprises a modification in one or more regulatory elements operably linked to a gene that encodes a fatty acid modifying enzyme. Depending on desirable types of acyl amino acids to be generated (e.g., with or without ω-n hydroxyl groups, where n≥1), in some embodiments, such a modification may be engineered to increase expression and/or activity of a gene that encodes a fatty acid modifying enzyme, while, in some embodiments, such a modification may be engineered to decrease expression of a gene that encodes a fatty acid modifying enzyme.

In some embodiments, a fatty acid modifying enzyme can be or comprise a fatty acid hydroxylase and/or a fatty acid methylase. In some embodiments, a fatty acid modifying enzyme is or comprises a fatty acid hydroxylase. In some such embodiments, a fatty acid hydroxylase may hydroxylate a linear or unbranched fatty acid moiety, while in some such embodiments, a fatty acid hydroxylase may hydroxylate a branched fatty acid moiety. In some embodiments, a fatty acid hydroxylase may be selected to hydroxylate a specific fatty acid moiety, including, e.g., but not limited to caproic acid, caprylic acid, lauric acid, and myristic acid. Examples of a fatty acid hydroxylase that may be involved in generation of acyl amino acids can be encoded by a gene selected from the group consisting of bioI, cyp107h, cyp107J1, cyp134A1, cyp109B1, cyp152A1, cyp102A2, cyp102A3, cyp107K1, and combinations thereof.

Various fatty acid modifying enzymes (e.g., fatty acid hydroxylases and/or fatty acid methylases) may be involved in generation of different kinds of acyl amino acids, for example, depending on the types of the fatty acid and/or amino acid moieties of acyl amino acids to be generated. One of ordinary skill in the art reading the present disclosure will appreciate that a proper fatty acid modifying enzyme may be selected for modification to modulate hydroxylation and/or methylation of a particular fatty acid portion of an acyl amino acid accordingly.

In some embodiments involving a peptide synthetase as described and/or utilized herein, such a peptide synthetase may be endogenous to a host cell. In some embodiments, a peptide synthetase may be heterologous to a host cell. In some embodiments, a peptide synthetase may be an engineered peptide synthetase. In some embodiments, a cell (e.g., a microbial cell) can be engineered to express a peptide synthetase. For example, in some embodiments, a cell (e.g., a microbial cell) can be engineered to contain a polynucleotide encoding a peptide synthetase.

In some embodiments involving cells (e.g., engineered cells) as described and/or utilized herein, such cells may be Bacillus cells, e.g., Bacillus subtilis cells.

Engineered cells described and/or utilized herein are useful for making an acyl amino acid composition. Accordingly, some aspects of the present disclosure provide methods of making an acyl amino acid composition using such engineered cells. In some embodiments, such a method comprises a step of (a) culturing an engineered cell (e.g., ones as described and/or utilized herein) under conditions and for a time sufficient for an acyl amino acid composition to be made.

In some embodiments involving culturing, such culturing can comprise incubating an engineered cell (e.g., ones as described and/or utilized herein) in a culture medium that comprises one or more of a carbon source, a fatty acid, and an amino acid. In some embodiments, such a culture medium may comprise a carbon source, a fatty acid, and an amino acid. In some embodiments involving making an acyl glycinate composition, an amino acid present in a culture medium is or comprises glycine. In some embodiments involving making an acyl glutamate composition, an amino acid present in a culture medium is or comprises glutamic acid. In some embodiments involving making an acyl sarcosinate composition, an amino acid present in a culture medium is or comprises sarcosine. One of ordinary skill in the art reading the present disclosure will appreciate that other amino acids can be present in a culture medium for use in making other acyl amino acids.

In some embodiments involving engineered cells comprising a modification (e.g., a genetic modification) that reduces hydroxylation of a fatty acid portion of an acyl amino acid, less than 10%, less than 7.5%, less than 5%, less than 2.5%, less than 1%, less than 0.5%, less than 0.2%, or less than 0.1% of the acyl amino acid in an acyl amino acid composition is hydroxylated at one or more ω-n positons (e.g., ω-1, ω-2, and/or ω-3 positions) of the fatty acid portion of the acyl amino acid, as compared to an acyl amino composition made using cells without such a modification. In some such embodiments, an acyl amino acid composition is substantially free of acyl amino acids that are hydroxylated at one or more ω-n positions (e.g., ω-1, ω-2, and/or ω-3 positon) of the fatty acid portion of the acyl amino acid.

In some embodiments involving engineered cells comprising a modification that reduces methylation of a fatty acid portion of an acyl amino acid, less than 10%, less than 7.5%, less than 5%, less than 2.5%, less than 1%, less than 0.5%, less than 0.2%, or less than 0.1% of the acyl amino acid in an acyl amino acid composition is methylated at one or more ω-n positons (e.g., ω-1, ω-2, and/or ω-3 positions) of the fatty acid portion of the acyl amino acid, as compared to an acyl amino composition made using cells without such a modification. In some such embodiments, an acyl amino acid composition is substantially free of acyl amino acids that are methylated at one or more ω-n positions (e.g., ω-1, ω-2, and/or ω-3 positon) of the fatty acid portion of the acyl amino acid.

In some embodiments involving engineered cells comprising a modification that increases hydroxylation of a fatty acid portion of an acyl amino acid, greater than 70% or more, including, e.g., greater than 90%, greater than 95%, greater than 98%, or greater than 99% of the acyl amino acid in an acyl amino acid composition is hydroxylated at one or more ω-n positions (e.g., ω-1, ω-2, and/or ω-3 positon) of the fatty acid portion of the acyl amino acid, as compared to an acyl amino composition made using cells without such a modification. In some such embodiments, an acyl amino acid composition is substantially entirely of acyl amino acids that are hydroxylated at one or more ω-n positions (e.g., ω-1, ω-2, and/or ω-3 positon) of the fatty acid portion of the acyl amino acid.

In some embodiments involving engineered cells comprising a modification that increases methylation of a fatty acid portion of an acyl amino acid, greater than 70% or more, including, e.g., greater than 90%, greater than 95%, greater than 98%, greater than 99%, or more, of the acyl amino acid in an acyl amino acid composition is methylated at one or more ω-n positions (e.g., ω-1, ω-2, and/or ω-3 positon) of the fatty acid portion of the acyl amino acid, as compared to an acyl amino composition made using cells without such a modification. In some such embodiments, an acyl amino acid composition is substantially entirely of acyl amino acids that are methylated at one or more ω-n positions (e.g., ω-1, ω-2, and/or ω-3 positon) of the fatty acid portion of the acyl amino acid.

Technologies described and/or utilized herein are useful for generation of various acyl amino acids. In some embodiments, technologies provided herein can be useful for generation of acyl glycinate. In some embodiments, technologies provided herein can be useful for generation of acyl glutamate. In some embodiments, technologies provided herein can be useful for generation of acyl sarconsinate.

### BRIEF DESCRIPTION OF THE DRAWING

FIG. 1 presents a depiction of initial steps of fatty acid synthesis with an acetyl initiation moiety; the depicted steps achieve full saturation of the chain.

FIG. 2 depicts exemplary acyl amino acids produced by engineered enzymes and/or microbial cells described and/or utilized herein. The present inventors have previously developed engineered enzymes and/or microbial cells that catalyze production of an acyl amino acid (for example, β-hydroxy myristoyl glutamate), which engineered enzymes and/or microbial cells comprise at least one domain that is specific for a particular fatty acid and at least one domain that is specific for a particular amino acid. Such engineered enzymes are modular. In FIG. 2, the left-most rectangle represents a block of DNA that encodes an enzyme module, which catalyzes addition of a particular fatty acid (e.g., myristic acid (14 carbon)) onto a specific amino acid. When the “amino acid specifying” block of code is one particular sequence (e.g., glutamic acid), the fatty acids are linked to the amino acid glutamate. Acyl glutamate surfactant produced by such an engineered enzyme is referred to as FA-Glu (Fatty Acid linked to Glutamate, or AminoSurf-E). When the amino acid specifying block of code is a different sequence (e.g., glycine), the fatty acids are linked to the amino acid glycine. Acyl glycinate surfactant produced by such an engineered enzyme is referred to as FA-Gly (Fatty Acid linked to Glycine, or AminoSurf-G).

FIG. 3 depicts LC-MS analysis of an exemplary acyl acid composition produced by fermentation supplemented with glycine and caprylic acid.

### CERTAIN DEFINITIONS

Acyl amino acid: The term “acyl amino acid” as used herein refers to an amino acid moiety that is covalently linked to a fatty acid moiety. In some embodiments, the amino acid and fatty acid moieties are covalently linked via an amide bond formed between a carboxylic acid group of a fatty acid and an amino group of an amino acid. In some embodiments, a fatty acid moiety or entity utilized or included in an acyl amino acid includes a β-hydroxyl group; in some embodiments, a fatty acid moiety or entity utilized or included in an acyl amino acid does not include a β-hydroxyl group. In some embodiments, a fatty acid moiety utilized or included in an acyl amino acid includes a β-amino group; in some embodiments, a fatty acid moiety or entity utilized or included in an acyl amino acid does not include a β-amino group. In some embodiments, a fatty acid moiety utilized or included in an acyl amino acid is unmodified at the β-position. In some embodiments, a fatty acid moiety utilized or included in an acyl amino acid is modified (e.g., by hydroxylation) at the β-position.

Acyl amino acid-producing cell: The term “acyl amino acid-producing cell” refers to a biological cell that produces or synthesizes an acyl amino acid (e.g., as described herein). In some embodiments, an acyl amino acid-producing cell expresses an enzyme that is capable of covalently associates a fatty acid with an amino acid. In some embodiments, such an enzyme may be an endogenous enzyme. In some embodiments, such an enzyme may be a heterologous enzyme. In some embodiments, such an enzyme may be an engineered enzyme. In some embodiments, such an enzyme may be or comprise a peptide synthetase (e.g., ones as described herein). In some embodiments, an acyl amino acid-producing cell may comprise a modification (e.g., a genetic modification) such that expression and/or activity of a fatty acid modifying enzyme is modulated (e.g., increased or reduced) in the acyl amino acid-producing cell, as compared to that of a fatty acid modifying enzyme without such a modification.

Amino acid: As used herein, the term “amino acid,” in its broadest sense, refers to any compound and/or substance that can be utilized in peptide synthesis (e.g., ribosomal or non-ribosomal synthesis). In some embodiments, an amino acid is any compound and/or substance that can be incorporated into a polypeptide chain, e.g., through formation of one or more peptide bonds. In some embodiments, an amino acid is any compound and/or substance that is a substrate for a peptide synthetase; in some such embodiments, an amino acid is any compound and/or substance onto which a peptide synthetase can link an acyl entity, for example through formation of an amide bond. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, and/or substitution as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” is used to refer to a free amino acid; in some embodiments it is used to refer to an amino acid residue of a polypeptide or an acyl amino acid. In some embodiments, a “naturally occurring” amino acid is one of the standard group of twenty amino acids that are the building blocks of polypeptides of most organisms, including alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments a “naturally occurring” amino acid may be one of those amino acids that are used less frequently and are typically not included in this standard group of twenty but are nevertheless still used by one or more organisms and incorporated into certain polypeptides. For example, the codons UAG and UGA normally encode stop codons in most organisms. However, in some organisms the codons UAG and UGA encode the amino acids selenocysteine and pyrrolysine. Thus, in certain embodiments, selenocysteine and pyrrolysine are naturally occurring amino acids.

Associated with: Two events or entities are “associated” with one another, as that term is used herein, if the presence, level and/or form of one is correlated with that of the other. For example, a particular entity (e.g., gene) is considered to be associated with a biological characteristic and/or function, if its presence, level, activity, and/or form correlates with the presence, absence, and/or level of the biological characteristic and/or function. As another example, a particular entity (e.g., polypeptide) is considered to be associated with a particular disease, disorder, or condition, if its presence, level and/or form correlates with incidence of and/or susceptibility of the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Beta-hydroxy fatty acid: The term “beta-hydroxy fatty acid” as used herein refers to a fatty acid moiety (e.g., as described herein) comprising a hydroxy group at the beta position of the fatty acid moiety. As is understood by those skilled in the art, the beta position corresponds to the third carbon of the fatty acid chain, the first carbon being the carbon of the carboxylate group. Thus, when used in reference to an acyl amino acid (e.g., ones as described herein), where an amide moiety is formed between the carboxylate moiety of a fatty acid moiety and the nitrogen of an amino acid moiety, the beta position corresponds to the third carbon of the fatty acid moiety, with the first carbon being the carbon of the amide moiety. A beta-hydroxy fatty acid for use in accordance with the present disclosure may contain a linear or straight carbon chain. Additionally or alternatively, a beta-hydroxy fatty acid for use in accordance with the present disclosure may be a branched-chain fatty acid. In some such embodiments, a beta-hydroxy fatty acid may be terminally branched (i.e., having a branched carbon chain at one end of the fatty acid). In some such embodiments, a beta-hydroxy fatty acid may be a branched fatty acid of an iso type. In some embodiments, a beta-hydroxy fatty acid may be a branched fatty acid of an anteiso type. In some embodiments, a beta-hydroxy fatty acid for use in accordance with the present disclosure may be a mono-unsaturated or poly-unsaturated fatty acid. Alternatively, a beta-hydroxy fatty acid for use in accordance with the present disclosure may be a saturated fatty acid. A beta-hydroxy fatty acid for use in accordance with the present disclosure may contain any number of carbon atoms in the fatty acid chain. As non-limiting examples, a beta-hydroxy fatty acid may contain 3, 4, 5, 6, 7, 8, 9, 10, 11, 12, 3, 14, 15, 15, 16, 17, 18, 19, 20, 21, 22, 23, 24, 25, 26, 27, 28, 29, 30 or more carbon atoms. In some embodiments, a beta-hydroxy fatty acid that may be used in accordance with the present disclosure contains 13 to 15 carbons in the fatty acid chain. In some embodiments, a beta-hydroxy fatty acid is or comprises a saturated or unsaturated long-chain fatty acid with a 14-carbon backbone. In some embodiments, a beta-hydroxy fatty acid is or comprises myristic acid. Those of ordinary skill in the art will be aware of various beta-hydroxy fatty acids that can be used in accordance with the present disclosure.

Beta-hydroxyfatty acid linkage domain: The term “beta-hydroxy fatty acid linkage domain” as used herein refers to a domain (e.g., a polypeptide domain) that covalently links a beta-hydroxy fatty acid to an amino acid to form an acyl amino acid. A variety of beta-hydroxy fatty acid linkage domains are known to those skilled in the art. As will be understood by those skilled in the art, various beta-hydroxy fatty acid linkage domains typically exhibit specificity for one or more beta-hydroxy fatty acids. As one non-limiting example, a beta-hydroxy fatty acid linkage domain from surfactin synthetase is specific for beta-hydroxy myristic acid. Thus, in some embodiments, a beta-hydroxy fatty acid linkage domain from surfactin synthetase can be used in accordance with the present disclosure to construct an engineered polypeptide useful in the generation of an acyl amino acid in which beta-hydroxy myristic acid constitutes the fatty acid moiety or portion of the acyl amino acid. Different beta-hydroxy fatty acid linkage domains that exhibit specificity for other beta-hydroxy fatty acids (e.g., naturally or non-naturally occurring beta-hydroxy fatty acids) may be used in accordance with the present disclosure to generate any acyl amino acid of the practitioner's choosing.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

Comparable: The term “comparable”, as used herein, refers to two or more agents, entities, situations, sets of conditions, etc. that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

Corresponding to: As used herein, the term “corresponding to” is often used to designate the position/identity of a residue in a polymer, such as an amino acid residue in a polypeptide or a nucleotide residue in a nucleic acid. Those of ordinary skill will appreciate that, for purposes of simplicity, residues in such a polymer are often designated using a canonical numbering system based on a reference related polymer, so that a residue in a first polymer “corresponding to” a residue at position 190 in the reference polymer, for example, need not actually be the 190^{th} residue in the first polymer but rather corresponds to the residue found at the 190^{th} position in the reference polymer; those of ordinary skill in the art readily appreciate how to identify “corresponding” amino acids, including through use of one or more commercially-available algorithms specifically designed for polymer sequence comparisons.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, α-helix character, β-sheet character, coiled-coil character, random coil character, etc.), and/or by a particular functional feature (e.g., binding activity, enzymatic activity, folding activity, signaling activity, activity of linking two entities together etc.). In some embodiments, a domain can be an engineered domain. For example, in some embodiments, an engineered domain may refer to an engineered polypeptide moiety that correspond and/or show significant homology and/or identity to a naturally occurring polypeptide moiety, or to a reference polypeptide moiety. In some such embodiments, an engineered domain may share a characteristic structure (e.g., primary structure such as the amino acid sequence of a domain, and/or secondary, tertiary, quaternary, etc. structures); alternatively or additionally, such an engineered domain may exhibit one or more distinct functions that it shares with a reference polypeptide moiety. As will be understood by those skilled in the art, in many cases polypeptides are modular and may comprise one or more polypeptide domains; in some such embodiments, each domain can exhibit one or more distinct functions or characteristics of a polypeptide; or in some such embodiments, a plurality of domains may contribute to the overall function of a polypeptide. In some embodiments, the structure and/or function of many such domains are known to those skilled in the art.

Endogenous: As used herein, the term “endogenous” refers to a substance or process that is present or occurs naturally, e.g., in a non-recombinant host cell.

Engineered: Those of ordinary skill in the art, reading the present disclosure, will appreciate that the term “engineered”, as used herein, refers to an aspect of having been manipulated and altered by the hand of man. For example, an engineered cell (e.g., an engineered microbial cell) refers to a cell that has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation. In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. In reference to a polypeptide, an “engineered polypeptide” refers to a polypeptide that has been designed and/or produced by the hand of man. In some embodiments, an engineered polypeptide has an amino acid sequence that includes one or more sequence elements that do(es) not occur in nature. In some embodiments, an engineered polypeptide has an amino acid sequence that includes one or more sequence elements that does occur in nature, but that is present in the engineered polypeptide in a different sequence context (e.g., separated from at least one sequence to which it is linked in nature and/or linked with at least one sequence element to which it is not linked in nature) from that in which it occurs in nature. In some embodiments, an engineered polypeptide is one in which naturally-occurring sequence element(s) is/are separated from at least one sequence with which they/it is associated (e.g., linked) in nature and/or is otherwise manipulated to comprise a polypeptide that does not exist in nature. In various embodiments, an engineered polypeptide comprises two or more covalently linked polypeptide domains. Typically such domains may be linked via peptide bonds or other covalent linkages known to those skilled in the art. One or more covalently linked polypeptide domains of engineered polypeptides may be naturally occurring. Thus, in certain embodiments, engineered polypeptides described herein may comprise two or more covalently linked domains, at least one of which is naturally occurring. In certain embodiments, two or more naturally occurring polypeptide domains are covalently linked to generate an engineered polypeptide. For example, naturally occurring polypeptide domains from two or more different polypeptides may be covalently linked to generate an engineered polypeptide. In certain embodiments, naturally occurring polypeptide domains of an engineered polypeptide are covalently linked in nature, but are covalently linked in the engineered polypeptide in a way that is different from the way the domains are linked nature. For example, two polypeptide domains that naturally occur in the same polypeptide but which are separated by one or more intervening amino acid residues may be directly covalently linked (e.g., by removing the intervening amino acid residues) to generate an engineered polypeptide. Additionally or alternatively, two polypeptide domains that naturally occur in the same polypeptide which are directly covalently linked together (e.g., not separated by one or more intervening amino acid residues) may be indirectly covalently linked (e.g., by inserting one or more intervening amino acid residues) to generate an engineered polypeptide. In certain embodiments, one or more covalently linked polypeptide domains of an engineered polypeptide may not exist naturally. For example, such polypeptide domains may be engineered themselves.

Enriched: As used herein, the term “enriched” refers to an increase in the proportion of one or more components of a composition. For examples, in some embodiments, an acyl amino acid composition produced by technologies described herein may comprise a plurality of distinct acyl amino acids but is enriched in one or a few acyl amino acid (e.g., of a certain carbon length of a fatty acid portion), as compared to a reference product (e.g., an acyl amino acid composition that is not produced by technologies described herein). In some such embodiments, an acyl amino acid composition produced by technologies described herein contains a higher proportion of an acyl amino acid having a particular carbon length of a fatty acid portion (e.g., C14 for a fatty acid portion) than that of a reference product (e.g., an acyl amino acid composition that is not produced by technologies described herein), for example, by at least 10%, including, e.g., at least 20%, at least 30%, at least 40%, at least 50%, at least 60%, at least 70%, at least 80%, at least 90%, or more.

Expression: As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fatty acid: As used herein, the term “fatty acid” refers to a moiety having a carboxylic acid or carboxyl moiety with an aliphatic tail, e.g., in some embodiments, from 3 to 30 carbon atoms long. As will be understood by one of skill in the art, the carbon next to the carboxylic acid moiety is designated as a; the next one is 3, and so forth, while the carbon at the tail of a fatty acid (i.e., the carbon that is most distant from the carboxylic acid moiety of a fatty acid) is designated as ω. Typically, carbon atoms that are close to the ω carbon are designated in relation to the ω carbon. For example, the third carbon starting from the ω carbon is designated as ω-3 carbon. Accordingly, a carbon atom at a (ω-n) position of a fatty acid refers to the n^{th} carbon atom starting from the ω carbon, wherein n is 2 or higher (up to the length of the carbon chain), e.g., n is 2, 3, 4, 5, 6, etc. Fatty acids can be saturated, mono-unsaturated or poly-unsaturated. Fatty acids can be straight chain or branched (e.g., iso or anteiso). In some embodiments, a fatty acid may include one or more hydroxyl group. In some such embodiments, a fatty acid may be or comprises a beta-hydroxy fatty acid as described herein. Examples of fatty acids useful in the disclosure, include, but are not limited to, butyric acid (C4), caproic acid (C6), caprylic acid (C8), capric acid (C10), lauric acid (C12), myristic acid (C14), palmitic acid (C16), palmitoleic acid (C16), stearic acid (C18), isostearic acid (C18), oleic acid (C18), vaccenic acid (C18), linoleic acid (C18), alpha-linolenic acid (C18), gamma-linolenic acid (C18), arachidic acid (C20), gadoleic acid (C20), arachidonic acid (C20), eicosapentaenoic acid (C20), behenic acid (C22), crude acid (C22), docosahexaenoic acid (C22), lignoceric acid (C24) and hexacosanoic acid (C26). One of skill in the art will appreciate that other fatty acids may be useful for technologies described herein. In some aspects, a fatty acid may refer to a fatty acid portion of an acyl amino acid.

Fatty acid hydroxylase: As used herein, the term “fatty acid hydroxylase” generally refers to an enzyme or a functional domain thereof that adds a hydroxyl group to at least one carbon within the backbone of a fatty acid moiety. In some embodiments, a fatty acid hydroxylase may have specificity for a fatty acid of a particular structure (e.g., a branched fatty acid vs. a linear or straight fatty acid). In some embodiments, a fatty acid hydroxylase may have specificity for a fatty acid of a specific carbon length. For example, in some embodiments, a fatty acid hydroxylase may have specificity for a fatty acid having a carbon length of at least 3 or more, including, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, or more. In some embodiments, a fatty acid hydroxylase may have specificity for adding a hydroxyl group to the carbon atom at one or more particular positions within the backbone of a fatty acid moiety. For example, in some embodiments, a fatty acid hydroxylase may have specificity for adding a hydroxyl group to the carbon atom at ω-1, ω-2, and/or ω-3 position of a fatty acid moiety.

Fatty acid linkage domain: The term “fatty acid linkage domain” as used herein refers to a domain (e.g., a polypeptide domain) that covalently links a fatty acid to an amino acid to form an acyl amino acid. In some embodiments, a fatty acid linkage domain is or comprises a condensation domain; in some embodiments such a fatty acid linkage domain is part of a single polypeptide or a polypeptide complex with at least or only an adenylation domain, a thiolation domain, or both. A variety of fatty acid linkage domains are known in the art, such as for example, fatty acid linkage domains that are present in various peptide synthetase complexes that produce lipopeptides. In certain embodiments, a fatty acid linkage domain is or comprises a beta-hydroxy fatty acid linkage domain as described herein, e.g., linking a beta-hydroxy fatty acid to an amino acid. In some embodiments, a fatty acid linkage domain links a beta-amino fatty acid to an amino acid. In some embodiments, a fatty acid linkage domain links a fatty acid that is not modified at the beta position to an amino acid. In some embodiments, a fatty acid linkage domain links a fatty acid that does not have a hydroxyl group at the beta position to an amino acid. In some embodiments, a fatty acid linkage domain catalyzes condensation of a fatty acid and an amino acid so that an amide bond is formed, for example between a carboxylic acid moiety on a fatty acid and an amino moiety on an amino acid. In some embodiments, a fatty acid linkage domain is or comprises a domain that is at least 70% or more, including, e.g., at least 75%, at least 80%, at least 85%, at least 90%, at least 95% or higher (and up to 100%), identical at the amino acid level to that found in Bacillus subtilis's surfactin synthetase SrfA protein.

Fatty acid methylase: As used herein, the term “fatty acid methylase” generally refers to an enzyme or a functional domain thereof that adds a methyl group to at least one carbon within the backbone of a fatty acid moiety. In some embodiments, a fatty acid methylase may have specificity for a fatty acid of a particular structure (e.g., a branched fatty acid vs. a linear or straight fatty acid). In some embodiments, a fatty acid methylase may have specificity for a fatty acid of a specific carbon length. For example, in some embodiments, a fatty acid methylase may have specificity for a fatty acid having a carbon length of at least 3 or more, including, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, or more. In some embodiments, a fatty acid methylase may have specificity for adding a methyl group to the carbon atom at one or more particular positions within the backbone of a fatty acid moiety. For example, in some embodiments, a fatty acid methylase may have specificity for adding a methyl group to the carbon atom at ω-1, ω-2, and/or ω-3 position of a fatty acid moiety.

Fatty acid modifying enzyme: As used herein, the term “fatty acid modifying enzyme” refers to an enzyme or a functional domain thereof that is capable of modifying one or more structural features of a fatty acid moiety (e.g., a fatty acid portion, such as a fatty acid backbone portion, of an acyl amino acid). Examples of such structural modifications include, but are not limited to hydroxylation and/or alkylation (e.g., methylation) of one or more carbon atoms within the backbone chain of a fatty acid moiety. In some embodiments, a fatty acid modifying enzyme may link a hydroxyl group to one or more carbon atoms of a fatty acid backbone chain. In some such embodiments, a fatty acid modifying enzyme may link a hydroxyl group to one or carbon atoms at the ω-n position (e.g., ω-1 position, ω-2 position, ω-3 position, etc.) of a fatty acid moiety (e.g., a fatty acid portion of an acyl amino acid). In some embodiments, a fatty acid modifying enzyme may link an alkyl group (e.g., C1-C5 such as a methyl group) to one or more carbon atoms of a fatty acid backbone chain. In some such embodiments, a fatty acid modifying enzyme may link an alkyl group (e.g., C1-C5 such as a methyl group) to one or carbon atoms at the ω-n position (e.g., ω-1 position, ω-2 position, ω-3 position, etc.) of a fatty acid moiety (e.g., a fatty acid portion of an acyl amino acid). In some embodiments, a fatty acid modifying enzyme may have specificity for a fatty acid of a particular structure (e.g., a branched fatty acid vs. a linear or straight fatty acid). In some embodiments, a fatty acid modifying enzyme may have specificity for a fatty acid of a specific carbon length. For example, in some embodiments, a fatty acid modifying enzyme may have specificity for a fatty acid having a carbon length of at least 3 or more, including, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, or more. In some embodiments, a fatty acid modifying enzyme may have specificity for introduction of a modification (e.g., hydroxylation or methylation) to the carbon atom at one or more particular positions within the backbone of a fatty acid moiety. For example, in some embodiments, a fatty acid modifying enzyme may have specificity for introduction of a modification (e.g., hydroxylation or methylation) to the carbon atom at ω-1, ω-2, and/or ω-3 position of a fatty acid moiety.

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Genetic modification: As used herein, the term “genetic modification” refers to stable or transient alteration of expression and/or activity of a gene by introduction of exogenous DNA into a host cell. Exogenous DNA may be synthetic, or naturally derived, and may contain one or more genes, portions of one or more genes, or other useful DNA sequences. Exogenous DNA may be introduced to a cell by methods known in the art; one of ordinary skill in the art will appreciate that in some embodiments, viral vectors (e.g., retrovirus, modified herpes viral, herpes-viral, adenovirus, adeno-associated virus, and the like) may be used to introduce exogenous DNA into a host cell, or in some embodiments, direct DNA transfection (e.g., lipofection, calcium phosphate transfection, DEAE-dextran, electroporation, and the like) may be used. Other gene editing methods that are known in the art can also be used to carry out genetic modification. In some embodiments, a genetic modification can comprise an insertion of one or more nucleotides, a deletion of one or more nucleotides, a substitution of one or more nucleotides, a knockout, a knockin, a point mutation, a replacement of an endogenous nucleic acid sequence with a homologous, heterologous, or orthologous nucleic acid sequence, or a combination thereof.

Heterologous: As used herein, the term “heterologous” as used herein refers to a entity (e.g., nucleic acid or polypeptide) wherein at least one of the following is true: (a) the entity (e.g., nucleic acid or polypeptide) is foreign (“exogenous”) to (that is, not naturally found in) a given host cell; (b) the entity (e.g., nucleic acid or polypeptide) comprises a nucleotide sequence that is naturally found in (that is, is “endogenous to”) a given host cell, but the nucleotide sequence is produced in an unnatural (for example, greater than expected or greater than naturally found) amount in the cell; (c) the entity (e.g., nucleic acid or polypeptide) comprises a sequence that differs in sequence from an endogenous sequence, but the sequence encodes the same protein (having the same or substantially the same amino acid sequence) and is produced in an unnatural (for example, greater than expected or greater than naturally found) amount in the cell; or (d) the entity (e.g., nucleic acid or polypeptide) comprises two or more sequences that are not found in the same relationship to each other in nature (for example, the sequence is recombinant).

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino | | |
|---|---|---|
| Acids | 3-Letter | 1-Letter |
| Asparagine or | Asx | B |
| aspartic acid | | |
| Glutamine or | Glx | Z |
| glutamic acid | | |
| Leucine or Isoleucine | Xle | J |
| Unspecified or | Xaa | X |
| unknown amino acid | | |

As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna.CMP matrix.

Host cell: As used herein, refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, a host cell is a microbial cell such as, e.g., a bacterial cell.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent identity between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. Representative algorithms and computer programs useful in determining the percent identity between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined for example using the GAP program in the GCG software package using an NWSgapdna.CMP matrix.

Increased or reduced: As used herein, these terms, or grammatically comparable comparative terms, indicate values that are relative to a comparable reference measurement. For example, in some embodiments, an assessed value achieved with an agent of interest may be “increased” relative to that obtained with a comparable reference agent. Alternatively or additionally, in some embodiments, an assessed value achieved in a subject or system of interest may be “increased” relative to that obtained in the same subject or system under different conditions (e.g., in the presence or absence of an agent of interest), or in a different, comparable subject or system (e.g., in a comparable subject or system that differs from the subject or system of interest in presence or absence of an agent of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Moiety: Those skilled in the art will appreciate that a “moiety” is a defined chemical group or entity with a particular structure and/or or activity, as described herein.

Modulate: As used herein, the term “modulate” means either to cause a change in level and/or nature of an activity of interest. In some embodiments, modulation may refer to an increase in the level of an activity of interest, as compared to a reference level. In some embodiments, modulate may refer to a reduction in the level of an activity of interest, as compared to a reference level.

Isolated: As used herein, the term “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced. In some embodiments, isolation involves or requires disruption of covalent bonds (e.g., to isolate a polypeptide domain from a longer polypeptide and/or to isolate a nucleotide sequence element from a longer oligonucleotide or nucleic acid).

Naturally occurring: The term “naturally occurring”, as used herein, refers to an agent or entity that is known to exist in nature.

Nucleic acid. As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Operably linked: as used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Peptide synthetase: The term “peptide synthetase”, as used interchangeably with the term “peptide synthetase complex”, refers to an enzyme that catalyzes the non-ribosomal production of peptides. As will be appreciated by those of ordinary skill in the art, peptide synthetase complexes are modular, and comprise individual peptide synthetase modules that perform different steps in the synthesis of the ultimate peptide; typically, each module performs one step (e.g., adds a single amino acid). A peptide synthetase complex may comprise a single enzymatic subunit (e.g., a single polypeptide), or may comprise two or more enzymatic subunits (e.g., two or more polypeptides). In some embodiments, a peptide synthetase complex may comprise at least one peptide synthetase domain, and may further comprise one or more additional domains such as for example, a fatty acid linkage domain, a thioesterase domain, a reductase domain, etc. In some embodiments, a peptide synthetase domain of a peptide synthetase complex may comprise two or more enzymatic subunits, with two or more peptide synthetase domains present in a given enzymatic subunit. For example the surfactin peptide synthetase complex (also referred to herein simply as “surfactin synthetase complex”) comprises three distinct polypeptide enzymatic subunits: the first two subunits comprise three peptide synthetase domains, while the third subunit comprises a single peptide synthetase domain.

Peptide synthetase domain: The term “peptide synthetase domain” as used herein refers to a domain of a peptide synthetase. In some embodiments, a peptide synthetase domain minimally comprises three domains: an adenylation (A) domain (which is capable of selectively recognizing and activating a specific amino acid), a thiolation (T) domain (which is capable of tethering an activated amino acid to a cofactor via thioester linkage), and a condensation (C) domain (which is capable of linking one or more amino acids to successive units of a peptide synthetase through formation of amide bonds. A peptide synthetase domain typically recognizes and activates a single, specific amino acid, and in certain situations where the peptide synthetase domain is not the first domain in a peptide synthesis pathway, links the specific amino acid to the growing peptide chain. In some embodiments, a peptide synthetase domain may be or comprise a domain that is at least 70% or more, including, e.g., at least 75%, at least 80%, at least 85%, at least 90%, at least 95% or higher (and up to 100%), identical at the amino acid level to Bacillus subtilis's surfactin synthetase complex SrfA-A polypeptide subunit's first peptide synthetase domain.

Polypeptide: The term “polypeptide” as used herein refers to a polymer of at least three amino acid residues. In some embodiments, a “polypeptide” has a structure as achieved through synthesis by ribosomal machinery in naturally occurring organisms. In some embodiments a “polypeptide” has a structure as achieved through chemical synthesis (e.g., in vitro). In some embodiments, a “polypeptide” has a structure as achieved through joining of a series of amino acids joined together by non-ribosomal machinery, such as by way of non-limiting example, polypeptides synthesized by peptide synthetases. Such non-ribosomally produced polypeptides exhibit a greater diversity in covalent linkages than polypeptides synthesized by ribosomes (although those skilled in the art will understand that the amino acids of ribosomally-produced polypeptides may also be linked by covalent bonds that are not peptide bonds, such as the linkage of cystines via disulfide bonds). In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reductase Domain: The term “reductase domain” as used herein refers to a domain (e.g., polypeptide domain) that catalyzes release of an acyl amino acid produced by a peptide synthetase complex from the peptide synthetase complex. In certain embodiments, a reductase domain is covalently linked to a peptide synthetase domain and a fatty acid linkage domain such as a beta-hydroxy fatty acid linkage domain to generate an engineered polypeptide useful in the synthesis of an acyl amino acid. A variety of reductase domains are found in nature in nonribosomal peptide synthetase complexes from a variety of species. A non-limiting example of a reductase domain that may be used in accordance with the present disclosure includes the reductase domain from linear gramicidin (ATCC8185). However, a reductase domain that releases an acyl amino acid produced by a peptide synthetase complex from the peptide synthetase complex may be used in accordance with the present disclosure. In some embodiments, reductase domains are characterized by the presence of the consensus sequence: [LIVSPADNK]-x(9)-{P}-x(2)-Y-[PSTAGNCV]-[STAGNQCIVM]-[STAGC]-K-{PC}-[SAGFYR]-[LIVMSTAGD]-x-{K}-[LIVMFYW]-{D}-x-{YR}-[LIVMFYWGAPTHQ]-[GSACQRHM](SEQ ID NO: 21), where square brackets (“[ ]”) indicate amino acids that are typically present at that position, squiggly brackets (“{ }”) indicate amino acids that amino acids that are typically not present at that position, and “x” denotes any amino acid or a gap. X(9) for example denotes any amino acids or gaps for nine consecutive positions. Those skilled in the art will be aware of methods to determine whether a give polypeptide domain is a reductase domain. In some embodiments, a reductase domain is or comprises a domain that is at least 70% or more, including, e.g., at least 75%, at least 80%, at least 85%, at least 90%, at least 95%, or more (and up to 100%), identical at the amino acid level to that found in Bacillus brevis's linear gramicidin synthetase complex.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Regulatory element: As used herein, the term “regulatory element” refers to an entity (e.g., a polynucleotide entity) having gene regulatory activity, i.e. one that has the ability to affect the transcription or translation of an operably linked polynucleotide molecule (e.g., a gene encoding a polypeptide of interest). Regulatory elements including, e.g., but not limited to promoters, leaders, introns, and transcription termination regions, are molecules having gene regulatory activity which play an integral part in the overall expression of genes in biological cells. In some embodiments, by “regulatory element” it is intended to mean a series of nucleotides that determines if, when, and at what level a particular gene is expressed. The regulatory DNA sequences specifically interact with regulatory proteins or other proteins.

Specific: The term “specific” or “specificity” when used herein with reference to an entity having an activity, is understood by those skilled in the art to mean that the entity discriminates between potential target entities or states. For example, in some embodiments, a fatty acid linkage domain that is said to link a “specific” fatty acid to an amino acid if it links preferentially that target fatty acid to an amino acid in the presence of one or more competing alternative fatty acids. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of a domain for one or more other potential target entities (e.g., competitors). In some embodiments, specificity is evaluated relative to that of a reference specific domain. In some embodiments specificity is evaluated relative to that of a reference non-specific domain.

Small molecule: As used herein, the term “small molecule” means a low molecular weight organic compound that may serve as an enzyme substrate or regulator of biological processes. In general, a “small molecule” is a molecule that is less than about 5 kilodaltons (kD) in size. In some embodiments, provided nanoparticles further include one or more small molecules. In some embodiments, the small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, one or more small molecules are encapsulated within the nanoparticle. In some embodiments, small molecules are non-polymeric. In some embodiments, in accordance with the present disclosure, small molecules are not proteins, polypeptides, oligopeptides, peptides, polynucleotides, oligonucleotides, polysaccharides, glycoproteins, proteoglycans, etc. In some embodiments, a small molecule is a therapeutic. In some embodiments, a small molecule is an adjuvant. In some embodiments, a small molecule is a drug.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Surfactin: Surfactin is cyclic lipopeptide that is naturally produced by certain bacteria, including the Gram-positive endospore-forming bacteria Bacillus subtilis. Surfactin is an amphiphilic molecule (having both hydrophobic and hydrophilic properties) and is thus soluble in both organic solvents and water. Surfactin exhibits exceptional surfactant properties, making it a commercially valuable molecule. Due to its surfactant properties, surfactin also functions as an antibiotic. For example, surfactin is known to be effective as an anti-bacterial, anti-viral, anti-fungal, anti-mycoplasma and hemolytic compound. Surfactin is capable of penetrating the cell membranes of all types of bacteria, including both Gram-negative and Gram-positive bacteria, which differ in the composition of their membrane. Gram-positive bacteria have a thick peptidoglycan layer on the outside of their phospholipid bilayer. In contrast, Gram-negative bacteria have a thinner peptidoglycan layer on the outside of their phospholipid bilayer, and further contain an additional outer lipopolysaccharide membrane. Surfactin's surfactant activity permits it to create a permeable environment for the lipid bilayer and causes disruption that solubilizes the membrane of both types of bacteria. In order for surfactin to carry out minimal antibacterial effects, the minimum inhibitory concentration (MIC) is in the range of 12-50 μg/ml. In addition to its antibacterial properties, surfactin also exhibits antiviral properties, and its known to disrupt enveloped viruses such as HIV and HSV. Surfactin not only disrupts the lipid envelope of viruses, but also their capsids through ion channel formations. Surfactin isoforms containing fatty acid chains with 14 or 15 carbon atoms exhibited improved viral inactivation, thought to be due to improved disruption of the viral envelope. Surfactin consists of a seven amino acid peptide loop, and a hydrophobic fatty acid chain (beta-hydroxy myristic acid) that is thirteen to fifteen carbons long. The fatty acid chain allows permits surfactin to penetrate cellular membranes. The peptide loop comprises the amino acids L-asparagine, L-leucine, glycine, L-leucine, L-valine and two D-leucines. Glycine and asparagine residues at positions 1 and 6 respectively, constitute a minor polar domain. On the opposite side, valine residue at position 4 extends down facing the fatty acid chain, making up a major hydrophobic domain. Surfactin is synthesized by the surfactin synthetase complex, which comprises the three surfactin synthetase polypeptide subunits SrfA-A, SrfA-B, and SrfA-C. The surfactin synthetase polypeptide subunits SrfA-A and SrfA-B each comprise three peptide synthetase domains, each of which adds a single amino acid to the growing surfactin peptide, while the monomodular surfactin synthetase polypeptide subunit SrfA-C comprises a single peptide synthetase domain and adds the last amino acid residue to the heptapeptide. Additionally the SrfA-C subunit comprises a thioesterase domain, which catalyzes the release of the product via a nucleophilic attack of the beta-hydroxy of the fatty acid on the carbonyl of the C-terminal Leu of the peptide, cyclizing the molecule via formation of an ester. The spectrum of the beta-hydroxy fatty acids was elucidated as iso, anteiso C13, iso, normal C14 and iso, anteiso C15, and a recent study has indicated that surfactin retains an R configuration at C-beta (Nagai et al., Study on surfactin, a cyclic depsipeptide. 2. Synthesis of surfactin B2 produced by Bacillus natto KMD 2311. Chem Pharm Bull (Tokyo) 44: 5-10, 1996).

Surfactin is a lipopeptide synthesized by the surfactin synthetase complex. Surfactin comprises seven amino acids, which are initially joined by peptide bonds, as well as a beta-hydroxy fatty acid covalently linked to the first amino acid, glutamate. However, upon addition the final amino acid (leucine), the polypeptide is released and the thioesterase domain of the SRFC protein catalyzes the release of the product via a nucleophilic attack of the beta-hydroxy of the fatty acid on the carbonyl of the C-terminal Leu of the peptide, cyclizing the molecule via formation of an ester, resulting in the C-terminus carboxyl group of leucine attached via a lactone bond to the b-hydroxyl group of the fatty acid.

Thioesterase domain: The term “thioesterase domain” as used herein refers to a polypeptide domain that catalyzes release of an acyl amino acid produced by a peptide synthetase complex from the peptide synthetase complex. A variety of thioesterase domains are found in nature in nonribosomal peptide synthetase complexes from a variety of species. A non-limiting example of a thioesterase domain that may be useful in technologies provided herein includes the thioesterase domain from the Bacillus subtilis surfactin synthetase complex, present in Srf-C subunit. However, any thioesterase domain that releases an acyl amino acid produced by a peptide synthetase complex from the peptide synthetase complex may be used in accordance with the present disclosure. In some embodiments, thioesterase domains are characterized by the presence of the consensus sequence: [LIV]-{KG}-[LIVFY]-[LIVMST]-G-[HYWV]-S-{YAG}-G-[GSTAC](SEQ ID NO: 22), where square brackets (“[ ]”) indicate amino acids that are typically present at that position, and squiggly brackets (“{ }”) indicate amino acids that amino acids that are typically not present at that position. Those skilled in the art will be aware of methods to determine whether a give polypeptide domain is a thioesterase domain. In some embodiments, a thioesterase domain is or comprises a domain that is at least 70% or more including, e.g., at least 75%, at least 80%, at least 85%, at least 90%, at least 95% or more (and up 100%), identical at the amino acid level to that found in Bacillus subtilis's surfactin synthetase complex's SrfC subunit.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

The present disclosure, among other things, provides technologies relating to generation of acyl amino acids and/or to improving yield of desirable acyl amino acids.

In some embodiments, provided technologies are particularly useful for making an acyl amino acid composition that is low in and/or is substantially free of acyl amino acids that are hydroxylated and/or methylated at one or more of the ω-n positions (e.g., ω-1, ω-2, and/or ω-3 positions) of a fatty acid portion of an acyl amino acid. In some embodiments, provided technologies are particularly useful for making an acyl amino acid composition that is enriched in and/or is substantially entirely of acyl amino acids that are hydroxylated and/or methylated at one or more of the ω-n positions (e.g., ω-1, ω-2, and/or ω-3 positions) of a fatty acid portion of an acyl amino acid. In some embodiments involving acyl amino acids produced by technologies described and/or utilized herein, such acyl amino acids comprise a beta-hydroxyl group.

In some embodiments, the present disclosure provides insights that a fatty acid portion of acyl amino acids produced in microbial cells such as Bacillus cells, are typically modified, for example, by hydroxylation and/or methylation, and that generation of such modified acyl amino acids can be controlled by use of strain engineering. In particular, the present inventors have demonstrated that modulating the level and/or activity of one or more fatty acid modifying enzymes (e.g., one or more fatty acid hydroxylases) present in microbial cells can increase or decrease modification (e.g., hydroxylation) of a fatty acid portion of an acyl amino acid at one or more ω-n positions, where n≥1 (e.g., ω-1, ω-2, and/or ω-3 positions). In some embodiments, such constructs can be engineered to increase modifications (e.g., hydroxylation) of a fatty acid portion of an acyl amino acid at one or more ω-n positions, where n≥1 (e.g., ω-1, ω-2, and/or ω-3 positions). In some embodiments, such constructs can be engineered to decrease modifications (e.g., hydroxylation) of a fatty acid portion of an acyl amino acid at one or more ω-n positions, where n≥1 (e.g., ω-1, ω-2, and/or ω-3 positions). Thus, the present disclosure teaches engineering acyl amino acid-producing cells to comprise a modification (e.g., a genetic modification) that modulates modification (e.g., hydroxylation and/or alkylation such as methylation) of a fatty acid portion of an acyl amino acid. In some embodiments, the present disclosure teaches engineering acyl amino acid-producing cells to comprise a genetic modification such that expression and/or activity of a fatty acid modifying enzyme is modulated. Such technologies can be useful in increasing yield of surfactants and/or fatty acids of interest (e.g., with or without ω-n hydroxyl groups, where n≥1).

### I. Fatty Acid Synthesis

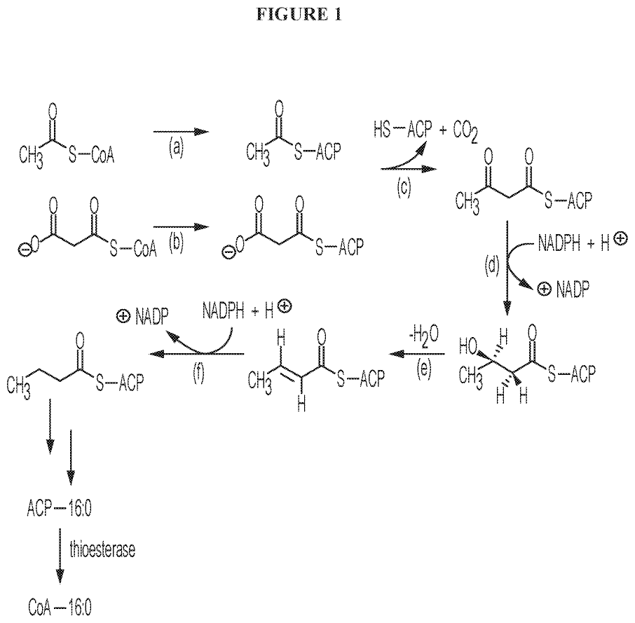

Those skilled in the art are aware that fatty acid synthesis involves serial extension of an initiator moiety through addition of acetyl moieties (i.e., —CH₂—C(O)—) transferred from a malonate entity onto the growing fatty acid chain. The process can be conceptualized as involving several steps; FIG. 1 provides a visual depiction of exemplary such steps, using an acetyl initiation moiety):
1. Loading: each of the initiation moiety and the malonyl group is loaded (see arrows labeled (a) and (b) in FIG. 1) onto carrier proteins, so that an initiation entity and the malonate entity are generated;
2. Condensation: a 2-carbon acetyl moiety is transferred from the malonyl entity onto the initiation moiety in a reaction that releases the initiation moiety from its association with its carrier protein and also releases CO₂ from the malonate (see arrow (c) in FIG. 1);
3. Carbonyl Reduction: NADPH is used to reduce the carbonyl of the initiation moiety to an —OH group (see arrow (d) in FIG. 1);
4. Dehydration: removal of H₂O removes the —OH group and generates a double bond (see arrow (e) in FIG. 1); and
5. Double Bond Reduction: in synthesis of saturated fatty acids, a second NADPH is used to reduce the generated double bond.
   Repeating these steps extends the fatty acid chain, each time by two carbons. The terminal carbon in the fatty acid chain, which terminal carbon is contributed by the initiation moiety, is known as the “omega” (o) carbon.

In the particular example depicted in FIG. 1, the initiation moiety is an acetyl moiety, which has two carbons, so that fatty acid chains synthesized on such an acetyl initiation moiety typically will have an even number of carbons. Those skilled in the art are aware that fatty acids can be synthesized on any of a variety of initiation moieties, so that either even-numbered or odd-numbered fatty acid chains can be generated.

Furthermore, those skilled in the art are aware that certain possible initiation moieties may include one or more branches (e.g., may contain one or more pendant alkyl moieties, such as one or more methyl, ethyl, propyl, butyl, pentyl, hexyl, heptyl, octyl, nonyl, decyl, undecyl, dodecyl, or longer, moieties, which themselves may be branched or linear). See, for example, Julotok et al. Appl. Environ Microbiol. 76:1423, March 2010, which includes certain “potential fatty acid precursors” (i.e., sources of initiation moieties as described herein), including as depicted in its FIG. 1, which includes certain branched-chain amino acids (e.g., isoleucine, valine, leucine), branched-chain α-keto acids (e.g., α-keto-methylvalerate, α-keto-isovalerate, α-keto-isocaproate), short-branched-chain carboxylic acids (e.g., 2-methylbutyrate, isobutyrate, isovalerate), branched-chain C6-fatty acid isomers (e.g., 2-ethylbutyrate, 2-methylpentanoate, 3-methylpentanoate), short-chain carboxylic acids (e.g., acetate, propionate, pyruvate, lactate, butyrate, sodium diacetate), medium-chain carboxylic acids, pentanoate, hexanoate, heptanoate, octanoate, decanoate).

Still further, those skilled in the art will be aware that many or most (or all) microbial cells include enzymes that select initiation moieties and/or associate them with an appropriate carrier protein. Technologies have been developed to modify such enzymes (see, for example, WO2014/144649 which, among other things, describes engineering microbes so that altered fatty acid branching patterns are achieved, e.g., through altered selection and/or relative utilization of certain branched vs linear initiation moieties; in some embodiments, cells are engineered so that their ability to synthesize and/or utilize one or more natural initiation moieties or entities is reduced or abolished so that, in some embodiments, selection and/or use of initiation moieties or entities is dependent upon feeding relevant precursor(s) to the cells). In some embodiments, the present disclosure may utilize (e.g., further engineer) microbial cells with such altered ability to synthesize, select, and/or utilize one or more particular initiation moieties or entities.

### II. Exemplary Engineered Cells for Generation of Acyl Amino Acids

In some aspects, provided are engineered cells that are capable of producing acyl amino acids and modulating hydroxylation and/or methylation of a fatty acid portion of such acyl amino acids. In some embodiments, such an engineered cell is an acyl amino acid-producing cell, which comprises a modification (e.g., a genetic modification) that modulates hydroxylation and/or methylation of a fatty acid portion of an acyl amino acid. In some embodiments, such an engineered cell expresses at least one peptide synthetase, which produces or synthesizes an acyl amino acid. In some embodiments, a cell that is engineered as described herein is one that contains one or more additional modifications relative to a reference (e.g., its parent) strain with respect to fatty acid synthesis. For example, in some embodiments a utilized cell may be one containing one or more modifications of fatty acid precursor metabolism, so that its ability to, and/or the frequency or efficiency with which it does, synthesize, select, and/or utilize a particular fatty acid precursor (e.g., fatty acid synthesis initiation moiety or entity) is altered relative to the reference strain. To give but a couple of examples, in some embodiments, a microbial cell that is engineered as described herein is one that lacks (e.g., has been engineered to lack, or otherwise lacks) one or more (e.g., all) α-keto acid dehydrogenase activities, and/or expresses a β-ketoacyl ACP synthase activity.

a. Modification of a Fatty Acid Portion of an Acyl Amino Acid

In some embodiments involving a modification (e.g., a genetic modification) that modulates hydroxylation and/or methylation of a fatty acid potion of an acyl amino acid, such a modification may result in an increase in the number of hydroxyl and/or alkyl (e.g., methyl) groups of a fatty acid portion (e.g., a fatty acid backbone portion) of an acyl amino acid by at least 10% or more, including, e.g., at least 20%, at least 30%, at least 40%, at least 50%, at least 60%, at least 70%, at least 80%, at least 90%, at least 95%, or more, as compared to that when the modification is absent. In alternative embodiments, such a modification (e.g., a genetic modification) may result in a reduction in the number of hydroxyl and/or alkyl (e.g., methyl) groups of a fatty acid portion (e.g., a fatty acid backbone portion) of an acyl amino acid by at least 10% or more, including, e.g., at least 20%, at least 30%, at least 40%, at least 50%, at least 60%, at least 70%, at least 80%, at least 90%, at least 95%, or more, as compared to that when the modification is absent.

In some embodiments, a modification (e.g., genetic modification) that modulates hydroxylation and/or methylation of a fatty acid portion (e.g., a fatty acid backbone portion) of an acyl amino acid is or comprises a genetic modification such that expression and/or activity of a fatty acid modifying enzyme is modulated. For example, in some embodiments, such a genetic modification may result in an increase in expression and/or activity of a fatty acid modifying enzyme by at least 10% or more, including, e.g., at least 20%, at least 30%, at least 40%, at least 50%, at least 60%, at least 70%, at least 80%, at least 90%, at least 95%, or more, as compared to that when the modification is absent. For example, in some embodiments, such a genetic modification may result in a reduction in expression and/or activity of a fatty acid modifying enzyme by at least 10% or more, including, e.g., at least 20%, at least 30%, at least 40%, at least 50%, at least 60%, at least 70%, at least 80%, at least 90%, at least 95%, or more, as compared to that when the modification is absent. In some embodiments, expression and/or activity of a fatty acid modifying enzyme may be modulated (e.g., increased or reduced) by modifying a gene that encodes a fatty acid modifying enzyme, which includes, e.g., but are not limited to a genetic modification (e.g., as described and/or utilized herein) of a gene sequence that encodes a fatty acid modifying enzyme. In some embodiments, expression and/or activity of a fatty acid modifying enzyme may be modulated (e.g., increased or reduced) by modifying one or more regulatory elements that are operably linked to a gene that encodes a fatty acid modifying enzyme, which includes, e.g., a genetic modification (e.g., as described and/or utilized herein) of one or more such regulatory elements.

In some embodiments, a fatty acid modifying enzyme may be endogenous to an acyl amino acid-producing cell to be engineered. In some embodiments, a fatty acid modifying enzyme may be heterologous to an acyl amino acid-producing cell to be engineered. In some embodiments, a fatty acid modifying enzyme may be or comprise a fatty acid modifying enzyme from a Bacillus cell (e.g., a Bacillus subtilis cell). In some embodiments, a fatty acid modifying enzyme may be or comprise one or more cytochrome P450 enzymes, for example, from a Bacillus cell (e.g., a Bacillus subtilis cell). Table 1 shows corresponding genes encoding eight cytochrome P450 enzymes present in a Bacillus cell (e.g., a Bacillus subtilis cell). See, e.g., “Respiratory Cytochromes, Other Heme Proteins, and Heme Biosynthesis.” Von Wachenfeldt and Hederstedt (2002) in Bacillus subtilis and its closest relatives, edited by Sonenshein, Hoch and Losick.

TABLE 1
Cytochrome P450 enzymes present in Bacillus cells
(e.g., Bacillus subtilis cells)

| Gene | Function |
|---|---|
| biol | Putative fatty acid hydroxylase |
| cyp107J1 | unknown |
| cyp134A1 | unknown |
| cyp109B1 | unknown |
| cyp152A1 | Fatty acid hydroxylase |
| cyp102A2 | Fatty acid hydroxylase |
| cyp102A3 | Fatty acid hydroxylase |
| cyp107K1 | unknown |

In some embodiments, a fatty acid modifying enzyme in which expression and/or activity is modulated may be encoded by a gene listed in Table 1 above.

In some embodiments, a fatty acid modifying enzyme, in which expression and/or activity is modulated, is encoded by CYP107H (also called BioI). In some embodiments, such an enzyme is typically involved in biotin synthesis and/or hydroxylates myristic acid. See, e.g., Cryle et al., “Products of cytochrome P450 (BioI) (CYP107H1)-catalyzed oxidation of fatty acids.” Org Lett. (2003) 5(18):3341-4. In some embodiments, an exemplary amino acid sequence of such a fatty acid modifying enzyme encoded by CYP107H is set forth in SEQ ID NO: 3 (GenBank accession number CAB14997.1) as provided in the section entitled “Listing of Certain Sequences” below.

In some embodiments, a fatty acid modifying enzyme, in which expression and/or activity is modulated, is encoded by CYP107J1 (also called CypA). In some embodiments, expression of such an enzyme can increase when one or more polychlorinated biphenyls are fed to Bacillus cells, e.g., Bacillus subtilis cells-BioI increases in expression as well. See, e.g., Sun et al. “Formation of hydroxylated and methoxylated polychlorinated biphenyls by Bacillus subtilis: New insights into microbial metabolism” Sci Total Environ. (2018) 613-614: 54-61. In some embodiments, an increase in expression of such an enzyme can correlate with hydroxylation of one or more polychlorinated biphenyls, followed by subsequent methylation to create O-methyl modified polychlorinated biphenyls. In some embodiments, an exemplary amino acid sequence of such a fatty acid modifying enzyme encoded by CYP107J1 (GenBank accession number CAB14615.1) is set forth in SEQ ID NO: 4 as provided in the section entitled “Listing of Certain Sequences” below.

In some embodiments, a fatty acid modifying enzyme, in which expression and/or activity is modulated, is encoded by CYP134A1 (also called cyclo-L-leucyl-L-leucyl dipeptide oxidase, pulcheriminic synthase). In some embodiments, such an enzyme can participate in production of pulcheriminic acid. See, e.g., Cryle et al. “Structural and biochemical characterization of the cytochrome P450 CypX (CYP134A1) from Bacillus subtilis: a cyclo-L-leucyl-L-leucyl dipeptide oxidase.” Biochemistry. (2010) 49(34):7282-96. In some embodiments, an exemplary amino acid sequence of such a fatty acid modifying enzyme encoded by CYP134A1 is set forth in SEQ ID NO: 7 (GenBank accession number CAB15511.1) as provided in the section entitled “Listing of Certain Sequences” below.

In some embodiments, a fatty acid modifying enzyme, in which expression and/or activity is modulated, is encoded by CYP109B1. In some embodiments, such an enzyme can hydroxylate both α- and β-ionone in vivo and in vitro. See, e.g., Zhang et al. “The crystal structure of the versatile cytochrome P450 enzyme CYP109B1 from Bacillus subtilis” Mol Biosyst. (2015) 11(3):869-81. In some embodiments, an exemplary amino acid sequence of such a fatty acid modifying enzyme encoded by CYP109B1 is set forth in SEQ ID NO: 6 (GenBank accession number CAB13078.1) as provided in the section entitled “Listing of Certain Sequences” below.

In some embodiments, a fatty acid modifying enzyme, in which expression and/or activity is modulated, is encoded by CYP152A1 (also called fatty acid b-hydroxylating cytochrome P450). In some embodiments, such an enzyme can catalyze hydrogen peroxide dependent hydroxylation of long chain fatty acids, producing α- and β-hydroxylated derivatives of myristic acid. See, e.g., Zhang et al. “The crystal structure of the versatile cytochrome P450 enzyme CYP109B1 from Bacillus subtilis” Mol Biosyst. (2015) 11(3):869-81. In some embodiments, an exemplary amino acid sequence of such a fatty acid modifying enzyme encoded by CYP152A1 is set forth in SEQ ID NO: 8 (GenBank accession number CAB12004.1) as provided in the section entitled “Listing of Certain Sequences” below.

In some embodiments, a fatty acid modifying enzyme, in which expression and/or activity is modulated, is encoded by CYP102A2. In some embodiments, CYP102A2 is a homolog of CYP102A3. See, e.g., Gustafsson et al. “Expression, purification, and characterization of Bacillus subtilis cytochromes P450 CYP102A2 and CYP102A3: flavocytochrome homologues of P450 BM3 from Bacillus megaterium” Biochemistry (2004) 43: 5474-87. In some embodiments, such an enzyme can hydroxylate myristic acid at the ω-1, ω-2 and ω-3 positions (e.g., 20%, 61% and 17%, respectively). In some embodiments, a fatty acid modifying enzyme encoded by CYP102A2 enzyme may prefer shorter unbranched fatty acids (e.g., lauric acid>myristic acid) and/or branched myristic acid. See, e.g., id. In some embodiments, CYP102A2 can hydroxylate surfactant sodium dodecyl sulphate (SDS). See, e.g., Axarli et al. “Cytochrome P450 102A2 Catalyzes Efficient Oxidation of Sodium Dodecyl Sulphate: A Molecular Tool for Remediation.” Enzyme Res. (2010) 2010:125429. In some embodiments, an exemplary amino acid sequence of such a fatty acid modifying enzyme encoded by CYP102A2 is set forth in SEQ ID NO: 1 (GenBank accession number CAB12544.1) as provided in the section entitled “Listing of Certain Sequences” below.

In some embodiments, a fatty acid modifying enzyme, in which expression and/or activity is modulated, is encoded by CYP102A3 (as known as cypB). In some embodiments, such an enzyme can hydroxylate myristic acid at the ω-1, ω-2 and ω-3 positions (e.g., 10%, 46% and 42%, respectively). In some embodiments, such an enzyme may prefer branched myristic acid relative to unbranched lauric or myristic acid. See, e.g., “Expression, purification, and characterization of Bacillus subtilis cytochromes P450 CYP102A2 and CYP102A3: flavocytochrome homologues of P450 BM3 from Bacillus megaterium” Biochemistry (2004) 43: 5474-87. In some embodiments, an exemplary amino acid sequence of such a fatty acid modifying enzyme encoded by CYP102A3 is set forth in SEQ ID NO: 2 (GenBank accession number CAB14658.1) as provided in the section entitled “Listing of Certain Sequences” below.

In some embodiments, a fatty acid modifying enzyme, in which expression and/or activity is modulated, is encoded by CYP107K1 (also known as polyketide biosynthesis cytochrome P450 PksS). In some embodiments, such an enzyme is involved in synthesis of bacillaene and the natural substrate of such an enzyme is or comprises dihydrobacillaene. In some embodiments, a CYP107K1 gene is located in the pksX cluster. In some embodiments, a CYP107K1 gene may be involved in reducing a particular double bond. See, e.g., Reddick et al. “PksS from Bacillus subtilis is a cytochrome P450 involved in bacillaene metabolism” Biochem Biophys Res Commun. (2007) 358(1):363-7. In some embodiments, an exemplary amino acid sequence of such a fatty acid modifying enzyme encoded by CYP107K1 is set forth in SEQ ID NO: 5 (GenBank accession number ABQ22962.1) as provided in the section entitled “Listing of Certain Sequences” below.

In some embodiments, a fatty acid modifying enzyme can be or comprise a fatty acid hydroxylase and/or a fatty acid methylase. In some embodiments, a fatty acid modifying enzyme is or comprises a fatty acid hydroxylase. In some such embodiments, a fatty acid hydroxylase may hydroxylate a linear or unbranched fatty acid, while in some such embodiments, a fatty acid hydroxylase may hydroxylate a branched fatty acid. In some embodiments, a fatty acid hydroxylase may be selected to hydroxylate a specific fatty acid, including, e.g., but not limited to caproic acid, caprylic acid, lauric acid, and myristic acid. In some embodiments, a fatty acid hydroxylase may be selected to hydroxylate a fatty acid as listed in Table 2 below. Examples of a fatty acid hydroxylase that may be involved in generation of acyl amino acids can be encoded by a gene selected from the group consisting of bioI, cyp107h, cyp107J1, cyp134A1, cyp109B1, cyp152A1, cyp102A2, cyp102A3, cyp107K1, and combinations thereof. Various fatty acid modifying enzymes (e.g., fatty acid hydroxylases and/or fatty acid methylases) may be involved in generation of different kinds of acyl amino acids, for example, depending on the types of the fatty acid and/or amino acid moieties of acyl amino acids to be generated. One of ordinary skill in the art reading the present disclosure will appreciate that a proper fatty acid modifying enzyme may be selected for modification to modulate hydroxylation and/or methylation of a particular fatty acid portion of an acyl amino acid accordingly.

b. Peptide Synthetases

In some embodiments, an acyl amino acid-producing cell to be engineered may comprise a peptide synthetase (e.g., ones as described and/or utilized herein). In some embodiments, such a peptide synthetase may be endogenous to a host cell. In some such embodiments, a peptide synthetase may be heterologous to a host cell. In some embodiments, a peptide synthetase may be an engineered peptide synthetase. In some embodiments, a cell (e.g., a microbial cell) can be engineered to express a peptide synthetase. For example, in some embodiments, a cell (e.g., a microbial cell) can be engineered to contain a polynucleotide encoding a peptide synthetase.

In some embodiments, a peptide synthetase comprises a fatty acid linkage domain, a peptide synthetase domain; and a thioesterase domain. In some embodiments, a peptide synthetase comprises a fatty acid linkage domain, a peptide synthetase domain, and a reductase domain. In some embodiments, a peptide synthetase may be a peptide synthetase complex as described in WO2008/131002, the contents of which are incorporated herein by reference in their entirety for the purposes described herein. In some embodiments, one or more of a fatty acid linkage domain, a peptide synthetase domain, a thioesterase domain, and a reductase domain (e.g., ones as described in WO2008/131002, the contents of which are incorporated herein by reference in their entirety for the purposes described herein) may be used in a peptide synthetase. In some embodiments, a fatty acid linkage domain for use in a peptide synthetase is or comprises a domain that is at least 70% or more, including, e.g., at least 75%, at least 80%, at least 85%, at least 90%, at least 95% or higher (and up to 100%), identical at the amino acid level to that found in Bacillus subtilis's surfactin synthetase SrfA protein. In some embodiments, a peptide synthetase domain for use in a peptide synthetase is or comprises a domain that is at least 70% or more, including, e.g., at least 75%, at least 80%, at least 85%, at least 90%, at least 95% or higher (and up to 100%), identical at the amino acid level to Bacillus subtilis's surfactin synthetase complex SrfA-A polypeptide subunit's first peptide synthetase domain. In some embodiments, a reductase domain for use in a peptide synthetase is or comprises a domain that is at least 70% or more, including, e.g., at least 75%, at least 80%, at least 85%, at least 90%, at least 95%, or more (and up to 100%), identical at the amino acid level to that found in Bacillus brevis's linear gramicidin synthetase complex. In some embodiments, a thioesterase domain is or comprises a domain that is at least 70% or more including, e.g., at least 75%, at least 80%, at least 85%, at least 90%, at least 95% or more (and up 100%), identical at the amino acid level to that found in Bacillus subtilis's surfactin synthetase complex's SrfC subunit.

In some embodiments, a peptide synthetase may be or comprise a single peptide synthetase domain, not associated (e.g., not associated covalently and/or not otherwise associated) with, for example, another domain typically found in a peptide synthetase complex (e.g., a fatty acid linkage domain, a thioesterase domain, a reductase domain, etc. and/or a combination thereof). In some embodiments, such a single peptide synthetase domain (e.g., as described in WO2014/144649, the contents of which are incorporated herein by reference in their entirety for the purposes described herein) may be used in a peptide synthetase.

In some embodiments, peptide synthetase domains useful for the production of acyl amino acids as described herein, correspond and/or show significant homology and/or identity to a first peptide synthetase domain found in a naturally-occurring peptide synthetase complex. That is, as is known in the art, some peptide synthetase domains (i.e., some polypeptides comprising adenylation (A), thiolation (T), and condensation (C) domains) catalyze condensation of a fatty acid with an amino acid, and some catalyze condensation of two amino acids with one another. In some embodiments, peptide synthetase domains useful for the production of acyl amino acids as described herein are those that catalyze condensation of an amino acid with a fatty acid; such peptide synthetase domains are typically utilized herein in a form (e.g., as part of a polypeptide) that is separated from and/or does not include another peptide synthetase domain.

Many naturally-occurring peptide synthetase domains are found in nature within peptide synthetase complexes that synthesize lipopeptides. Such peptide synthetase complexes are multi-enzymatic complexes found in both prokaryotes and eukaryotes, and comprising one or more enzymatic subunits that catalyze the non-ribosomal production of a variety of peptides (see, for example, Kleinkauf et al., Annu. Rev. Microbiol. 41:259-289, 1987; see also U.S. Pat. Nos. 5,652,116 and 5,795,738). Non-ribosomal synthesis is also known as thiotemplate synthesis (see e.g., Kleinkauf et al.). Peptide synthetase complexes typically include one or more peptide synthetase domains that recognize specific amino acids and are responsible for catalyzing addition of the amino acid to the polypeptide chain.

The catalytic steps in the addition of amino acids typically include: recognition of an amino acid by the peptide synthetase domain, activation of the amino acid (formation of an amino-acyladenylate), binding of the activated amino acid to the enzyme via a thioester bond between the carboxylic group of the amino acid and an SH group of an enzymatic co-factor, which cofactor is itself bound to the enzyme inside each peptide synthetase domain, and formation of the peptide bonds among the amino acids.

A peptide synthetase domain comprises subdomains that carry out specific roles in these steps to form the peptide product. One subdomain, the adenylation (A) domain, is responsible for selectively recognizing and activating the amino acid that is to be incorporated by a particular unit of the peptide synthetase. The activated amino acid is joined to the peptide synthetase through the enzymatic action of another subdomain, the thiolation (T) domain, that is generally located adjacent to the A domain. Amino acids joined to successive units of the peptide synthetase are subsequently linked together by the formation of amide bonds catalyzed by another subdomain, the condensation (C) domain.

Peptide synthetase domains that catalyze the addition of D-amino acids often also have the ability to catalyze the recemization of L-amino acids to D-amino acids. Peptide synthetase complexes also typically include a conserved thioesterase domain that terminates the growing amino acid chain and releases the product.

The genes that encode peptide synthetase complexes have a modular structure that parallels the functional domain structure of the complexes (see, for example, Cosmina et al., Mol. Microbiol. 8:821, 1993; Kratzxchmar et al., J. Bacteriol. 171:5422, 1989; Weckermann et al., Nuc. Acids res. 16:11841, 1988; Smith et al., EMBO J. 9:741, 1990; Smith et al., EMBO J. 9:2743, 1990; MacCabe et al., J. Biol. Chem. 266:12646, 1991; Coque et al., Mol. Microbiol. 5:1125, 1991; Diez et al., J. Biol. Chem. 265:16358, 1990).

Hundreds of peptides are known to be produced by peptide synthetase complexes. Such nonribosomally-produced peptides often have non-linear structures, including cyclic structures exemplified by the peptides surfactin, cyclosporin, tyrocidin, and mycobacillin, or branched cyclic structures exemplified by the peptides polymyxin and bacitracin. Moreover, such nonribosomally-produced peptides may contain amino acids not usually present in ribosomally-produced polypeptides such as for example norleucine, beta-alanine and/or ornithine, as well as D-amino acids. Additionally or alternatively, such nonribosomally-produced peptides may comprise one or more non-peptide moieties that are covalently linked to the peptide. As one non-limiting example, surfactin is a cyclic lipopeptide that comprises a beta-hydroxy fatty acid covalently linked to the first glutamate of the lipopeptide. Other non-peptide moieties that are covalently linked to peptides produced by peptide synthetase complexes are known to those skilled in the art, including for example sugars, chlorine or other halogen groups, N-methyl and N-formyl groups, glycosyl groups, acetyl groups, etc.

Typically, each amino acid of the non ribosomally-produced peptide is specified by a distinct peptide synthetase domain. For example, the surfactin synthetase complex which catalyzes the polymerization of the lipopeptide surfactin consists of three enzymatic subunits. The first two subunits each comprise three peptide synthetase domains, whereas the third has only one. These seven peptide synthetase domains are responsible for the recognition, activation, binding and polymerization of L-Glu, L-Leu, D-Leu, L-Val, L-Asp, D-Leu and L-Leu, the amino acids present in surfactin.

A similar organization in discrete, repeated peptide synthetase domains occurs in various peptide synthetase genes in a variety of species, including bacteria and fungi, for example srfA (Cosmina et al., Mol. Microbiol. 8, 821-831, 1993), grsA and grsB (Kratzxchmar et al., J. Bacterial. 171, 5422-5429, 1989) tycA and tycB (Weckermann et al., Nucl. Acid. Res. 16, 11841-11843, 1988) and ACV from various fungal species (Smith et al., EMBO J. 9, 741-747, 1990; Smith et al., EMBO J. 9, 2743-2750, 1990; MacCabe et al., J. Biol. Chem. 266, 12646-12654, 1991; Coque et al., Mol. Microbiol. 5, 1125-1133, 1991; Diez et al., J. Biol. Chem. 265, 16358-16365, 1990). The peptide synthetase domains of even distant species contain sequence regions with high homology, some of which are conserved and specific for all the peptide synthetases. Additionally, certain sequence regions within peptide synthetase domains are even more highly conserved among peptide synthetase domains which recognize the same amino acid (Cosmina et al., Mol. Microbiol. 8, 821-831, 1992).

Exemplary lipopeptides synthesized by peptide synthetase complexes in nature are listed below in Table 2 (See also the NORINE database, which provides access to information on peptides and lipopeptides that are known to be, or in some cases believed to be, produced by peptide synthetase enzymes; still further, see Segolene et al. (Segolene et al., “NORINE: a database of nonribosomal peptides.” Nucleic Acids Research, 36: D327-D331, 2008.)).

TABLE 2
Exemplary Lipopeptides Synthesized by Peptide Synthetases

| Lipopeptide | Fatty Acid | Fatty Acid |
|---|---|---|
| Name | Component | Component name |
| [Ala4]surfactin | aC15:0-OH(3) | 3-hydroxy-12-methyl- |
| aC15 | | tetradecanoic acid |
| [Ala4]surfactin | iC14:0-OH(3) | 3-hydroxy-12-methyl- |
| iC14 | | tridecanoic acid |
| [Ala4]surfactin | iC15:0-OH(3) | 3-hydroxy-13-methyl- |
| iC15 | | tetradecanoic acid |
| [Ala4]surfactin | C14:0-OH(3) | 3-hydroxy-tetradecanoic acid |
| nC14 | | |
| [Ala4]surfactin | C15:0-OH(3) | 3-hydroxy-pentadecanoic acid |
| nC15 | | |
| [Gln1]surfactin | C15:0-OH(3) | 3-hydroxy-pentadecanoic acid |
| [Gln1]surfactin | aC15:0-OH(3) | 3-hydroxy-12-methyl- |
| aC15 | | tetradecanoic acid |
| [Gln1]surfactin | iC15:0-OH(3) | 3-hydroxy-13-methyl- |
| iC15 | | tetradecanoic acid |
| [Ile2.4.7]surfactin | aC15:0-OH(3) | 3-hydroxy-12-methyl- |
| | | tetradecanoic acid |
| [Ile4.7]surfactin | aC15:0-OH(3) | 3-hydroxy-12-methyl- |
| | | tetradecanoic acid |
| [Ile4]surfactin | aC15:0-OH(3) | 3-hydroxy-12-methyl- |
| | | tetradecanoic acid |
| [Ile7]surfactin | aC15:0-OH(3) | 3-hydroxy-12-methyl- |
| | | tetradecanoic acid |
| [Leu4]surfactin | aC15:0-OH(3) | 3-hydroxy-12-methyl- |
| | | tetradecanoic acid |
| [Phe25] | C10:0-OH(3) | 3-hydroxy-decanoic acid |
| syringopeptin 25A | | |
| [Val7]surfactin | aC15:0-OH(3) | 3-hydroxy-12-methyl- |
| | | tetradecanoic acid |
| A21978C1 | aC11:0 | 8-methyldecanoic acid |
| A21978C2 | iC12:0 | 10-methylundecanoic acid |
| A21978C3 | aC13:0 | 10-methyldodecanoic acid |
| A54145 A | iC10:0 | decanoic acid |
| A54145 A1 | C10:0 | decanoic acid |
| A54145 B | C10:0 | decanoic acid |
| A54145 B1 | iC10:0 | decanoic acid |
| A54145 C | aC11:0 | 8-methyldecanoic acid |
| A54145 D | aC11:0 | 8-methyldecanoic acid |
| A54145 E | aC11:0 | 8-methyldecanoic acid |
| A54145 F | iC10:0 | decanoic acid |
| amphibactin B | C14:0-OH(3) | 3-hydroxy-tetradecanoic acid |
| amphibactin C | C16:1(9)-OH(3) | 3-hydroxy-9-hexadecenoic acid |
| amphibactin D | C14:0 | tetradecanoic acid |
| amphibactin E | C16:1(9) | 9-hexadecenoic acid |
| amphibactin F | C16:0-OH(3) | 3-hydroxy-hexadecanoic acid |
| amphibactin G | C18:1(9)-OH(3) | 3-hydroxy-9-octadecenoic acid |
| amphibactin H | C16:0 | hexadecanoic acid |
| amphibactin I | C18:1(9) | 9-octadecenoic acid |
| amphisin | C10:0-OH(3) | 3-hydroxy-decanoic acid |
| amphomycin | iC13:1(3) | 11-methyl-3-dodecenoic acid |
| A1437 A | | |
| amphomycin | iC14:1(3) | 12-methyl-3-tridecenoic acid |
| A1437 B | | |
| amphomycin | aC15:1(3) | 12-methyl-3-tetradecenoic acid |
| A1437 D | | |
| amphomycin | aC13:1(3) | 10-methyl-3-dodecenoic acid |
| A1437 E | | |
| apramide A | C8:0:1(7)-Me(2) | 2-methylact-7-ynoic acid |
| apramide B | C8:0:1(7) | oct-7-ynoic acid |
| apramide C | C9:1(8)-Me(2) | 2-methyl-8-noneic acid |
| apramide D | C8:0:1(7)-Me(2) | 2-methylact-7-ynoic acid |
| apramide E | C8:0:1(7) | oct-7-ynoic acid |
| apramide F | C9:1(8)-Me(2) | 2-methyl-8-noneic acid |
| apramide G | C8:0:1(7)-Me(2) | 2-methylact-7-ynoic acid |
| aquachelin A | C12:1(5) | 2-methyl-5-dodecenoic acid |
| aquachelin B | C12:0 | dodecanoic acid |
| aquachelin C | C14:1(7) | 7-tetradecenoic acid |
| aquachelin D | C14:0 | tetradecanoic acid |
| arthrofactin | C10:0-OH(3) | 3-hydroxy-decanoic acid |
| arylomycin A1 | iC11:0 | 9-methyldecanoic acid |
| arylomycin A2 | iC12:0 | 10-methylundecanoic acid |
| arylomycin A3 | C12:0 | dodecanoic acid |
| arylomycin A4 | aC13:0 | 10-methyldodecanoic acid |
| arylomycin A5 | iC14:0 | 12-methyl-tridecanoic acid |
| arylomycin B1 | iC11:0 | 9-methyldecanoic acid |
| arylomycin B2 | iC12:0 | 10-methylundecanoic acid |
| arylomycin B3 | C12:0 | dodecanoic acid |
| arylomycin B4 | aC13:0 | 10-methyldodecanoic acid |
| arylomycin B5 | iC13:0 | 11-methyldodecanoic acid |
| arylomycin B6 | iC14:0 | 12-methyl-tridecanoic acid |
| arylomycin B7 | aC15:0 | 12-methyltetradecanoic acid |
| bacillomycin D-1 | C14:0-NH2(3) | 3-amino-tetradecanoic acid |
| bacillomycin D-2 | iC15:0-NH2(3) | 3-amino-13-methyl- |
| | | tetradecanoic acid |
| bacillomycin D-3 | aC15:0-NH2(3) | 3-amino-12-methyl-tetradecanoic |
| | | acid |
| bacillomycin D-4 | C16:0-NH2(3) | 3-amino-hexadecanoic acid |
| bacillomycin D-5 | iC16:0-NH2(3) | 3-amino-14-methyl- |
| | | pentadecanoic acid |
| bacillomycin F-1 | iC15:0-NH2(3) | 3-amino-13-methyl-tetradecanoic |
| | | acid |
| bacillomycin F-2 | aC15:0-NH2(3) | 3-amino-12-methyl-tetradecanoic |
| | | acid |
| bacillomycin F-3 | iC16:0-NH2(3) | 3-amino-14-methyl- |
| | | pentadecanoic acid |
| bacillomycin F-4 | C16:0-NH2(3) | 3-amino-hexadecanoic acid |
| bacillomycin F-5 | iC17:0-NH2(3) | 3-amino-15-methyl-hexadecanoic |
| | | acid |
| bacillomycin F-6 | aC17:0-NH2(3) | 3-amino-14-methyl-hexadecanoic |
| | | acid |
| bacillomycin L-1 | C14:0-NH2(3) | 3-amino-tetradecanoic acid |
| bacillomycin L-2 | iC15:0-NH2(3) | 3-amino-13-methyl-tetradecanoic |
| | | acid |
| bacillomycin L-3 | aC15:0-NH2(3) | 3-amino-12-methyl-tetradecanoic |
| | | acid |
| bacillomycin L-4 | C16:0-NH2(3) | 3-amino-hexadecanoic acid |
| bacillomycin L-5 | iC16:0-NH2(3) | 3-amino-14-methyl- |
| | | pentadecanoic acid |
| beauverolide A | C10:0-Me(4)- | 3-hydroxy-4-methyl-decanoic |
| | OH(3) | acid |
| beauverolide B | C10:0-Me(4)- | 3-hydroxy-4-methyl-decanoic |
| | OH(3) | acid |
| beauverolide Ba | C10:0-Me(4)- | 3-hydroxy-4-methyl-decanoic |
| | OH(3) | acid |
| beauverolide C | C10:0-Me(4)- | 3-hydroxy-4-methyl-decanoic |
| | OH(3) | acid |
| beauverolide Ca | C10:0-Me(4)- | 3-hydroxy-4-methyl-decanoic |
| | OH(3) | acid |
| beauverolide D | C8:0-Me(4)- | 4-methyl-3-hydroxy-octanoic |
| | OH(3) | acid |
| beauverolide E | C8:0-Me(4)- | 4-methyl-3-hydroxy-octanoic |
| | OH(3) | acid |
| beauverolide Ea | C8:0-Me(4)- | 4-methyl-3-hydroxy-octanoic |
| | OH(3) | acid |
| beauverolide F | C8:0-Me(4)- | 4-methyl-3-hydroxy-octanoic |
| | OH(3) | acid |
| beauverolide Fa | C8:0-Me(4)- | 4-methyl-3-hydroxy-octanoic |
| | OH(3) | acid |
| beauverolide H | C9:0-OH(3) | 3-hydroxy-nonanoic acid |
| beauverolide I | C8:0-Me(4)- | 4-methyl-3-hydroxy-octanoic |
| | OH(3) | acid |
| beauverolide II | C10:0-Me(4)- | 3-hydroxy-4-methyl-decanoic |
| | OH(3) | acid |
| beauverolide III | C8:0-Me(4)- | 4-methyl-3-hydroxy-octanoic |
| | OH(3) | acid |
| beauverolide IV | C8:0-Me(4)- | 4-methyl-3-hydroxy-octanoic |
| | OH(3) | acid |
| beauverolide Ja | C8:0-Me(4)- | 4-methyl-3-hydroxy-octanoic |
| | OH(3) | acid |
| beauverolide Ka | C10:0-Me(4)- | 3-hydroxy-4-methyl-decanoic |
| | OH(3) | acid |
| beauverolide L | C10:0-Me(4)- | 3-hydroxy-4-methyl-decanoic |
| | OH(3) | acid |
| beauverolide La | C10:0-Me(4)- | 3-hydroxy-4-methyl-decanoic |
| | OH(3) | acid |
| beauverolide M | C8:0-Me(4)- | 4-methyl-3-hydroxy-octanoic |
| | OH(3) | acid |
| beauverolide N | C8:0-Me(4)- | 4-methyl-3-hydroxy-octanoic |
| | OH(3) | acid |
| beauverolide V | C8:0-Me(4)- | 4-methyl-3-hydroxy-octanoic |
| | OH(3) | acid |
| beauverolide VI | C8:0-Me(4)- | 4-methyl-3-hydroxy-octanoic |
| | OH(3) | acid |
| beauverolide VII | C8:0-Me(4)- | 4-methyl-3-hydroxy-octanoic |
| | OH(3) | acid |
| beauverolide VIII | C10:0-Me(4)- | 3-hydroxy-4-methyl-decanoic |
| | OH(3) | acid |
| callipeltin A | iC8:0-Me(2.4)- | 2,4,6-trimethyl-3-hydroxy- |
| | OH(3) | heptanoic acid |
| callipeltin C | iC8:0-Me(2.4)- | 2,4,6-trimethyl-3-hydroxy- |
| | OH(3) | heptanoic acid |
| callipeltin D | iC8:0-Me(2.4)- | 2,4,6-trimethyl-3-hydroxy- |
| | OH(3) | heptanoic acid |
| callipeltin F | iC8:0-Me(2.4)- | 2,4,6-trimethyl-3-hydroxy- |
| | OH(3) | heptanoic acid |
| callipeltin G | iC8:0-Me(2.4)- | 2,4,6-trimethyl-3-hydroxy- |
| | OH(3) | heptanoic acid |
| callipeltin H | iC8:0-Me(2.4)- | 2,4,6-trimethyl-3-hydroxy- |
| | OH(3) | heptanoic acid |
| callipeltin I | iC8:0-Me(2.4)- | 2,4,6-trimethyl-3-hydroxy- |
| | OH(3) | heptanoic acid |
| callipeltin J | iC8:0-Me(2.4)- | 2,4,6-trimethyl-3-hydroxy- |
| | OH(3) | heptanoic acid |
| callipeltin K | iC8:0-Me(2.4)- | 2,4,6-trimethyl-3-hydroxy- |
| | OH(3) | heptanoic acid |
| callipeltin L | iC8:0-Me(2.4)- | 2,4,6-trimethyl-3-hydroxy- |
| | OH(3) | heptanoic acid |
| carmabin A | C10:0:1(9)- | 2,4-dimethyl-dec-9-ynoic acid |
| | Me(2.4) | |
| carmabin B | C10:0-Me(2.4)- | 9-oxo-2,4- |
| | oxo(9) | dimethyldecanoic acid |
| CDA1b | C6:0-Ep(2) | 2-epoxy-hexanoic acid |
| CDA2a | C6:0-Ep(2) | 2-epoxy-hexanoic acid |
| CDA2b | C6:0-Ep(2) | 2-epoxy-hexanoic acid |
| CDA2d | C6:0-Ep(2) | 2-epoxy-hexanoic acid |
| CDA2fa | C6:0-Ep(2) | 2-epoxy-hexanoic acid |
| CDA2fb | C6:0-Ep(2) | 2-epoxy-hexanoic acid |
| CDA3a | C6:0-Ep(2) | 2-epoxy-hexanoic acid |
| CDA3b | C6:0-Ep(2) | 2-epoxy-hexanoic acid |
| CDA4a | C6:0-Ep(2) | 2-epoxy-hexanoic acid |
| CDA4b | C6:0-Ep(2) | 2-epoxy-hexanoic acid |
| cormycin A | C16:0-OH(3.4) | 3,4-dihydroxy- |
| | | hexadecanoic acid |
| corpeptin A | C10:0-OH(3) | 3-hydroxy-decanoic acid |
| corpeptin B | C12:1(5)-OH(3) | 3-hydroxy-5-dodecenoic acid |
| corrugatin | C8:0 | octanoic acid |
| daptomycin | C10:0 | decanoic acid |
| enduracidin A | iC12:2(2.t4) | 10-methyl-2,trans4-undecanoic |
| | | acid |
| enduracidin B | aC13:2(2.t4) | 10-methyl-2,trans4-dodecenoic |
| | | acid |
| fengycin A | C16:0-OH(3) | 3-hydroxy-hexadecanoic acid |
| fengycin B | C16:0-OH(3) | 3-hydroxy-hexadecanoic acid |
| friulimicin A | iC13:1(3) | 11-methyl-3-dodecenoic acid |
| friulimicin B | iC14:1(3) | 12-methyl-3-tridecenoic acid |
| friulimicin C | aC13:1(3) | 10-methyl-3-dodecenoic acid |
| friulimicin D | aC15:1(3) | 12-methyl-3-tetradecenoic acid |
| fuscopeptin A | C8:0-OH(3) | 3-hydroxy-octanoic acid |
| fuscopeptin B | C10:0-OH(3) | 3-hydroxy-decanoic acid |
| Ile-polymyxin B1 | aC9:0 | 6-methyloctanoic acid |
| Ile-polymyxin E1 | aC9:0 | 6-methyloctanoic acid |
| Ile-polymyxin E2 | iC8:0 | 6-methylheptanoic acid |
| Ile-polymyxin E8 | aC10:0 | 8-methyldecanoic acid |
| iturin A-1 | C13:0-NH2(3) | 3-amino-tridecanoic acid |
| iturin A-2 | C14:0-NH2(3) | 3-amino-tetradecanoic acid |
| iturin A-3 | aC15:0-NH2(3) | 3-amino-12-methyl-tetradecanoic |
| | | acid |
| iturin A-4 | iC15:0-NH2(3) | 3-amino-13-methyl-tetradecanoic |
| | | acid |
| iturin A-5 | C15:0-NH2(3) | 3-amino-pentadecanoic acid |
| iturin A-6 | iC16:0-NH2(3) | 3-amino-14-methyl- |
| | | pentadecanoic acid |
| iturin A-7 | C16:0-NH2(3) | 3-amino-hexadecanoic acid |
| iturin A-8 | aC17:0-NH2(3) | 3-amino-14-methyl-hexadecanoic |
| | | acid |
| iturin C-1 | iC14:0-NH2(3) | 3-amino-12-methyl-tridecanoic |
| | | acid |
| iturin C-2 | aC15:0-NH2(3) | 3-amino-12-methyl-tetradecanoic |
| | | acid |
| iturin C-3 | iC16:0-NH2(3) | 3-amino-14-methyl- |
| | | pentadecanoic acid |
| iturin C-4 | aC17:0-NH2(3) | 3-amino-14-methyl-hexadecanoic |
| | | acid |
| kulomo opunalide 1 | C8:0:1(7)- | 2-methyl-3-hydroxy-7-octynoic |
| | Me(2)-OH(3) | acid |
| kulomo opunalide 2 | C8:0:1(7)- | 2-methyl-3-hydroxy-7-octynoic |
| | Me(2)-OH(3) | acid |
| lichenysin A aC13 | aC13:0-OH(3) | 3-hydroxy-10-methyl-dodecanoic |
| | | acid |
| lichenysin A aC15 | aC15:0-OH(3) | 3-hydroxy-12-methyl- |
| | | tetradecanoic acid |
| lichenysin A aC17 | aC17:0-OH(3) | 3-hydroxy-14-methyl- |
| | | hexadecanoic acid |
| lichenysin A iC12 | iC12:0-OH(3) | 3-hydroxy-10-methyl-undecanoic |
| | | acid |
| lichenysin A iC13 | iC13:0-OH(3) | 3-hydroxy-11-methyl-dodecanoic |
| | | acid |
| lichenysin A iC14 | iC14:0-OH(3) | 3-hydroxy-12-methyl-tridecanoic |
| | | acid |
| lichenysin A iC15 | iC15:0-OH(3) | 3-hydroxy-13-methyl- |
| | | tetradecanoic acid |
| lichenysin A iC16 | iC16:0-OH(3) | 3-hydroxy-14-methyl- |
| | | pentadecanoic acid |
| lichenysin A iC17 | iC17:0-OH(3) | 3-hydroxy-15-methyl- |
| | | hexadecanoic acid |
| lichenysin A nC12 | C12:0-OH(3) | 3-hydroxy-dodecanoic acid |
| lichenysin A nC13 | C13:0-OH(3) | 3-hydroxy-tridecanoic acid |
| lichenysin A nC14 | C14:0-OH(3) | 3-hydroxy-tetradecanoic acid |
| lichenysin A nC15 | C15:0-OH(3) | 3-hydroxy-pentadecanoic acid |
| lichenysin A nC16 | C16:0-OH(3) | 3-hydroxy-hexadecanoic acid |
| lokisin | C10:0-OH(3) | 3-hydroxy-decanoic acid |
| marinobactin A | C12:0 | dodecanoic acid |
| marinobactin B | C14:1(7) | 7-tetradecenoic acid |
| marinobactin C | C14:0 | tetradecanoic acid |
| marinobactin D1 | C16:1(9) | 9-hexadecenoic acid |
| marinobactin D2 | C16:1(7) | 7-hexadecenoic acid |
| marinobactin E | C16:0 | hexadecanoic acid |
| massetolide A | C10:0-OH(3) | 3-hydroxy-decanoic acid |
| massetolide B | C11:0-OH(3) | 3-hydroxy-undecanoic acid |
| massetolide C | C12:0-OH(3) | 3-hydroxy-dodecanoic acid |
| massetolide D | C10:0-OH(3) | 3-hydroxy-decanoic acid |
| massetolide E | C10:0-OH(3) | 3-hydroxy-decanoic acid |
| massetolide F | C10:0-OH(3) | 3-hydroxy-decanoic acid |
| massetolide G | C11:0-OH(3) | 3-hydroxy-undecanoic acid |
| massetolide H | C12:0-OH(3) | 3-hydroxy-dodecanoic acid |
| massetolide L | C10:0-OH(3) | 3-hydroxy-decanoic acid |
| mycosubtilin 1 | C16:0-NH2(3) | 3-amino-hexadecanoic acid |
| mycosubtilin 2 | iC16:0-NH2(3) | 3-amino-14-methyl- |
| | | pentadecanoic acid |
| mycosubtilin 3 | iC17:0-NH2(3) | 3-amino-15-methyl-hexadecanoic |
| | | acid |
| mycosubtilin 4 | aC17:0-NH2(3) | 3-amino-14-methyl-hexadecanoic |
| | | acid |
| neamphamide A | iC8:0-Me(2.4)- | 2,4,6-trimethyl-3-hydroxy- |
| | OH(3) | heptanoic acid |
| Nva-polymyxin E1 | aC9:0 | 6-methyloctanoic acid |
| papuamide A | aC11:2(4.6)- | 2,3-dihydroxy-2,6,8- |
| | Me(2.6)- | trimethyldeca-(4Z,6E)-dienoic |
| | OH(2.3) | acid |
| papuamide B | aC11:2(4.6)- | 2,3-dihydroxy-2,6,8- |
| | Me(2.6)- | trimethyldeca-(4Z,6E)-dienoic |
| | OH(2.3) | acid |
| papuamide C | aC11:2(4.6)- | 2,3-dihydroxy-2,6,8- |
| | Me(2.6)- | trimethyldeca-(4Z,6E)-dienoic |
| | OH(2.3) | acid |
| papuamide D | aC11:2(4.6)- | 2,3-dihydroxy-2,6,8- |
| | Me(2.6)- | trimethyldeca-(4Z,6E)-dienoic |
| | OH(2.3) | acid |
| pholipeptin | C10:0-OH(3) | 3-hydroxy-decanoic acid |
| plusbacin A1 | C14:0-OH(3) | 3-hydroxy-tetradecanoic acid |
| plusbacin A2 | iC15:0-OH(3) | 3-hydroxy-13-methyl- |
| | | tetradecanoic acid |
| plusbacin A3 | iC16:0-OH(3) | 3-hydroxy-14-methyl- |
| | | pentadecanoic acid |
| plusbacin A4 | C16:0-OH(3) | 3-hydroxy-hexadecanoic acid |
| plusbacin B1 | C14:0-OH(3) | 3-hydroxy-tetradecanoic acid |
| plusbacin B2 | iC15:0-OH(3) | 3-hydroxy-13-methyl- |
| | | tetradecanoic acid |
| plusbacin B3 | iC16:0-OH(3) | 3-hydroxy-14-methyl- |
| | | pentadecanoic acid |
| plusbacin B4 | C16:0-OH(3) | 3-hydroxy-hexadecanoic acid |
| polymyxin B1 | aC9:0 | 6-methyloctanoic acid |
| polymyxin B2 | iC8:0 | 6-methylheptanoic acid |
| polymyxin B3 | C8:0 | octanoic acid |
| polymyxin B4 | C7:0 | heptanoic acid |
| polymyxin B5 | C9:0 | nonanoic acid |
| polymyxin B6 | aC9:0-OH(3) | 3-hydroxy-6-methyloctanoic acid |
| polymyxin E1 | aC9:0 | 6-methyloctanoic acid |
| polymyxin E2 | iC8:0 | 6-methylheptanoic acid |
| polymyxin E3 | C8:0 | octanoic acid |
| polymyxin E4 | C7:0 | heptanoic acid |
| polymyxin E7 | iC9:0 | 7-methyloctanoic acid |
| polymyxin M | aC9:0 | 6-methyloctanoic acid |
| pseudomycin A | C14:0-OH(3.4) | 3,4-dihydroxy-tetradecanoic acid |
| pseudomycin B | C14:0-OH(3) | 3-hydroxy-tetradecanoic acid |
| pseudomycin C | C16:0-OH(3.4) | 3,4-dihydroxy-hexadecanoic acid |
| pseudomycin C2 | C16:0-OH(3) | 3-hydroxy-hexadecanoic acid |
| pseudophomin A | C10:0-OH(3) | 3-hydroxy-decanoic acid |
| pseudophomin B | C12:0-OH(3) | 3-hydroxy-dodecanoic acid |
| putisolvin I | C6:0 | hexanoic acid |
| putisolvin II | C6:0 | hexanoic acid |
| putisolvin III | C6:0 | hexanoic acid |
| ramoplanin A1 | C8:2(2.t4) | 2,trans4-octenoic acid |
| ramoplanin A2 | iC9:2(2.t4) | 2,trans4-7-methyl-octenoic acid |
| ramoplanin A3 | iC10:2(2.t4) | 2,trans4-8-methyl-noneoic acid |
| serrawettin W1 | C10:0-OH(3) | 3-hydroxy-decanoic acid |
| serrawettin W2 | C10:0-OH(3) | 3-hydroxy-decanoic acid |
| surfactin aC13 | aC13:0-OH(3) | 3-hydroxy-10-methyl-dodecanoic |
| | | acid |
| surfactin aC15 | aC15:0-OH(3) | 3-hydroxy-12-methyl- |
| | | tetradecanoic acid |
| surfactin iC12 | iC12:0-OH(3) | 3-hydroxy-10-methyl-undecanoic |
| | | acid |
| surfactin iC14 | iC14:0-OH(3) | 3-hydroxy-12-methyl-tridecanoic |
| | | acid |
| surfactin iC15 | iC15:0-OH(3) | 3-hydroxy-13-methyl- |
| | | tetradecanoic acid |
| surfactin iC16 | iC16:0-OH(3) | 3-hydroxy-14-methyl- |
| | | pentadecanoic acid |
| surfactin nC13 | C13:0-OH(3) | 3-hydroxy-tridecanoic acid |
| surfactin nC14 | C14:0-OH(3) | 3-hydroxy-tetradecanoic acid |
| surfactin nC15 | C15:0-OH(3) | 3-hydroxy-pentadecanoic acid |
| syringafactin A | C10:0-OH(3) | 3-hydroxy-decanoic acid |
| syringafactin B | C10:0-OH(3) | 3-hydroxy-decanoic acid |
| syringafactin C | C10:0-OH(3) | 3-hydroxy-decanoic acid |
| syringafactin D | C12:0-OH(3) | 3-hydroxy-dodecanoic acid |
| syringafactin E | C12:0-OH(3) | 3-hydroxy-dodecanoic acid |
| syringafactin F | C12:0-OH(3) | 3-hydroxy-dodecanoic acid |
| syringomycin A1 | C10:0-OH(3) | 3-hydroxy-decanoic acid |
| syringomycin E | C12:0-OH(3) | 3-hydroxy-dodecanoic acid |
| syringomycin G | C14:0-OH(3) | 3-hydroxy-tetradecanoic acid |
| syringopeptin 22 | C10:0-OH(3) | 3-hydroxy-decanoic acid |
| PhvA | | |
| syringopeptin 22 | C12:0-OH(3) | 3-hydroxy-dodecanoic acid |
| PhvB | | |
| syringopeptin 22A | C10:0-OH(3) | 3-hydroxy-decanoic acid |
| syringopeptin 22B | C12:0-OH(3) | 3-hydroxy-dodecanoic acid |
| syringopeptin 25A | C10:0-OH(3) | 3-hydroxy-decanoic acid |
| syringopeptin 25B | C12:0-OH(3) | 3-hydroxy-dodecanoic acid |
| syringopeptin | C12:0-OH(3) | 3-hydroxy-dodecanoic acid |
| 508A | | |
| syringopeptin | C14:0-OH(3) | 3-hydroxy-tetradecanoic acid |
| 508B | | |
| syringopeptin SC 1 | C10:0-OH(3) | 3-hydroxy-decanoic acid |
| syringopeptin SC 2 | C12:0-OH(3) | 3-hydroxy-dodecanoic acid |
| syringostatin A | C14:0-OH(3) | 3-hydroxy-tetradecanoic acid |
| syringostatin B | C14:0-OH(3.4) | 3,4-dihydroxy-tetradecanoic acid |
| syringotoxin B | C14:0-OH(3) | 3-hydroxy-tetradecanoic acid |
| tensin | C10:0-OH(3) | 3-hydroxy-decanoic acid |
| tolaasin A | Pda | pentanedioic acid |
| tolaasin B | C8:0-OH(3) | 3-hydroxy-octanoic acid |
| tolaasin C | C8:0-OH(3) | 3-hydroxy-octanoic acid |
| tolaasin D | C8:0-OH(3) | 3-hydroxy-octanoic acid |
| tolaasin E | C8:0-OH(3) | 3-hydroxy-octanoic acid |
| tolaasin I | C8:0-OH(3) | 3-hydroxy-octanoic acid |
| tolaasin II | C8:0-OH(3) | 3-hydroxy-octanoic acid |
| tripropeptin A | iC13:0-OH(3) | 3-hydroxy-11-methyl-dodecanoic |
| | | acid |
| tripropeptin B | iC14:0-OH(3) | 3-hydroxy-12-methyl-tridecanoic |
| | | acid |
| tripropeptin C | iC15:0-OH(3) | 3-hydroxy-13-methyl- |
| | | tetradecanoic acid |
| tripropeptin D | iC16:0-OH(3) | 3-hydroxy-14-methyl- |
| | | pentadecanoic acid |
| tripropeptin E | iC17:0-OH(3) | 3-hydroxy-15-methyl- |
| | | hexadecanoic acid |
| tripropeptin Z | iC12:0-OH(3) | 3-hydroxy-10-methyl-undecanoic |
| | | acid |
| Val-polymyxin E1 | aC9:0 | 6-methyloctanoic acid |
| Val-polymyxin E2 | iC8:0 | 6-methylheptanoic acid |
| viscosin | C10:0-OH(3) | 3-hydroxy-decanoic acid |
| viscosinamide | C10:0-OH(3) | 3-hydroxy-decanoic acid |
| White Line | C10:0-OH(3) | 3-hydroxy-decanoic acid |
| Inducing Principle | | |

Non-limiting examples of peptide synthetase complexes that may contain peptide synthetase domains useful in the identification, selection, design, and/or production of engineered peptide synthetases as described herein include, for example, surfactin synthetase, fengycin synthetase, arthrofactin synthetase, lichenysin synthetase, syringomycin synthetase, syringopeptin synthetase, saframycin synthetase, gramicidin synthetase, cyclosporin synthetase, tyrocidin synthetase, mycobacillin synthetase, polymyxin synthetase, bacitracin synthetase, and combinations thereof.

In some embodiments, an engineered peptide synthetase, peptide synthetase domain, or component thereof contains insertions, deletions, substitutions or inversions of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or more amino acids as compared to its relevant reference.

In certain embodiments, such amino acid substitutions result in a peptide synthetase that comprises an amino acid whose side chain contains a structurally similar side chain as compared to the corresponding amino acid in the relevant reference. For example, amino acids with aliphatic side chains, including glycine, alanine, valine, leucine, and isoleucine, may be substituted for each other; amino acids having aliphatic-hydroxyl side chains, including serine and threonine, may be substituted for each other; amino acids having amide-containing side chains, including asparagine and glutamine, may be substituted for each other; amino acids having aromatic side chains, including phenylalanine, tyrosine, and tryptophan, may be substituted for each other; amino acids having basic side chains, including lysine, arginine, and histidine, may be substituted for each other; and amino acids having sulfur-containing side chains, including cysteine and methionine, may be substituted for each other.

In certain embodiments, amino acid substitutions result in a peptide synthetase that comprises an amino acid whose side chain exhibits similar chemical properties to a corresponding amino acid present in a relevant reference. For example, in certain embodiments, amino acids that comprise hydrophobic side chains may be substituted for each other. In some embodiments, amino acids may be substituted for each other if their side chains are of similar molecular weight or bulk. For example, an amino acid in a peptide synthetase may be substituted for an amino acid present in the relevant reference if its side chains exhibits a minimum/maximum molecular weight or takes up a minimum/maximum amount of space.

In certain embodiments, a peptide synthetase shows at least about 50, 55, 60, 65, 70, 75, 80, 85, 90, 91, 92, 93, 94, 95, 96, 97, 98, or 99% homology or identity with a relevant reference (e.g., over a portion that spans at least 5, 6, 7, 8, 9, 10, 15, 20, 25, 30, 35, 40, 45, 50, 60, 70, 80, 90, 100, 200, 300, 400, 500, 600, 700, 800, 900, 1000 or more amino acids).

c. Host Cells

In some embodiments, engineered polypeptides (e.g., an engineered polypeptide for modulation of hydroxylation and/or methylation of a fatty acid portion of an acyl amino acid, and/or an engineered peptide synthetase) are introduced in host cells for the production of acyl amino acids. As will be understood by those skilled in the art, in some embodiments, such engineered polypeptides are typically introduced into a host cell using an expression vector. Those skilled in the art reading the present disclosure will appreciate that various host cells may be used, and in some embodiments, a host cell is capable of receiving and propagating such an expression vector, and is capable of expressing the engineered polypeptide. In some embodiments, a host cell may be capable of producing an acyl amino acid. In some such embodiments, a host cell has been engineered to produce an acyl amino acid. An engineered polypeptide (e.g., an engineered polypeptide for modulation of hydroxylation and/or methylation of a fatty acid portion of an acyl amino acid, and/or an engineered peptide synthetase) may be transiently or stably introduced into a host cell of interest. For example, an engineered polypeptide (e.g., an engineered polypeptide for modulation of hydroxylation and/or methylation of a fatty acid portion of an acyl amino acid, and/or an engineered peptide synthetase) may be stably introduced by integrating the engineered polypeptide into the chromosome of a host cell. Additionally or alternatively, an engineered polypeptide (e.g., an engineered polypeptide for modulation of hydroxylation and/or methylation of a fatty acid portion of an acyl amino acid, and/or an engineered peptide synthetase) may be transiently introduced by introducing a vector comprising the engineered polypeptide into a host cell, which vector is not integrated into the genome of the host cell.

In certain embodiments, a host cell to be engineered is a microbial cell. In certain embodiments, a host cell to be engineered is a bacterium. Non-limiting examples of bacteria that are useful as host cells for technologies described and/or utilized herein include bacteria of the genera Escherichia, Streptococcus, Bacillus, and a variety of other genera known to those skilled in the art. In certain embodiments, an engineered polypeptide (e.g., an engineered polypeptide for modulation of hydroxylation and/or methylation of a fatty acid portion of an acyl amino acid, and/or an engineered peptide synthetase) is introduced into a host cell of the species Bacillus subtilis.

In some embodiments, microbial host cells (e.g., bacterial host cells) may be wild type. In some embodiments, microbial host cells (e.g., bacterial host cells) may comprise one or more genetic changes as compared to wild type species. In certain embodiments, such genetic changes are useful to production of acyl amino acids in a host cell. For example, such genetic changes may various advantages useful in production of acyl amino acids (e.g., increased viability, ability to utilize alternative energy sources, ability to make different acyl amino acids etc.).

In certain embodiments, a host cell to be engineered is a plant cell. Those skilled in the art are aware of standard techniques for introducing one or more engineered polypeptides (e.g., an engineered polypeptide such as ones described herein for modulation of hydroxylation and/or methylation of a fatty acid portion of an acyl amino acid, and/or an engineered peptide synthetase such as ones as described herein) into a plant cell of interest such as, without limitation, gold bombardment and agrobacterium transformation. In certain embodiments, the present disclosure provides a transgenic plant that comprises an engineered polypeptide (e.g., ones as described herein) that produces an acyl amino acid of interest (e.g., an acyl amino acid with or without a hydroxyl group at one or more ω-n C positions, where n≥1 (e.g., ω-1, ω-2, and/or ω-3 positions) of a fatty acid portion of the acyl amino acid). Any of a variety of plants species may be made transgenic by introduction of one or more engineered polypeptides (e.g., an engineered polypeptide such as ones described herein for modulation of hydroxylation and/or methylation of a fatty acid portion of an acyl amino acid, and/or an engineered peptide synthetase such as ones as described herein), such that the one or more engineered polypeptides are expressed in the plant and produces an acyl amino acid of interest (e.g., an acyl amino acid with or without a hydroxyl group at one or more ω-n C positions (e.g., ω-1, ω-2, and/or ω-3 positions) of a fatty acid portion of the acyl amino acid). In some embodiments, such one or more engineered polypeptides (e.g., ones as described herein) may be expressed in transgenic plants systemically (e.g. in each tissue at all times) or only in localized tissues and/or during certain periods of time. Those skilled in the art will be aware of various promoters, enhancers, etc. that may be employed to control when and where one or more engineered polypeptides (e.g., ones as described herein) are expressed.

Insects, including insects that are threats to agriculture crops, can produce acyl amino acids that are likely to be important or essential for insect physiology. For example, an enzyme related to peptide synthetases produces the product of the Drosophila Ebony genes, which product is important for proper pigmentation of the fly, but is also important for proper function of the nervous system (see e.g., Richardt et al., Ebony, a novel nonribosomal peptide synthetase for beta-alanine conjugation with biogenic amines in Drosophila, J. Biol. Chem., 278(42):41160-6, 2003). Acyl amino acids are also produced by certain Lepidoptera species that are a threat to crops. Thus, technologies provided herein may be used to produce transgenic plants that produce an acyl amino acid of interest that kills such insects or otherwise disrupts their adverse effects on crops. For example, an engineered polypeptide that produces an acyl amino acid that is toxic to a given insect species may be introduced into a plant such that insects that infest such a plant are killed. Additionally or alternatively, an engineered polypeptide that produces an acyl amino acid that disrupts an essential activity of the insect (e.g., feeding, mating, etc.) may be introduced into a plant such that the commercially adverse effects of insect infestation are minimized or eliminated. In certain embodiments, an acyl amino acid that mitigates an insect's adverse effects on a plant is an acyl amino acid that is naturally produced by such an insect. In certain embodiments, an acyl amino acid that mitigates an insect's adverse effects on a plant is a structural analog of an acyl amino acid that is naturally produced by such an insect. In some embodiments, technologies provided herein can be useful in allowing the construction of engineered polypeptides that produce any of a variety of acyl amino acids, which acyl amino acids can be used in controlling or eliminating harmful insect infestation of one or more plant species.

### III. Exemplary Compositions

Technologies described and/or utilized herein are useful for making an acyl amino acid composition and/or a fatty acid composition. Thus, in some embodiments, the present disclosure provides engineered cells and/or methods of using such cells to produce compositions as described herein (e.g., that are or comprise particular acyl amino acids and/or fatty acids, optionally together with or isolated from one or more bacterial components and/or one or more enzymatic components).

a. Acyl Amino Acid Compositions and Methods of Generating and/or Isolating the Same

Some aspects of the present disclosure provides compositions comprising acyl amino acids produced by engineered cells (e.g., ones as described herein). In some embodiments, such compositions comprise a collection of individual acyl amino acid molecules, that are related to one another in that they are each synthesized by provided engineered cells and together represent a distribution of chemical entities, varied in precise chemical structure (e.g., due to varying length and/or composition of acyl chains, linkages within such acyl chains and/or between an acyl chain and the amino acid, etc), that are synthesized by certain relevant peptide synthetase(s), under the conditions of synthesis (e.g., in vivo or in vitro). In some embodiments, a provided composition includes straight-chain acyl moieties, branched acyl moieties, and/or combinations thereof.

In some embodiments where an acyl amino acid composition is produced by engineered cells (e.g., ones as described herein) comprising a modification (e.g., a genetic modification) such that expression and/or activity of a fatty acid modifying enzyme (e.g., a fatty acid hydroxylase) is reduced and thereby reduce hydroxylation of a fatty acid portion of an acyl amino acid (e.g., reduce hydroxylation of carbon at one or more Cω-n position, where n≥1 (e.g., ω-1, ω-2, and/or ω-3 positions) of a fatty acid portion of an acyl amino acid), less than 10%, less than 7.5%, less than 5%, less than 2.5%, less than 1%, less than 0.5%, less than 0.2%, or less than 0.1% of a fatty acid portion of an acyl amino acid in an acyl amino acid composition is hydroxylated (e.g., at one or more ω-n positons, where n≥1 (e.g., ω-1, ω-2, and/or ω-3 positions) of the fatty acid portion of the acyl amino acid), as compared to an acyl amino composition made using cells without such a modification. In some such embodiments, an acyl amino acid composition is substantially free of acyl amino acids that are hydroxylated at one or more ω-n positions, where n≥1 (e.g., ω-1, ω-2, and/or ω-3 positon) of the fatty acid portion of the acyl amino acid. In some embodiments, at least a fraction (e.g., at least 10% or more, including, e.g., at least 20%, at least 30%, at least 40%, at least 50%, at least 60%, at least 70%, at least 80%, at least 90%, at least 95%, or more and up to 100%) of acyl amino acid in such compositions provided herein may have a beta-hydroxyl group.

In some embodiments where an acyl amino acid composition is produced by engineered cells (e.g., ones as described herein) comprising a modification (e.g., a genetic modification) such that expression and/or activity of a fatty acid modifying enzyme (e.g., a fatty acid methylase) is reduced and thereby reduce methylation of a fatty acid portion of an acyl amino acid (e.g., reduce methylation of carbon at one or more Cω-n position, where n≥1 (e.g., Co-1, ω-2, and/or ω-3 positions) of a fatty acid portion of an acyl amino acid), less than 10%, less than 7.5%, less than 5%, less than 2.5%, less than 1%, less than 0.5%, less than 0.2%, or less than 0.1% of a fatty acid portion of an acyl amino acid in an acyl amino acid composition is methylated (e.g., at one or more ω-n positons, where n≥1 (e.g., ω-1, ω-2, and/or ω-3 positions) of the fatty acid portion of the acyl amino acid), as compared to an acyl amino composition made using cells without such a modification. In some such embodiments, an acyl amino acid composition is substantially free of acyl amino acids that are methylated at one or more ω-n positions, where n≥1 (e.g., ω-1, ω-2, and/or ω-3 positon) of the fatty acid portion of the acyl amino acid. In some embodiments, at least a fraction (e.g., at least 10% or more, including, e.g., at least 20%, at least 30%, at least 40%, at least 50%, at least 60%, at least 70%, at least 80%, at least 90%, at least 95%, or more and up to 100%) of acyl amino acid in such compositions provided herein may have a beta-hydroxyl group.

In some embodiments where an acyl amino acid composition is produced by engineered cells (e.g., ones as described herein) comprising a modification (e.g., a genetic modification) such that expression and/or activity of a fatty acid modifying enzyme (e.g., a fatty acid hydroxylase) is increased and thereby increase hydroxylation of a fatty acid portion of an acyl amino acid (e.g., increase hydroxylation of carbon at one or more Cω-n position, where n≥1 (e.g., ω-1, ω-2, and/or ω-3 positions) of a fatty acid portion of an acyl amino acid), greater than 60%, including, e.g., greater than 70%, greater than 80%, greater than 90%, greater than 95%, greater than 98%, or greater than 99% (and up ω-100%) of a fatty acid portion of an acyl amino acid in an acyl amino acid composition is hydroxylated (e.g., at one or more ω-n positons, where n≥1 (e.g., ω-1, ω-2, and/or ω-3 positions) of the fatty acid portion of the acyl amino acid), as compared to an acyl amino composition made using cells without such a modification. In some such embodiments, an acyl amino acid composition is substantially entirely of acyl amino acids that are hydroxylated at one or more ω-n positions, where n≥1 (e.g., ω-1, ω-2, and/or ω-3 positon) of the fatty acid portion of the acyl amino acid. In some such embodiments, at least a fraction (e.g., at least 10% or more, including, e.g., at least 20%, at least 30%, at least 40%, at least 50%, at least 60%, at least 70%, at least 80%, at least 90%, at least 95%, or more and up to 100%) of acyl amino acid in such compositions provided herein may have a beta-hydroxyl group.

In some embodiments where an acyl amino acid composition is produced by engineered cells (e.g., ones as described herein) comprising a modification (e.g., a genetic modification) such that expression and/or activity of a fatty acid modifying enzyme (e.g., a fatty acid methylase) is increased and thereby increase methylation of a fatty acid portion of an acyl amino acid (e.g., increase methylation of carbon at one or more ω-n position, where n≥1 (e.g., ω-1, ω-2, and/or ω-3 positions) of a fatty acid portion of an acyl amino acid), greater than 60%, including, e.g., greater than 70%, greater than 80%, greater than 90%, greater than 95%, greater than 98%, or greater than 99% (and up to ˜100%) of a fatty acid portion of an acyl amino acid in an acyl amino acid composition is methylated (e.g., at one or more ω-n positons, where n≥1 (e.g., ω-1, ω-2, and/or ω-3 positions) of the fatty acid portion of the acyl amino acid), as compared to an acyl amino composition made using cells without such a modification. In some such embodiments, an acyl amino acid composition is substantially entirely of acyl amino acids that are methylated at one or more ω-n positions, where n≥1 (e.g., ω-1, ω-2, and/or ω-3 positon) of the fatty acid portion of the acyl amino acid. In some such embodiments, at least a fraction (e.g., at least 10% or more, including, e.g., at least 20%, at least 30%, at least 40%, at least 50%, at least 60%, at least 70%, at least 80%, at least 90%, at least 95%, or more and up to 100%) of acyl amino acid in such compositions provided herein may have a beta-hydroxyl group.

It will be appreciated by those skilled in the art that, in some embodiments, one feature of engineered production of acyl amino acids is that such engineered cells (e.g., as described herein) may not generate pure populations of single chemical entities. Thus, as noted above, in some embodiments, provided herein are acyl amino acid compositions comprising distributions of chemical entities. In some embodiments, provided herein are acyl amino acid compositions in which substantially all acyl amino acids comprise the same amino acid moiety, but the composition includes a distribution of acyl moieties. For examples, in some embodiments, an acyl amino acid composition produced by technologies described herein may comprise a plurality of distinct acyl amino acids, each having the same amino acid moiety, but is enriched in one or a few (e.g., 1, 2, or 3) acyl amino acid (e.g., of a certain carbon length of a fatty acid portion), as compared to a reference product (e.g., an acyl amino acid composition that is not produced by technologies described herein). In some such embodiments, an acyl amino acid composition produced by technologies described herein may contain a higher proportion of an acyl amino acid having a particular carbon length of a fatty acid portion (e.g., C14 for a fatty acid portion) than that of a reference product (e.g., an acyl amino acid composition that is not produced by technologies described herein), for example, by at least 10%, including, e.g., at least 20%, at least 30%, at least 40%, at least 50%, at least 60%, at least 70%, at least 80%, at least 90%, or more. In some embodiments, an acyl amino acid composition produced by technologies described herein may contain at least 70% or more (including, e.g., at least 80%, at least 90%, at least 95% or more) of an acyl amino acid having a particular carbon length of a fatty acid portion (e.g., C14 for a fatty acid portion), based on the total acyl amino acid molecules present in the composition.

Technologies provided herein can be used to produce a wide variety of acyl amino acids and compositions. In some embodiments, the amino acid moiety of acyl amino acids and compositions is or comprises one found in an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and/or valine. Alternatively or additionally, in some embodiments, the amino acid moiety of acyl amino acids and compositions is or comprises one found in an amino acid selected from the group consisting of selenocysteine and/or pyrrolysine. In some embodiments, the amino acid moiety of acyl amino acids and compositions is or comprises one found in an amino acid selected from the group consisting of norleucine, beta-alanine and/or ornithine. In some embodiments, the amino acid moiety of acyl amino acids and compositions is or comprises one found in an amino acid selected from the group consisting of L-amino acids. In some embodiments, the amino acid moiety of acyl amino acids and compositions is or comprises one found in an amino acid selected from the group consisting of D-amino acids. In some embodiments, the amino acid moiety of acyl amino acids and compositions is or comprises or comprises one found in an amino acid D-glu or D-diaminopropionic acid. Those skilled in the art will be aware of appropriate amino acid substrates, usable by acyl amino acid-producing cells as described herein (and, for example, by engineered peptide synthetases as described herein) to generate acyl amino acids containing such amino acid moieties. In some embodiments, the amino acid substrate is or comprises the recited amino acid. In some embodiments, the acyl moiety (or the fatty acid moiety) of acyl amino acids and compositions is or comprises a saturated fatty acid such as butryic acid, caproic acid, caprylic acid, capric acid, lauric acid, myristic acid, palmitic acid, stearic arachidic acid, behenic acid, and/or lignoceric acid. In some embodiments, the acyl moiety (or the fatty acid moiety) of acyl amino acids and compositions is or comprises an unsaturated fatty acids such as, without limitation, myristoleic acid, palmitoleic acid, oliec acid, linoleic acid, alpha-linolenic acid, arachidonic acid, eicosapentaenoic acid, erucic acid, and/or docosahexaenoic acid. Other saturated and unsaturated fatty acids whose acyl moieties may be used in accordance with the present disclosure. In certain embodiments, acyl amino acids and compositions produced by technologies provided herein may comprise beta-hydroxy fatty acids as the acyl moiety (or fatty acid moiety) of acyl amino acid. As is understood by those of ordinary skill in the art, beta-hydroxy fatty acids comprise a hydroxy group attached to the R carbon of the fatty acid chain.

In some embodiments, the present disclosure provides acyl amino acids and compositions in which the acyl group comprises or consists of fatty acid chains with a length within a range bounded by a shorter length selected from the group consisting of C2, C3, C4, C5, C6, C7, C8, C9, C10, C11, C12, C13, C14, C15, C16, C17, C18, C19, C20, C21, C22, C23, C24, C25, C26, C27, C28, C29, C30, and an upper length selected from the group consisting of C30, C29, C28, C27, C26, C25, C24, C23, C22, C21, C20, C19, C18, C17, C16, C15, C14, C13, C12, C11, C10, C9, C8, C7, C6, C5, C4, C3, C2, and C1, wherein the upper length is the same as or larger than the lower length. In some particular embodiments, the present disclosure provides acyl amino acids and compositions in which the acyl group comprises or consists of C10-C14 fatty acid chains, C13-16 fatty acid chains, C13-15 fatty acid chains, C16-24 fatty acid chains, C18-22 fatty acid chains, C18-24 fatty acid chains, C8-C16 fatty acid chains. In some embodiments, the present disclosure provides acyl amino acids and compositions in which the acyl group comprises, consists predominantly of, or consists of C5, C6, C7, C8, C9, C10, C11, C12, C13, C14, C15, C16, C17, C18, C19, and/or C20 fatty acid chains. In some embodiments, the present disclosure provides acyl amino acids and compositions in which the acyl group comprises, consists predominantly of, or consists of comprises, consists predominantly of, or consists of C8, C9, C10, C11, C12, C13, C14, C15, and/or C16 fatty acid chains. In some embodiments, the present disclosure provides acyl amino acids and compositions in which the acyl group comprises, consists predominantly of, or consists of comprises, consists predominantly of, or consists of C12, C13, C14, C15, and/or C16 fatty acid chains.

In some embodiments, the present disclosure provides acyl amino acid compositions in which all acyl amino acids comprise the same amino acid moiety or comprise an amino acid moiety from the same amino acid.

In some embodiments, the present disclosure provides acyl amino acid compositions in which different acyl amino acids within the composition have different acyl moieties (e.g., acyl moieties that differ, in composition, structure, branching, and/or length (of one or more chains). In some embodiments, such compositions predominantly include acyl moieties of a length (or within a range of lengths) as set forth above. In some such embodiments, such predominant acyl moieties are present in the composition at a level of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 8100, 82%, 83%, 84%, 85%, 86%, 87%, 88%, 89%, 90%, 91%, 92%, 93%, 94%, 95%, 96%, 97%<98%, or 99%.

In certain embodiments, technologies described and/or provided herein are useful in large-scale production of acyl amino acids. In certain embodiments, acyl amino acids are produced in commercially viable quantities using engineered cells, compositions, and/or methods as described and/or utilized herein. For example, engineered cells (e.g., ones as described and/or utilized herein) may be used to produce acyl amino acids to a level of at least 5, 6, 7, 8, 9, 10, 11, 12, 13, 14, 15, 16, 17, 18, 19, 20, 30, 40, 50, 60, 70, 80, 90, 100, 110, 120, 130, 140, 150, 150, 200, 250, 300, 400, 500, 600, 700, 800, 900, 1000 mg/L or higher. As will be appreciated by those skilled in the art, biological production of acyl amino acids using technologies provided herein achieves certain advantages over other methods of producing acyl amino acids. For example, as compared to chemical production methods, production of acyl amino acids using technologies provided herein may reduce downstream purification, which, for example, can increase yield of desirable products. In some embodiments, production of acyl amino acids using technologies provided herein may utilize more readily available and starting materials that are easier to store, reduce the necessity of using harsh and sometimes dangerous chemical reagents in the manufacturing process, reduce the difficulty and efficiency of the synthesis itself by utilizing host cells as bioreactors, and/or reduce the fiscal and environmental cost of disposing of chemical by-products. Other advantages will be clear to practitioners who utilize technologies provided herein.

Accordingly, some aspects of the present disclosure provide methods of making an acyl amino acid composition using such engineered cells. In some embodiments, such a method comprises a step of (a) culturing an engineered cell (e.g., ones as described and/or utilized herein) under conditions and for a time sufficient for an acyl amino acid composition to be made.

In some embodiments involving culturing, such culturing can comprise incubating an engineered cell (e.g., ones as described and/or utilized herein) in a culture medium that comprises one or more of a carbon source, a fatty acid, and an amino acid. In some embodiments, such a culture medium may comprise a carbon source, a fatty acid, and an amino acid. In some embodiments involving making an acyl glycinate composition, an amino acid present in a culture medium is or comprises glycine. In some embodiments involving making an acyl glutamate composition, an amino acid present in a culture medium is or comprises glutamic acid. In some embodiments involving making an acyl sarcosinate composition, an amino acid present in a culture medium is or comprises sarcosine. One of ordinary skill in the art reading the present disclosure will appreciate that other amino acids can be present in a culture medium for use in making other acyl amino acids.

In some embodiments, engineered cells and/or methods described and/or utilized herein produce acyl amino acid compositions that contain at least 50% or more, including, e.g., at least 60%, at least 70%, at least 80%, at least 90%, at least 95%, or more, of acyl amino acid molecules having a linear or straight-chain fatty acid portion.

In some embodiments, engineered cells and/or methods described and/or utilized herein produce acyl amino acid compositions that contain at least 50% or more, including, e.g., at least 60%, at least 70%, at least 80%, at least 90%, at least 95%, or more, of acyl amino acid molecules having a branched fatty acid portion. For example, in some embodiments where engineered cells (e.g., ones as described herein) comprising a genetic modification that reduces expression and/or activity of α-keto acid dehydrogenase (e.g., by knocking out bkdAA and bkdAB) are cultured in a culture medium comprising particular organic acids to initiate fatty acid synthesis, acyl amino acid with different branching patterns may be obtained by varying an organic acid substrate that is fed to such engineered cells. In some embodiments, when such engineered cells are cultured in a culture medium comprising isobutyrate, at least 50% or more, including, e.g., at least 60%, at least 70%, at least 80%, at least 90%, at least 95%, or more, of acyl amino acid molecules in a composition have an iso-branched fatty acid portion. In some such embodiments, at least 50% or more, including, e.g., at least 60%, at least 70%, at least 80%, at least 90%, at least 95%, or more, of acyl amino acid molecules in a composition may have a fatty acid chain length of about 10-20, or about 12-18, or about 13-15. In some such embodiments, at least 50% or more, including, e.g., at least 60%, at least 70%, at least 80%, at least 90%, at least 95%, or more, of acyl amino acid molecules in a composition have a fatty acid chain length of 14.

In some embodiments, when such engineered cells (e.g., comprising a genetic modification that reduces expression and/or activity of α-keto acid dehydrogenase (e.g., by knocking out bkdAA and bkdAB)) are cultured in a culture medium comprising 2-methylburic acid or 2-methylbutyrate, at least 50% or more, including, e.g., at least 60%, at least 70%, at least 80%, at least 90%, at least 95%, or more, of acyl amino acid molecules in a composition have a fatty acid portion with anteiso-branching. In some such embodiments, at least 50% or more, including, e.g., at least 60%, at least 70%, at least 80%, at least 90%, at least 95%, or more, of acyl amino acid molecules in a composition may have an odd-numbered chain length, e.g., in some embodiments, a fatty acid chain length of 11, 13, 15, 17, or 19. In some such embodiments, at least 50% or more, including, e.g., at least 60%, at least 70%, at least 80%, at least 90%, at least 95%, or more, of acyl amino acid molecules in a composition may have a fatty acid chain length of 13 or 15.

In some embodiments, when such engineered cells (e.g., comprising a genetic modification that reduces expression and/or activity of α-keto acid dehydrogenase (e.g., by knocking out bkdAA and bkdAB)) are cultured in a culture medium comprising isovaleric acid or isovalerate, at least 50% or more, including, e.g., at least 60%, at least 70%, at least 80%, at least 90%, at least 95%, or more, of acyl amino acid molecules in a composition have a fatty acid portion with iso-branching. In some such embodiments, at least 50% or more, including, e.g., at least 60%, at least 70%, at least 80%, at least 90%, at least 95%, or more, of acyl amino acid molecules in a composition may have an odd-numbered chain length, e.g., in some embodiments, a fatty acid chain length of 11, 13, 15, 17, or 19. In some such embodiments, at least 50% or more, including, e.g., at least 60%, at least 70%, at least 80%, at least 90%, at least 95%, or more, of acyl amino acid molecules in a composition may have a fatty acid chain length of 13 or 15.

In some embodiments, acyl amino acids are produced in association with one or more components of a cell and/or with a peptide synthetase. In some embodiments, acyl amino acid compositions are subjected to one or more isolation procedures, for example as is known in the art, e.g., to separate produced acyl amino acid compounds from one or more components of their production system (e.g., from a peptide synthetase or component or domain thereof, and/or from one or more components of a cell such as an engineered cell.

b. Generation of Fatty Acids from Acyl Amino Acids

In some embodiments, acyl amino acids produced by technologies provided herein can be cleaved to generate free fatty acids (e.g., branched or linear fatty acids, and/or hydroxylated fatty acids) and amino acids. For example, in some embodiments, acyl amino acids produced by technologies provided herein can be treated with an acid and/or heat to break the amide bond that links the fatty acid moiety to the amino acid moiety, thereby generating free fatty acids and amino acids. In some embodiments, acyl amino acids produced by technologies provided herein can be cleaved to generate free fatty acid molecules and amino acid molecules using acylases. Exemplary methods for generating fatty acids from acyl amino acids are described in WO 2017/011592, the contents of which are incorporated herein by reference in its entirety for the purposes described herein. Those skilled in the art will appreciate that acyl amino acids produced by technologies provided herein can be cleaved to generate fatty acids using methods known in the art, e.g., as described in Examples 3 and 4 of WO 2017/011592.

Such free fatty acids generated from acyl amino acids can be used in various applications, e.g., directly as personal care products, and/or subject to further processing to make derivative products, such as surfactants that have new head groups different from the head group (amino acid) that was connected to the fatty acid in which it was originally produced.

c. Characterization, Formulation, and/or Incorporation of Acyl Amino Acid and/or Fatty Acids

Those skilled in the art, reading the present disclosure, will appreciate that acyl amino acids, fatty acids, and/or other entities generated in accordance with the present disclosure may be characterized and/or assessed for one or more attributes (e.g., desirable attributes), including for suitability in one or more particular commercial applications. Acyl amino acids can be assessed for their usefulness as surfactants, therapeutics (e.g., signaling molecules), anti-microbials, preservatives, anti-wrinkle agents, anti-acne agents and skin moisturizing agents, and other uses.

Those skilled in the art, reading the present disclosure will further appreciate that, in some embodiments, compositions as provided herein (e.g., that are or comprise acyl amino acids, fatty acids, and/or engineered cells or components thereof, etc.) can be utilized in particular commercial contexts, included by being formulated and/or otherwise incorporated into products such as, for example, acyl amino acids in personal care product, home care products, cosmetics, detergents, fabric softeners, oil field, agricultural and food products, and in the production and care of textiles; and fatty acids for use as polyols for manufacturing of polyurethanes and other polymers, anti-corrosives, lubricants, polishes, textile additives, soaps, shaving products, emollients and hydrocarbon fuels.

### EXEMPLIFICATION

### Example 1: Characterization of Acyl Amino Acids Produced by Existing Fermentation Methods

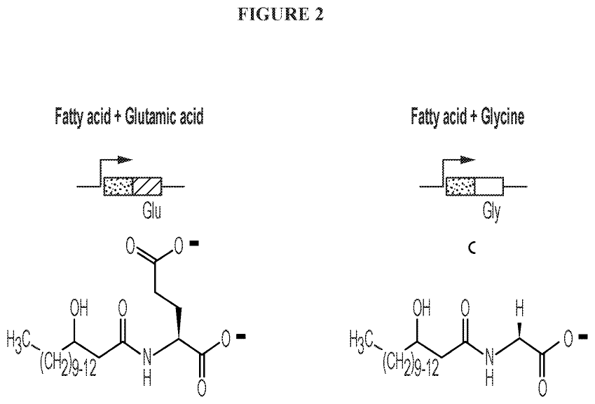

The present inventors have previously engineered peptide synthetase enzymes to produce acyl amino acid surfactants. See, e.g., Reznik et al., Appl Microbiol Biotechnol (2010) 86(5): 1387-97. The present inventors have found that a significant fraction of acyl glutamate (e.g., one having a structure as shown in FIG. 2) produced by previously-engineered peptide synthetase enzymes (see, for example, as shown in FIG. 2) has a mass of about 16 Dalton greater than the expected mass (approximately 300.42 Dalton), as determined by liquid chromatograph-mass spectrometry (LC-MS), which is commonly to measure the quantity of acyl amino acids produced by engineered microbial strains. The additional mass of about 16 Dalton suggests that a hydrogen of an acyl glutamate has been replaced with a hydroxyl.

Significantly, the same phenomenon (a fraction of an acyl amino acid composition is 16 Dalton larger than expected) was observed regardless of whether the amino acid component of the acyl amino acid is glutamate or glycine. This observation indicates that a modification occurs on the fatty acid portion of an acyl amino acid, rather than on the amino acid portion.

Under certain growth conditions, the side-product with a greater mass can be as much as 99% of the total acyl amino acid composition. While the level of production of such a side-product can be reduced by controlling fermentation conditions, the present inventors have discovered use of strain engineering to reduce or completely eliminate production of the side-product. See, e.g., Example 2. In some circumstances where it is desirable to produce an acyl amino acid composition that is substantially free of acyl amino acids that have a hydroxyl group, e.g., at the ω-n position (where n≥1) of a fatty acid portion of the acyl amino acid, reducing or eliminating undesirable hydroxylation of a fatty acid portion of an acyl amino acid can be advantageous because modified molecules do not have the same physical properties as those of acyl amino acids without undesirable hydroxylation in some circumstances, and removing such modified molecules may reduce yield of desirable acyl amino acids. For example, in some embodiments, acid precipitation may be used as part of a purification protocol, and however, modified molecules do not precipitate efficiently, thereby reducing yield of desirable acyl amino acids.

### Example 2: Engineering of Microbial Cells to Comprise a Modification that Modulates Hydroxylation of a Fatty Acid Portion of an Acyl Amino Acid

The present Example describes construction and characterization of microbial cells that are engineered to produce acyl amino acids and to modulate hydroxylation of a fatty acid portion of the produced acyl amino acids. While this study assessed a reduction of hydroxylation of a fatty acid portion of an acyl amino acid when a gene associated with fatty acid hydroxylation is inactivated, those skilled in the art reading the present disclosure will also appreciate that hydroxylation of a fatty acid portion of an acyl amino acid can also be increased when a gene associated with fatty acid hydroxylation is activated or over-expressed. Further, those skilled in the art reading the present disclosure will also appreciate that technologies provided herein can be used to produce different acyl amino acids.

In this Example, microbial cells that produce acyl amino acids were engineered to inactivate a gene encoding a fatty acid hydroxylase (e.g., cypB). Accordingly, the present Example describes, for example, engineering a CypB-Upp-Kan construct. For example, genomic DNA from OKB105Δ(upp)Spect^{R}FA-GLU-TE-MG that encodes the cypB gene was amplified, for example, using primers:

55748:
(SEQ ID NO: 9)
5′-AAACTAAAAAAGGGTAGCCTAAAAA-3′
and
55749:
(SEQ ID NO: 10)
5′-AAAGAAGTTTTAGCTATAGGAGATTCC-3′.

The cypB gene and flanking sequence were amplified, for example, via nested PCR using primers:

55789:
(SEQ ID NO: 11)
5′-GATTGTACTGAGAGTGCACCATAGCTTGCATTAAGAGAAATTTAC
A-3′;
and
55790:
(SEQ ID NO: 12)
5′-GCGGTATTTCACACCGcAGGGAATACAAGTCTTTTAATCAG-3′

Such a fragment was annealed to a PCR product obtained from a template vector pUC19 amplified, for example, with primers

50348
(5′-GCGGTGTGAAATACCGcACAGATGCGTAAGGAGAAAA-3′ (SEQ
ID NO: 13))
and
50349
(5′-ATATGGTGCACTCTCAGTACAATcTGCTCTGATGCCGCA-3′
(SEQ ID NO: 14)).

The annealed mixture was transformed into cells (e.g., AbleK cells) to produce the plasmid cypB-bs168-inter-1-pUC19c, the nucleotide sequence of which is shown below:

| cypB-bs168-inter-1-pUC19c | | |
|---|---|---|
| (SEQ ID NO: 15) | | |
| 1 | TCGCGCGTTTCGGTGATGACGGTGAAAACCTCTGACACATGCAGCTCCCG | 50 |
| 51 | GAGACGGTCACAGCTTGTCTGTAAGCGGATGCCGGGAGCAGACAAGCCCG | 100 |
| 101 | TCAGGGCGCGTCAGCGGGTGTTGGCGGGTGTCGGGGCTGGCTTAACTATG | 150 |
| 151 | CGGCATCAGAGCAGATTGTACTGAGAGTGCACCATATGCTTGCATTAAGA | 200 |
| 201 | GAAATTTACAGGGTGCTTCAAATTGACGGCCGGTTTTATATCAGTATTGA | 250 |
| 251 | CACAAATACCGGTGAAAAAGAGAAAACGTATATTCAACTGCTGAAAGACC | 300 |
| 301 | AGCATTTCAGGGATCTTTCTGTTATCAGGCGTGCTTCCTGTCTATGTATT | 350 |
| 351 | GTGGCTGTTAAATAAAAAATTTCTCGGGAAATATATCCAAGATCCTCGTA | 400 |
| 401 | TTAGGATTTGGGTATATTTTCTTAATTTTTTATTTTTTGCATATACTTTA | 450 |
| 451 | TATTAAAAAAAGTTTTTTTCATATAAACTTATAACAGAAGAAAGAACAAA | 500 |
| 501 | GAGGTGATATCAGACAGGGCAGACATTCTTTGTGAACAAAAGGAATGAAT | 550 |
| 551 | ATTCATTCCGTAAACGAATCGGAGGTTGTCAGATTACATGATATCCGCAT | 600 |
| 601 | CCAGCAGTAAATACGACATGATTATGAAAGCGTCAGTCTCACTTTTTACG | 650 |
| 651 | GAAAGGGGTTTTGACGCTACCACTATTCCTATGATAGCTGAACGTGCTCA | 700 |
| 701 | TGTAGGGACAGGAACGATCTATCGTTATTTTGACAGCAAAGAAACACTCG | 750 |
| 751 | TTAACGTACTGTTTCAAGAAAGCATCCAGCGATTTACGGAAAAACTGAAG | 800 |
| 801 | CAAGACGTTTCAGAATTGCCTGTCAGAGAAGGCTTTCACCACGTATTTTG | 850 |
| 851 | CTGTCTCGTTCAGTTTACGAAAGAGAGCGACTATGCGCTTTTTTTTCTTG | 900 |
| 901 | AAACCAAAAAAGACGCTCATTACTTAAATCATACAAGCAAAAAAATGATA | 950 |
| 951 | GAAAATCTGACTCAAATGCTTGATGACTATTTTAATAAGGGAAAAGCGGA | 1000 |
| 1001 | AGGCGTGATTCGCAGCCTGCCCTCTAATGTGTTAATTGCGATTGTATTAG | 1050 |
| 1051 | GGGCGTTTCTCAAGATATATCAGCTCGTTCAAACAGGTGATATAGAGATG | 1100 |
| 1101 | GACACTGATTTAATTACTGAATTGGAACAATGCTGCTGGGACGCCATTAA | 1150 |
| 1151 | GCTTCATTCATCACAAAAATAGGAAAGGGAGATGTTAATGAAACAGGCAA | 200 |
| 1201 | GCGCAATACCTCAGCCCAAAACATACGGACCTTTAAAAAATCTTCCGCAT | 1250 |
| 1251 | CTGGAAAAAGAACAGCTTTCTCAATCCTTATGGCGGATAGCTGATGAATT | 1300 |
| 1301 | GGGACCGATTTTCCGTTTTGATTTTCCGGGAGTATCCAGTGTTTTTGTGT | 1350 |
| 1351 | CCGGCCACAATCTTGTGGCTGAAGTGTGTGATGAAAAACGCTTTGACAAG | 1400 |
| 1401 | AACCTTGGCAAAGGCTTGCAAAAGGTGCGTGAGTTCGGGGGAGATGGCTT | 1450 |
| 1451 | ATTTACAAGCTGGACGCACGAACCGAACTGGCAAAAAGCCCACCGCATTT | 1500 |
| 1501 | TGCTGCCGAGTTTTAGTCAAAAAGCGATGAAAGGCTATCATTCTATGATG | 1550 |
| 1551 | CTGGATATCGCAACCCAGCTGATTCAAAAGTGGAGCCGGTTAAACCCTAA | 1600 |
| 1601 | TGAAGAAATTGATGTAGCGGACGATATGACACGTCTGACGCTTGATACGA | 1650 |
| 1651 | TTGGGTTATGCGGGTTTAACTATCGATTCAACAGCTTTTACCGTGATTCA | 1700 |
| 1701 | CAGCATCCGTTTATCACCAGTATGCTCCGTGCCTTAAAAGAGGCGATGAA | 1750 |
| 1751 | TCAATCGAAAAGACTGGGCCTGCAAGATAAAATGATGGTGAAAACGAAGC | 1800 |
| 1801 | TGCAGTTCCAAAAGGATATAGAAGTCATGAACTCCCTGGTTGATAGAATG | 1850 |
| 1851 | ATAGCGGAGCGAAAGGCGAATCCGGATGAAAACATTAAGGATCTCTTGTC | 1900 |
| 1901 | TCTCATGCTTTATGCCAAAGATCCAGTAACGGGTGAAACGCTGGATGACG | 1950 |
| 1951 | AAAACATTCGATACCAAATCATCACATTTTTAATTGCTGGACATGAGACA | 2000 |
| 2001 | ACAAGCGGGTTGCTATCCTTTGCGATTTATTGTCTGCTTACACATCCGGA | 2050 |
| 2051 | AAAACTGAAAAAAGCTCAGGAGGAAGCGGATCGCGTGTTAACGGATGACA | 2100 |
| 2101 | CGCCTGAATATAAACAAATCCAGCAGCTCAAATACATTCGGATGGTTTTA | 2150 |
| 2151 | AATGAAACCCTCAGACTGTATCCAACAGCTCCGGCTTTTTCTCTATATGC | 2200 |
| 2201 | GAAGGAGGATACTGTTCTAGGCGGGGAATATCCGATCAGCAAAGGGCAGC | 2250 |
| 2251 | CAGTCACTGTTTTAATTCCAAAACTGCACCGGGATCAAAACGCTTGGGGA | 2300 |
| 2301 | CCGGATGCGGAAGATTTCCGTCCGGAACGGTTTGAGGATCCTTCAAGTAT | 2350 |
| 2351 | CCCTCACCATGCGTATAAGCCGTTTGGAAACGGACAGCGCGCTTGTATTG | 2400 |
| 2401 | GCATGCAGTTTGCTCTTCAAGAAGCGACAATGGTTCTCGGTCTTGTATTA | 2450 |
| 2451 | AAGCATTTTGAATTGATAAACCATACTGGCTACGAACTAAAAATCAAAGA | 2500 |
| 2501 | AGCATTAACGATCAAGCCGGATGATTTTAAAATTACTGTGAAACCGCGAA | 2550 |
| 2551 | AAACAGCGGCAATCAATGTACAGAGAAAAGAACAGGCAGACATCAAAGCA | 2600 |
| 2601 | GAAACAAAGCCAAAAGAAACCAAACCTAAACACGGCACACCTTTACTTGT | 2650 |
| 2651 | TCTTTTTGGTTCAAATCTTGGGACAGCTGAGGGAATAGCCGGTGAACTGG | 2700 |
| 2701 | CTGCTCAAGGCCGCCAGATGGGCTTTACAGCTGAAACGGCTCCGCTTGAT | 2750 |
| 2751 | GATTATATCGGCAAGCTCCCTGAAGAAGGGGCAGTCGTCATTGTAACGGC | 2800 |
| 2801 | TTCTTATAATGGGGCGCCGCCTGATAATGCTGCCGGATTTGTAGAGTGGC | 2850 |
| 2851 | TGAAAGAGCTTGAGGAAGGCCAATTGAAAGGTGTTTCCTATGCGGTATTC | 2900 |
| 2901 | GGCTGCGGAAACCGGAGCTGGGCCAGCACGTATCAGCGGATTCCCCGCCT | 2950 |
| 2951 | GATTGATGACATGATGAAAGCAAAGGGGGCATCGCGTTTAACAGCGATTG | 3000 |
| 3001 | GGGAAGGTGACGCCGCCGATGATTTTGAAAGCCACCGCGAGTCTTGGGAA | 3050 |
| 3051 | AACCGCTTCTGGAAGGAAACGATGGACGCATTTGATATTAACGAAATAGC | 3100 |
| 3101 | CCAGAAAGAAGACAGGCCTTCATTATCGATTACTTTTCTCAGTGAAGCGA | 3150 |
| 3151 | CGGAAACGCCGGTTGCTAAAGCATATGGCGCGTTTGAAGGGATTGTGTTA | 3200 |
| 3201 | GAGAATCGAGAACTCCAGACAGCTGCCAGCACGCGTTCAACCCGCCATAT | 3250 |
| 3251 | TGAATTGGAAATTCCGGCTGGTAAAACATATAAAGAAGGCGATCATATCG | 3300 |
| 3301 | GAATCCTGCCAAAGAACAGCAGGGAGCTTGTTCAGCGGGTTCTCAGCCGA | 3350 |
| 3351 | TTCGGTTTGCAGTCCAATCATGTGATAAAAGTAAGCGGAAGCGCTCATAT | 3400 |
| 3401 | GGCTCATCTGCCGATGGATCGGCCAATCAAAGTAGTGGATTTATTGTCGT | 3450 |
| 3451 | CCTATGTAGAGCTGCAGGAACCGGCATCAAGGCTTCAGCTTCGGGAGCTG | 3500 |
| 3501 | GCCTCTTATACAGTTTGTCCGCCGCATCAAAAAGAGCTGGAACAGCTCGT | 3550 |
| 3551 | TTCAGATGATGGCATTTACAAAGAGCAGGTACTTGCAAAACGTCTTACCA | 3600 |
| 3601 | TGCTTGATTTTTTAGAGGATTATCCTGCTTGCGAAATGCCGTTTGAACGG | 3650 |
| 3651 | TTTTTAGCACTTTTGCCATCACTAAAACCGAGATACTATTCCATTTCAAG | 3700 |
| 3701 | CTCACCGAAAGTTCATGCAAATATCGTGAGCATGACGGTAGGAGTTGTGA | 3750 |
| 3751 | AAGCCTCAGCATGGAGCGGCCGAGGTGAATACCGGGGTGTCGCCTCTAAT | 3800 |
| 3801 | TATTTAGCAGAATTGAATACAGGTGATGCAGCAGCTTGCTTCATTCGTAC | 3850 |
| 3851 | GCCGCAGTCCGGATTTCAGATGCCGAATGATCCTGAAACGCCTATGATTA | 3900 |
| 3901 | TGGTCGGGCCGGGCACAGGAATTGCGCCATTCAGAGGCTTTATTCAGGCA | 3950 |
| 3951 | AGATCGGTTTTGAAGAAGGAAGGAAGCACCCTTGGTGAAGCACTTTTATA | 4000 |
| 4001 | CTTCGGCTGCCGCCGCCCGGACCATGACGACCTTTACAGAGAAGAGCTGG | 4050 |
| 4051 | ATCAAGCGGAACAGGACGGTTTGGTCACAATCCGCCGATGCTACTCGCGC | 4100 |
| 4101 | GTCGAAAACGAACCAAAAGGATATGTCCAGCACTTGCTCAAGCAAGATAC | 4150 |
| 4151 | GCAGAAATTGATGACACTCATTGAAAAAGGGGCTCATATTTACGTATGCG | 4200 |
| 4201 | GTGATGGATCGCAAATGGCTCCTGATGTAGAGAGAACTTTGCGATTGGCA | 4250 |
| 4251 | TATGAAGCTGAAAAAGCAGCAAGTCAGGAAGAATCAGCTGTATGGCTGCA | 4300 |
| 4301 | AAAGCTGCAAGATCAAAGACGTTATGTGAAAGACGTTTGGACAGGAATGT | 4350 |
| 4351 | AAAATATAAAATCCCGCCAATCTGATTGGCGGGATTGCTTTGCATATGAG | 4400 |
| 4401 | AAAACCGGCACGATAATGAAATCTACTGAAAAGGATGTTATAGGGAGTAT | 4450 |
| 4451 | CGCGCCGGCCTTATTATTCATATCGGCATGCAGAGGCAAAAGTTTAGTTC | 4500 |
| 4501 | TTTTTACCTTGTTTTTAAAAATAAATAGTCTGAAAGTCTTGTTTTTGATT | 4550 |
| 4551 | TTCGACTCAGGCTTTTGGCATTTTGTTCTTTCTTATTCCTTAAGATCAGT | 4600 |
| 4601 | CATCTGTTGAAGATTGATGCTTGAATTGCTGTTCCACATGCTTACGGTAA | 4650 |
| 4651 | TGAAAGTCATGAATCAGCCGAATGGTCGGCCTGATTAACAGCAGCAAACT | 4700 |
| 4701 | TCCGATCGCAAACAGCCATATCCCTGCCGACATTAACCGGTCATAAAAAA | 4750 |
| 4751 | AGAAAAAACTTCCAACGAGAAACATAGCACCGATGATAAAATCGTTTACT | 4800 |
| 4801 | GTATAAAGAACCTTATATCGTTTTTTGAAAAAAAGCTCATATCGTTTCAA | 4850 |
| 4851 | CTCTTTTTGGATGTCATGTTCTTCATTTCCTTTCATTATCATCCCTCCAT | 4900 |
| 4901 | TCAATTTTGGCTTACCCTTACGTAAACGGCATGTAAACATAAGGGCTGCC | 4950 |
| 4951 | TTGCTGAAAGAAGACATAATCAACGATCAGAAACTAAAGTAAAAAAGTGA | 5000 |
| 5001 | TCTGTATAGGATCTCTTTTTTACGATTTCATCGCCTGAACAATAAGAGCA | 5050 |
| 5051 | GTCAAAGTTTTTGCCCCTTTAGGAACCAAGTGAACACCATCAGGGGTAAA | 5100 |
| 5101 | ATATTCCGGATGCTGAAGAGCTTCTGTATGCCAGTCAACTAACGTAACAT | 5150 |
| 5151 | TTTGATGTGCGTGGGCCTGTTGCTGCAAAGATTCATTTACCTTGCTTTCC | 5200 |
| 5201 | CATTGGCGGGGAACCCGTGTATTGACAAGATAAATATGAGCTTTTGAGAA | 5250 |
| 5251 | AGATTGAAGCAGTTGTTCGATTTGGCTGTTTGTAAAATAGCCATTGGTTC | 5300 |
| 5301 | CAAGCTCAATGATGACAGCCTTGTTCGGCTGATTAAAAGACTTGTATTCC | 5350 |
| 5351 | CTGCGGTGTGAAATACCGCACAGATGCGTAAGGAGAAAATACCGCATCAG | 5400 |
| 5401 | GCGCCATTCGCCATTCAGGCTGCGCAACTGTTGGGAAGGGCGATCGGTGC | 5450 |
| 5451 | GGGCCTCTTCGCTATTACGCCAGCTGGCGAAAGGGGGATGTGCTGCAAGG | 5500 |
| 5501 | CGATTAAGTTGGGTAACGCCAGGGTTTTCCCAGTCACGACGTTGTAAAAC | 5550 |
| 5551 | GACGGCCAGTGAATTCGAGCTCGGTACCCGGGGATCCTCTAGAGTCGACC | 5600 |
| 5601 | TGCAGGCATGCAAGCTTGGCGTAATCATGGTCATAGCTGTTTCCTGTGTG | 5650 |
| 5651 | AAATTGTTATCCGCTCACAATTCCACACAACATACGAGCCGGAAGCATAA | 5700 |
| 5701 | AGTGTAAAGCCTGGGGTGCCTAATGAGTGAGCTAACTCACATTAATTGCG | 5750 |
| 5751 | TTGCGCTCACTGCCCGCTTTCCAGTCGGGAAACCTGTCGTGCCAGCTGCA | 5800 |
| 5801 | TTAATGAATCGGCCAACGCGCGGGGAGAGGCGGTTTGCGTATTGGGCGCT | 5850 |
| 5851 | CTTCCGCTTCCTCGCTCACTGACTCGCTGCGCTCGGTCGTTCGGCTGCGG | 5900 |
| 5901 | CGAGCGGTATCAGCTCACTCAAAGGCGGTAATACGGTTATCCACAGAATC | 5950 |
| 5951 | AGGGGATAACGCAGGAAAGAACATGTGAGCAAAAGGCCAGCAAAAGGCCA | 6000 |
| 6001 | GGAACCGTAAAAAGGCCGCGTTGCTGGCGTTTTTCCATAGGCTCCGCCCC | 6050 |
| 6051 | CCTGACGAGCATCACAAAAATCGACGCTCAAGTCAGAGGTGGCGAAACCC | 6100 |
| 6101 | GACAGGACTATAAAGATACCAGGCGTTTCCCCCTGGAAGCTCCCTCGTGC | 6150 |
| 6151 | GCTCTCCTGTTCCGACCCTGCCGCTTACCGGATACCTGTCCGCCTTTCTC | 6200 |
| 6201 | CCTTCGGGAAGCGTGGCGCTTTCTCATAGCTCACGCTGTAGGTATCTCAG | 6250 |
| 6251 | TTCGGTGTAGGTCGTTCGCTCCAAGCTGGGCTGTGTGCACGAACCCCCCG | 6300 |
| 6301 | TTCAGCCCGACCGCTGCGCCTTATCCGGTAACTATCGTCTTGAGTCCAAC | 6350 |
| 6351 | CCGGTAAGACACGACTTATCGCCACTGGCAGCAGCCACTGGTAACAGGAT | 6400 |
| 6401 | TAGCAGAGCGAGGTATGTAGGCGGTGCTACAGAGTTCTTGAAGTGGTGGC | 6450 |
| 6451 | CTAACTACGGCTACACTAGAAGAACAGTATTTGGTATCTGCGCTCTGCTG | 6500 |
| 6501 | AAGCCAGTTACCTTCGGAAAAAGAGTTGGTAGCTCTTGATCCGGCAAACA | 6550 |
| 6551 | AACCACCGCTGGTAGCGGTGGTTTTTTTGTTTGCAAGCAGCAGATTACGC | 6600 |
| 6601 | GCAGAAAAAAAGGATCTCAAGAAGATCCTTTGATCTTTTCTACGGGGTCT | 6650 |
| 6651 | GACGCTCAGTGGAACGAAAACTCACGTTAAGGGATTTTGGTCATGAGATT | 6700 |
| 6701 | ATCAAAAAGGATCTTCACCTAGATCCTTTTAAATTAAAAATGAAGTTTTA | 6750 |
| 6751 | AATCAATCTAAAGTATATATGAGTAAACTTGGTCTGACAGTTACCAATGC | 6800 |
| 6801 | TTAATCAGTGAGGCACCTATCTCAGCGATCTGTCTATTTCGTTCATCCAT | 6850 |
| 6851 | AGTTGCCTGACTCCCCGTCGTGTAGATAACTACGATACGGGAGGGCTTAC | 6900 |
| 6901 | CATCTGGCCCCAGTGCTGCAATGATACCGCGAGACCCACGCTCACCGGCT | 6950 |
| 6951 | CCAGATTTATCAGCAATAAACCAGCCAGCCGGAAGGGCCGAGCGCAGAAG | 7000 |
| 7001 | TGGTCCTGCAACTTTATCCGCCTCCATCCAGTCTATTAATTGTTGCCGGG | 7050 |
| 7051 | AAGCTAGAGTAAGTAGTTCGCCAGTTAATAGTTTGCGCAACGTTGTTGCC | 7100 |
| 7101 | ATTGCTACAGGCATCGTGGTGTCACGCTCGTCGTTTGGTATGGCTTCATT | 7150 |
| 7151 | CAGCTCCGGTTCCCAACGATCAAGGCGAGTTACATGATCCCCCATGTTGT | 7200 |
| 7201 | GCAAAAAAGCGGTTAGCTCCTTCGGTCCTCCGATCGTTGTCAGAAGTAAG | 7250 |
| 7251 | TTGGCCGCAGTGTTATCACTCATGGTTATGGCAGCACTGCATAATTCTCT | 7300 |
| 7301 | TACTGTCATGCCATCCGTAAGATGCTTTTCTGTGACTGGTGAGTACTCAA | 7350 |
| 7351 | CCAAGTCATTCTGAGAATAGTGTATGCGGCGACCGAGTTGCTCTTGCCCG | 7400 |
| 7401 | GCGTCAATACGGGATAATACCGCGCCACATAGCAGAACTTTAAAAGTGCT | 7450 |
| 7451 | CATCATTGGAAAACGTTCTTCGGGGCGAAAACTCTCAAGGATCTTACCGC | 7500 |
| 7501 | TGTTGAGATCCAGTTCGATGTAACCCACTCGTGCACCCAACTGATCTTCA | 7550 |
| 7551 | GCATCTTTTACTTTCACCAGCGTTTCTGGGTGAGCAAAAACAGGAAGGCA | 7600 |
| 7601 | AAATGCCGCAAAAAAGGGAATAAGGGCGACACGGAAATGTTGAATACTCA | 7650 |
| 7651 | TACTCTTCCTTTTTCAATATTATTGAAGCATTTATCAGGGTTATTGTCTC | 7700 |
| 7701 | ATGAGCGGATACATATTTGAATGTATTTAGAAAAATAAACAAATAGGGGT | 7750 |
| 7751 | TCCGCGCACATTTCCCCGAAAAGTGCCACCTGACGTCTAAGAAACCATTA | 7800 |
| 7801 | TTATCATGACATTAACCTATAAAAATAGGCGTATCACGAGGCCCTTTCGT | 7850 |
| 7851 | C | 7851 |

The plasmid cypB-bs168-inter-1-pUC19c was subsequently amplified with primers 55791 (5′-CGCAACTGTCCATACTCTgAATATAAAATCCCGCCAATCT-3′(SEQ ID NO: 16)) and 55792 (5′-GAACATCGTCAAAAAACCcTAACATCTCCCTTTCCTATTTTT-3′(SEQ ID NO: 17)) to produce a cypB knockout vector. The cypB knockout vector was annealed to a PCR product obtained from amplifying upp-kan from plasmid upp-kan-pUC19, for example, using primers 50374 (5′-GGGTTTTTTGACGATGTTcTTGAAACTCAATGTCTTTTTTT-3′(SEQ ID NO: 18)) and 50371 (5′-CAGAGTATGGACAGTTGCgGATGTACTTCAGAAAAGATTAGATG-3′(SEQ ID NO: 19)). The annealed mixture was transformed into cells (e.g., AbleK cells) to produce the plasmid cypB-bs168-precursor-pUC19c, the nucleotide sequence of which is shown below:

| cypB-bs168-precursor-pUC19c | | |
|---|---|---|
| (SEQ ID NO: 20) | | |
| 1 | TCGCGCGTTTCGGTGATGACGGTGAAAACCTCTGACACATGCAGCTCCCG | 50 |
| 51 | GAGACGGTCACAGCTTGTCTGTAAGCGGATGCCGGGAGCAGACAAGCCCG | 100 |
| 101 | TCAGGGCGCGTCAGCGGGTGTTGGCGGGTGTCGGGGCTGGCTTAACTATG | 150 |
| 151 | CGGCATCAGAGCAGATTGTACTGAGAGTGCACCATATGCTTGCATTAAGA | 200 |
| 201 | GAAATTTACAGGGTGCTTCAAATTGACGGCCGGTTTTATATCAGTATTGA | 250 |
| 251 | CACAAATACCGGTGAAAAAGAGAAAACGTATATTCAACTGCTGAAAGACC | 300 |
| 301 | AGCATTTCAGGGATCTTTCTGTTATCAGGCGTGCTTCCTGTCTATGTATT | 350 |
| 351 | GTGGCTGTTAAATAAAAAATTTCTCGGGAAATATATCCAAGATCCTCGTA | 400 |
| 401 | TTAGGATTTGGGTATATTTTCTTAATTTTTTATTTTTTGCATATACTTTA | 450 |
| 451 | TATTAAAAAAAGTTTTTTTCATATAAACTTATAACAGAAGAAAGAACAAA | 500 |
| 501 | GAGGTGATATCAGACAGGGCAGACATTCTTTGTGAACAAAAGGAATGAAT | 550 |
| 551 | ATTCATTCCGTAAACGAATCGGAGGTTGTCAGATTACATGATATCCGCAT | 600 |
| 601 | CCAGCAGTAAATACGACATGATTATGAAAGCGTCAGTCTCACTTTTTACG | 650 |
| 651 | GAAAGGGGTTTTGACGCTACCACTATTCCTATGATAGCTGAACGTGCTCA | 700 |
| 701 | TGTAGGGACAGGAACGATCTATCGTTATTTTGACAGCAAAGAAACACTCG | 750 |
| 751 | TTAACGTACTGTTTCAAGAAAGCATCCAGCGATTTACGGAAAAACTGAAG | 800 |
| 801 | CAAGACGTTTCAGAATTGCCTGTCAGAGAAGGCTTTCACCACGTATTTTG | 850 |
| 851 | CTGTCTCGTTCAGTTTACGAAAGAGAGCGACTATGCGCTTTTTTTTCTTG | 900 |
| 901 | AAACCAAAAAAGACGCTCATTACTTAAATCATACAAGCAAAAAAATGATA | 950 |
| 951 | GAAAATCTGACTCAAATGCTTGATGACTATTTTAATAAGGGAAAAGCGGA | 1000 |
| 1001 | AGGCGTGATTCGCAGCCTGCCCTCTAATGTGTTAATTGCGATTGTATTAG | 1050 |
| 1051 | GGGCGTTTCTCAAGATATATCAGCTCGTTCAAACAGGTGATATAGAGATG | 1100 |
| 1101 | GACACTGATTTAATTACTGAATTGGAACAATGCTGCTGGGACGCCATTAA | 1150 |
| 1151 | GCTTCATTCATCACAAAAATAGGAAAGGGAGATGTTAGGGTTTTTTGACG | 1200 |
| 1201 | ATGTTCTTGAAACTCAATGTCTTTTTTTGTAGAATCAATAGAAGTGTGTA | 1250 |
| 1251 | ATTGTTGATGGGACAATAAAAAAGGAGCTGAAACACAGTATGGGAAAGGT | 1300 |
| 1301 | TTATGTATTTGATCATCCTTTAATTCAGCACAAGCTGACATATATACGGA | 1350 |
| 1351 | ATGAAAATACAGGTACGAAGGATTTTAGAGAGTTAGTAGATGAAGTGGCT | 1400 |
| 1401 | ACACTCATGGCATTTGAAATTACCCGCGATCTTCCTCTGGAAGAAGTGGA | 1450 |
| 1451 | TATCAATACACCGGTTCAGGCTGCGAAATCGAAAGTCATCTCAGGGAAAA | 1500 |
| 1501 | AACTCGGAGTGGTTCCTATCCTCAGAGCAGGATTGGGAATGGTTGACGGC | 1550 |
| 1551 | ATTTTAAAGCTGATTCCTGCGGCAAAAGTGGGACATGTCGGCCTTTACCG | 1600 |
| 1601 | TGATCCAGAAACCTTAAAACCCGTGGAATACTATGTCAAGCTTCCTTCTG | 1650 |
| 1651 | ATGTGGAAGAGCGTGAATTCATCGTGGTTGACCCGATGCTCGCTACAGGC | 1700 |
| 1701 | GGTTCCGCAGTTGAAGCCATTCACAGCCTTAAAAAACGCGGTGCGAAAAA | 1750 |
| 1751 | TATCCGTTTCATGTGTCTTGTAGCAGCGCCGGAGGGTGTGGAAGAATTGC | 1800 |
| 1801 | AGAAGCATCATTCGGACGTTGATATTTACATTGCGGCGCTAGATGAAAAA | 1850 |
| 1851 | TTAAATGAAAAAGGATATATTGTTCCAGGTCTCGGAGATGCGGGTGACCG | 1900 |
| 1901 | CATGTTTGGAACAAAATAAAAAATGAAATCCCCAAAAGGGGGTTTCATTT | 1950 |
| 1951 | TTTTATCCAGTTTTTTGCTATTCGGTGAATCTGTATACAATTATAGGTGA | 2000 |
| 2001 | AAATGTGAACATTCTGGGATCCGATAAACCCAGCGAACCATTTGAGGTGA | 2050 |
| 2051 | TAGGTAAGATTATACCGAGGTATGAAAACGAGAATTGGACCTTTACAGAA | 2100 |
| 2101 | TTACTCTATGAAGCGCCATATTTAAAAAGCTACCAAGACGAAGAGGATGA | 2150 |
| 2151 | AGAGGATGAGGAGGCAGATTGCCTTGAATATATTGACAATACTGATAAGA | 2200 |
| 2201 | TAATATATCTTTTATATAGAAGATATCGCCGTATGTAAGGATTTCAGGGG | 2250 |
| 2251 | GCAAGGCATAGGCAGCGCGCTTATCAATATATCTATAGAATGGGCAAAGC | 2300 |
| 2301 | ATAAAAACTTGCATGGACTAATGCTTGAAACCCAGGACAATAACCTTATA | 2350 |
| 2351 | GCTTGTAAATTCTATCATAATTGTGGTTTCAAAATCGGCTCCGTCGATAC | 2400 |
| 2401 | TATGTTATACGCCAACTTTCAAAACAACTTTGAAAAAGCTGTTTTCTGGT | 2450 |
| 2451 | ATTTAAGGTTTTAGAATGCAAGGAACAGTGAATTGGAGTTCGTCTTGTTA | 2500 |
| 2501 | TAATTAGCTTCTTGGGGTATCTTTAAATACTGTAGAAAAGAGGAAGGAAA | 2550 |
| 2551 | TAATAAATGGCTAAAATGAGAATATCACCGGAATTGAAAAAACTGATCGA | 2600 |
| 2601 | AAAATACCGCTGCGTAAAAGATACGGAAGGAATGTCTCCTGCTAAGGTAT | 2650 |
| 2651 | ATAAGCTGGTGGGAGAAAATGAAAACCTATATTTAAAAATGACGGACAGC | 2700 |
| 2701 | CGGTATAAAGGGACCACCTATGATGTGGAACGGGAAAAGGACATGATGCT | 2750 |
| 2751 | ATGGCTGGAAGGAAAGCTGCCTGTTCCAAAGGTCCTGCACTTTGAACGGC | 2800 |
| 2801 | ATGATGGCTGGAGCAATCTGCTCATGAGTGAGGCCGATGGCGTCCTTTGC | 2850 |
| 2851 | TCGGAAGAGTATGAAGATGAACAAAGCCCTGAAAAGATTATCGAGCTGTA | 2900 |
| 2901 | TGCGGAGTGCATCAGGCTCTTTCACTCCATCGACATATCGGATTGTCCCT | 2950 |
| 2951 | ATACGAATAGCTTAGACAGCCGCTTAGCCGAATTGGATTACTTACTGAAT | 3000 |
| 3001 | AACGATCTGGCCGATGTGGATTGCGAAAACTGGGAAGAAGACACTCCATT | 3050 |
| 3051 | TAAAGATCCGCGCGAGCTGTATGATTTTTTAAAGACGGAAAAGCCCGAAG | 3100 |
| 3101 | AGGAACTTGTCTTTTCCCACGGCGACCTGGGAGACAGCAACATCTTTGTG | 3150 |
| 3151 | AAAGATGGCAAAGTAAGTGGCTTTATTGATCTTGGGAGAAGCGGCAGGGC | 3200 |
| 3201 | GGACAAGTGGTATGACATTGCCTTCTGCGTCCGGTCGATCAGGGAGGATA | 3250 |
| 3251 | TCGGGGAAGAACAGTATGTCGAGCTATTTTTTGACTTACTGGGGATCAAG | 3300 |
| 3301 | CCTGATTGGGAGAAAATAAAATATTATATTTTACTGGATGAATTGTTTTA | 3350 |
| 3351 | GTACCTAGATTTAGATGTCTAAAAAGCTTTAACTACAAGCTTTTTAGACA | 3400 |
| 3401 | TCTAATCTTTTCTGAAGTACATCCGCAACTGTCCATACTCTGAATATAAA | 3450 |
| 3451 | ATCCCGCCAATCTGATTGGCGGGATTGCTTTGCATATGAGAAAACCGGCA | 3500 |
| 3501 | CGATAATGAAATCTACTGAAAAGGATGTTATAGGGAGTATCGCGCCGGCC | 3550 |
| 3551 | TTATTATTCATATCGGCATGCAGAGGCAAAAGTTTAGTTCTTTTTACCTT | 3600 |
| 3601 | GTTTTTAAAAATAAATAGTCTGAAAGTCTTGTTTTTGATTTTCGACTCAG | 3650 |
| 3651 | GCTTTTGGCATTTTGTTCTTTCTTATTCCTTAAGATCAGTCATCTGTTGA | 3700 |
| 3701 | AGATTGATGCTTGAATTGCTGTTCCACATGCTTACGGTAATGAAAGTCAT | 3750 |
| 3751 | GAATCAGCCGAATGGTCGGCCTGATTAACAGCAGCAAACTTCCGATCGCA | 3800 |
| 3801 | AACAGCCATATCCCTGCCGACATTAACCGGTCATAAAAAAAGAAAAAACT | 3850 |
| 3851 | TCCAACGAGAAACATAGCACCGATGATAAAATCGTTTACTGTATAAAGAA | 3900 |
| 3901 | CCTTATATCGTTTTTTGAAAAAAAGCTCATATCGTTTCAACTCTTTTTGG | 3950 |
| 3951 | ATGTCATGTTCTTCATTTCCTTTCATTATCATCCCTCCATTCAATTTTGG | 4000 |
| 4001 | CTTACCCTTACGTAAACGGCATGTAAACATAAGGGCTGCCTTGCTGAAAG | 4050 |
| 4051 | AAGACATAATCAACGATCAGAAACTAAAGTAAAAAAGTGATCTGTATAGG | 4100 |
| 4101 | ATCTCTTTTTTACGATTTCATCGCCTGAACAATAAGAGCAGTCAAAGTTT | 4150 |
| 4151 | TTGCCCCTTTAGGAACCAAGTGAACACCATCAGGGGTAAAATATTCCGGA | 4200 |
| 4201 | TGCTGAAGAGCTTCTGTATGCCAGTCAACTAACGTAACATTTTGATGTGC | 4250 |
| 4251 | GTGGGCCTGTTGCTGCAAAGATTCATTTACCTTGCTTTCCCATTGGCGGG | 4300 |
| 4301 | GAACCCGTGTATTGACAAGATAAATATGAGCTTTTGAGAAAGATTGAAGC | 4350 |
| 4351 | AGTTGTTCGATTTGGCTGTTTGTAAAATAGCCATTGGTTCCAAGCTCAAT | 4400 |
| 4401 | GATGACAGCCTTGTTCGGCTGATTAAAAGACTTGTATTCCCTGCGGTGTG | 4450 |
| 4451 | AAATACCGCACAGATGCGTAAGGAGAAAATACCGCATCAGGCGCCATTCG | 4500 |
| 4501 | CCATTCAGGCTGCGCAACTGTTGGGAAGGGCGATCGGTGCGGGCCTCTTC | 4550 |
| 4551 | GCTATTACGCCAGCTGGCGAAAGGGGGATGTGCTGCAAGGCGATTAAGTT | 4600 |
| 4601 | GGGTAACGCCAGGGTTTTCCCAGTCACGACGTTGTAAAACGACGGCCAGT | 4650 |
| 4651 | GAATTCGAGCTCGGTACCCGGGGATCCTCTAGAGTCGACCTGCAGGCATG | 4700 |
| 4701 | CAAGCTTGGCGTAATCATGGTCATAGCTGTTTCCTGTGTGAAATTGTTAT | 4750 |
| 4751 | CCGCTCACAATTCCACACAACATACGAGCCGGAAGCATAAAGTGTAAAGC | 4800 |
| 4801 | CTGGGGTGCCTAATGAGTGAGCTAACTCACATTAATTGCGTTGCGCTCAC | 4850 |
| 4851 | TGCCCGCTTTCCAGTCGGGAAACCTGTCGTGCCAGCTGCATTAATGAATC | 4900 |
| 4901 | GGCCAACGCGCGGGGAGAGGCGGTTTGCGTATTGGGCGCTCTTCCGCTTC | 4950 |
| 4951 | CTCGCTCACTGACTCGCTGCGCTCGGTCGTTCGGCTGCGGCGAGCGGTAT | 5000 |
| 5001 | CAGCTCACTCAAAGGCGGTAATACGGTTATCCACAGAATCAGGGGATAAC | 5050 |
| 5051 | GCAGGAAAGAACATGTGAGCAAAAGGCCAGCAAAAGGCCAGGAACCGTAA | 5100 |
| 5101 | AAAGGCCGCGTTGCTGGCGTTTTTCCATAGGCTCCGCCCCCCTGACGAGC | 5150 |
| 5151 | ATCACAAAAATCGACGCTCAAGTCAGAGGTGGCGAAACCCGACAGGACTA | 5200 |
| 5201 | TAAAGATACCAGGCGTTTCCCCCTGGAAGCTCCCTCGTGCGCTCTCCTGT | 5250 |
| 5251 | TCCGACCCTGCCGCTTACCGGATACCTGTCCGCCTTTCTCCCTTCGGGAA | 5300 |
| 5301 | GCGTGGCGCTTTCTCATAGCTCACGCTGTAGGTATCTCAGTTCGGTGTAG | 5350 |
| 5351 | GTCGTTCGCTCCAAGCTGGGCTGTGTGCACGAACCCCCCGTTCAGCCCGA | 5400 |
| 5401 | CCGCTGCGCCTTATCCGGTAACTATCGTCTTGAGTCCAACCCGGTAAGAC | 5450 |
| 5451 | ACGACTTATCGCCACTGGCAGCAGCCACTGGTAACAGGATTAGCAGAGCG | 5500 |
| 5501 | AGGTATGTAGGCGGTGCTACAGAGTTCTTGAAGTGGTGGCCTAACTACGG | 5550 |
| 5551 | CTACACTAGAAGAACAGTATTTGGTATCTGCGCTCTGCTGAAGCCAGTTA | 5600 |
| 5601 | CCTTCGGAAAAAGAGTTGGTAGCTCTTGATCCGGCAAACAAACCACCGCT | 5650 |
| 5651 | GGTAGCGGTGGTTTTTTTGTTTGCAAGCAGCAGATTACGCGCAGAAAAAA | 5700 |
| 5701 | AGGATCTCAAGAAGATCCTTTGATCTTTTCTACGGGGTCTGACGCTCAGT | 5750 |
| 5751 | GGAACGAAAACTCACGTTAAGGGATTTTGGTCATGAGATTATCAAAAAGG | 5800 |
| 5801 | ATCTTCACCTAGATCCTTTTAAATTAAAAATGAAGTTTTAAATCAATCTA | 5850 |
| 5851 | AAGTATATATGAGTAAACTTGGTCTGACAGTTACCAATGCTTAATCAGTG | 5900 |
| 5901 | AGGCACCTATCTCAGCGATCTGTCTATTTCGTTCATCCATAGTTGCCTGA | 5950 |
| 5951 | CTCCCCGTCGTGTAGATAACTACGATACGGGAGGGCTTACCATCTGGCCC | 6000 |
| 6001 | CAGTGCTGCAATGATACCGCGAGACCCACGCTCACCGGCTCCAGATTTAT | 6050 |
| 6051 | CAGCAATAAACCAGCCAGCCGGAAGGGCCGAGCGCAGAAGTGGTCCTGCA | 6100 |
| 6101 | ACTTTATCCGCCTCCATCCAGTCTATTAATTGTTGCCGGGAAGCTAGAGT | 6150 |
| 6151 | AAGTAGTTCGCCAGTTAATAGTTTGCGCAACGTTGTTGCCATTGCTACAG | 6200 |
| 6201 | GCATCGTGGTGTCACGCTCGTCGTTTGGTATGGCTTCATTCAGCTCCGGT | 6250 |
| 6251 | TCCCAACGATCAAGGCGAGTTACATGATCCCCCATGTTGTGCAAAAAAGC | 6300 |
| 6301 | GGTTAGCTCCTTCGGTCCTCCGATCGTTGTCAGAAGTAAGTTGGCCGCAG | 6350 |
| 6351 | TGTTATCACTCATGGTTATGGCAGCACTGCATAATTCTCTTACTGTCATG | 6400 |
| 6401 | CCATCCGTAAGATGCTTTTCTGTGACTGGTGAGTACTCAACCAAGTCATT | 6450 |
| 6451 | CTGAGAATAGTGTATGCGGCGACCGAGTTGCTCTTGCCCGGCGTCAATAC | 6500 |
| 6501 | GGGATAATACCGCGCCACATAGCAGAACTTTAAAAGTGCTCATCATTGGA | 6550 |
| 6551 | AAACGTTCTTCGGGGCGAAAACTCTCAAGGATCTTACCGCTGTTGAGATC | 6600 |
| 6601 | CAGTTCGATGTAACCCACTCGTGCACCCAACTGATCTTCAGCATCTTTTA | 6650 |
| 6651 | CTTTCACCAGCGTTTCTGGGTGAGCAAAAACAGGAAGGCAAAATGCCGCA | 6700 |
| 6701 | AAAAAGGGAATAAGGGCGACACGGAAATGTTGAATACTCATACTCTTCCT | 6750 |
| 6751 | TTTTCAATATTATTGAAGCATTTATCAGGGTTATTGTCTCATGAGCGGAT | 6800 |
| 6801 | ACATATTTGAATGTATTTAGAAAAATAAACAAATAGGGGTTCCGCGCACA | 6850 |
| 6851 | TTTCCCCGAAAAGTGCCACCTGACGTCTAAGAAACCATTATTATCATGAC | 6900 |
| 6901 | ATTAACCTATAAAAATAGGCGTATCACGAGGCCCTTTCGTC | 6941 |

The plasmid cypB-bs168-precursor-pUC19c was used to transform 59196-A8 OKB105-sfp+, phe+, upp::spc, amyE::Psrf-comS-PgroEL-sfp-srfD, srf::psrf-FA-Glu, maf:psrf-FA-Glu, eps::psrf-FA-Glu, rapC::psrf-FA-Glu, yngF::psrf-FA-Glu, pps::CAM, lacA:xylR-pxylA-comK-ermR, spoIIAC:ko, degU::ko. A resulting strain was named “strain 61000-A5.”

Cultures of strain 61000-A5 were inoculated (e.g., from glycerol scraping) into cell culture media (e.g., 10 mL S7 (Phos7.5) media) and grown in shaking incubator for an appropriate period of time (e.g., 4 days). An aliquot of culture (e.g., 1 mL of culture) was then sampled for characterization and analysis, for example, by liquid chromatography-mass spectrometry (LC-MS). For example, an aliquot of culture was centrifuged at 13,500 RPM for 5 minutes and supernatant was filtered through 0.45 μM Durapore® membrane columns at 7000 g for 1 minute and filtrate was diluted 1:200 for LC-MS analysis.

Results and Discussion: Analysis of production of FA-Glu by strain 61000-A5 shows that strain 61000-A5 was able to produce FA-Glu with no additional hydroxyl groups. Such data was obtained using LC-MS analysis. In some embodiments, an acyl amino acid composition comprising substantially 100% FA-Glu with substantially no additional hydroxyl groups (except that in some embodiments, FA-Glu may comprise a β-hydroxyl group). This Example shows that inactivation of a gene encoding a fatty acid hydroxylase (e.g., cypB gene) reduced the level of modification (hydroxylation) of acyl amino acids, e.g., FA-Gly and FA-Glu, to very low—nearly undetectable—levels. Therefore, one aspect of the present disclosure encompasses the insight that acyl amino acid-producing cells with a modification that modulates hydroxylation of a fatty acid portion of an acyl amino acid (e.g., by inactivating one or more genes encoding a fatty acid hydroxylase such as, e.g., cypB gene) can be useful to production of surfactants and fatty acids for commercial purposes. The present disclosure provides, among others, insights that fatty acid hydroxylation by a cypB enzyme expressed by acyl amino acid-producing cells can be undesirable in certain circumstances where desirable surfactants and/or fatty acid do not comprise additional hydroxyl groups (except that in some embodiments, such desirable surfactants and/or fatty acid may have a β-hydroxyl), and that the presence of such hydroxylated products may lower the yield of the desired surfactants and/or fatty acid. The present inventors demonstrated that inactivation of a fatty acid hydroxylase (e.g., by inactivating a gene encoding cypB) in acyl amino acid-producing cells increased yield of the desired surfactant and/or fatty acids. Accordingly, one aspect of the present disclosure provides technologies for increasing yield of surfactants and/or fatty acids of interest (e.g., without ω-n hydroxyl groups, where n≥1).

### Example 3: Production of Acyl Amino Acids Using a Microbial Catalyst (with or without a Modification that Modulates Hydroxylation of a Fatty Acid Portion of Acyl Amino Acids)

Surfactants such as acyl amino acids, e.g., capryloyl glycine, lauroyl glycinate, myristoyl glycinate, and cocoyl glycinate, are typically manufactured using hazardous chemical processes, such as chlorination of fatty acids (as described in U.S. Pat. No. 8,338,483). Disclosed herein are methods for producing acyl amino acid-based surfactants using a microbial catalysis. For example, in some embodiments, microbial cells (e.g., Bacillus subtilis) are cultured or grown in a culture medium containing a carbon source, such as, e.g., glucose, a fatty acid (which is selected to form a fatty acid portion of an acyl amino acid), and an amino acid (which is selected to form an amino acid portion of an acyl amino acid) in the presence of an enzyme that covalently links a fatty acid to an amino acid under conditions and for a time sufficient for an acyl amino acid position to be made. In some embodiments, glycine may be selected as an amino acid provided in a culture medium. Substrates are converted by microbial cells (e.g., Bacillus cells) into desired acyl amino acid surfactants depending on the selection of fatty acids and amino acid. By way of example only, lauroyl glycinate is produced when lauric acid and glycine are provided in a culture medium, in which microbial cells are culture or grown in the presence of an enzyme that covalently links a fatty acid to an amino acid under conditions and for a time sufficient for an acyl amino acid position to be made. In some embodiments, such microbial cells may be engineered to comprise modification that modulates hydroxylation of a fatty acid portion of an acyl amino acid (e.g., as described in Example 2).

In some embodiments, strains were inoculated (e.g., from glycerol scraping) into 10 mL cultures containing LB+Ery (LB media with erythromycin). Cultures were grown for ˜20 hrs at 37° C. with 200 RPM agitation. 100 μL of this seed culture was then used to inoculate flasks containing 50 mL S7(Phos7.5)Gly+Ery. Cultures were grown as described herein for ˜24 hrs before being split into 4×10 mL cultures. These split cultures were each supplemented with no fatty acid or a fatty acid as indicated: (a) no fatty acid, (b) octanoic acid (e.g., 5 μL 200 mM; diluted from 98% stock Sigma W279900), (c) lauric acid (e.g., 5 μL 200 mM; Sigma W261408 resuspended in 200 proof ethanol), or (d) myristic acid (e.g., 5 μL 200 mM; Sigma 70082 resuspended in 200 proof ethanol). Supplemented cultures were grown as above for ˜6 hrs before a 0.5 mL sample removed and frozen at ˜20° C. Cultures were returned to an incubator. Cultures were sampled again as described above at ˜24 hrs post-supplementation. Frozen samples were thawed and centrifuged at 13,500 RPM×5 min. 250 uL supernatant was filtered through 0.45 uM filter plate at 5000 g×10 min. Filtrate was diluted 1:20 for LCMS analysis.

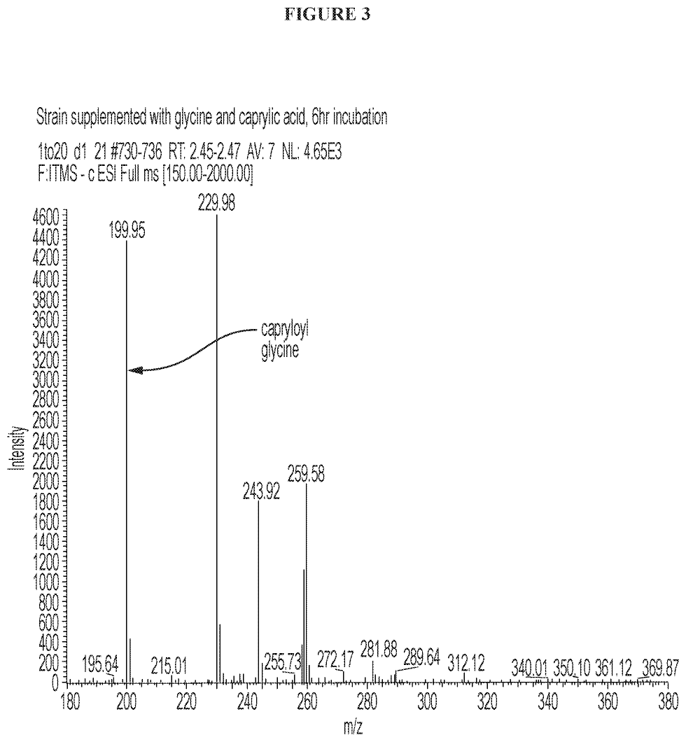

Octanoic acid: Analysis of cultures supplemented with octanoic acid showed a unique peak at ˜200 Da corresponding to C8-Glycine (FIG. 3). This peak eluted at about the same retention time as capryloyl glycine (Carbosynth FC15595). As discussed herein, the present disclosure recognizes that surfactants produced in Bacillus are often modified by hydroxylation. A peak corresponding to a higher molecular weight synthesis product with a mass of about 230 Dalton was also detected. It was contemplated that such a higher molecular weight synthesis product may be resulted from both hydroxylation and methylation of C8-glycine. See, e.g., Sun et al. Sci Total Environ (2018) 613-614: 54-61. Both peak signals were strongest in cultures collected at T=6 hr; and were weaker at 24 hours post-addition of substrates. Significantly, both peak signals remained strong, even after 24 hours of incubation in a strain in which a fatty acid hydroxylase is inactivated (e.g., as described in Example 1), e.g., by knocking out cypB gene, which indicates that cypB plays a role in hydroxylation of the C8-glycine, converting it into higher molecule weight modified (hydroxylated) forms. In some embodiments, production of acyl amino acid with one or more ω-n hydroxyl groups (where n≥1) can be reduced or eliminated by deletion of one or more genes encoding a fatty acid hydroxylase, such as cypB. Other peak signals were observed with masses of 244, 259, and 272 Dalton.

Lauric acid: Analysis of cultures supplemented with lauric acid showed a small unique peak at ˜272 Dalton, which is contemplated to be corresponding to C12-Glycine-OH. An additional unique peak at ˜302 Da, which is contemplated to be corresponding to C12-Me-2(OH)-glycine, was stable over time. Other peak signals correspond to species with a mass of 316 Dalton.

Myristic acid: Analysis of the cultures supplemented with myristic acid supplemented showed a unique peak at ˜330 Dalton, which is contemplated to be corresponding to C14-2(Me)-OH-glycine, was stable over time. Other peak signals correspond to species with a mass of 344, 360 and 374 Dalton.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

LISTING OF CERTAIN SEQUENCES

>CAB12544.1-CYP102A2 bifunctional P-450/NADPH-P450 reductase 1 [Bacillus subtilis
subsp. subtilis str. 168]
MKETSPIPQPKTFGPLGNLPLIDKDKPTLSLIKLAEEQGPIFQIHTPAGTTIVVSGHELVKEVCDEERFD
KSIEGALEKVRAFSGDGLFTSWTHEPNWRKAHNILMPTFSQRAMKDYHEKMVDIAVQLIQKWARLNPNEA
VDVPGDMTRLTLDTIGLCGFNYRFNSYYRETPHPFINSMVRALDEAMHQMQRLDVQDKLMVRTKRQFRYD
IQTMFSLVDSIIAERRANGDQDEKDLLARMLNVEDPETGEKLDDENIRFQIITFLIAGHETTSGLLSFAT
YFLLKHPDKLKKAYEEVDRVLTDAAPTYKQVLELTYIRMILNESLRLWPTAPAFSLYPKEDTVIGGKFPI
TTNDRISVLIPQLHRDRDAWGKDAEEFRPERFEHQDQVPHHAYKPFGNGQRACIGMQFALHEATLVLGMI
LKYFTLIDHENYELDIKQTLTLKPGDFHISVQSRHQEAIHADVQAAEKAAPDEQKEKTEAKGASVIGLNN
RPLLVLYGSDTGTAEGVARELADTASLHGVRTKTAPLNDRIGKLPKEGAVVIVTSSYNGKPPSNAGQFVQ
WLQEIKPGELEGVHYAVFGCGDHNWASTYQYVPRFIDEQLAEKGATRFSARGEGDVSGDFEGQLDEWKKS
MWADAIKAFGLELNENADKERSTLSLQFVRGLGESPLARSYEASHASIAENRELQSADSDRSTRHIEIAL
PPDVEYQEGDHLGVLPKNSQTNVSRILHRFGLKGTDQVTLSASGRSAGHLPLGRPVSLHDLLSYSVEVQE
AATRAQIRELASFTVCPPHRRELEELSAEGVYQEQILKKRISMLDLLEKYEACDMPFERFLELLRPLKPR
YYSISSSPRVNPRQASITVGVVRGPAWSGRGEYRGVASNDLAERQAGDDVVMFIRTPESRFQLPKDPETP
IIMVGPGTGVAPFRGFLQARDVLKREGKTLGEAHLYFGCRNDRDFIYRDELERFEKDGIVTVHTAFSRKE
GMPKTYVQHLMADQADTLISILDRGGRLYVCGDGSKMAPDVEAALQKAYQAVHGTGEQEAQNWLRHLQDT
GMYAKDVWAGI (SEQ ID NO: 1)
>CAB14658.1-CYP102A3 cytochrome P450 CYP102A3 [Bacillus subtilis subsp. subtilis
str. 168]
MKQASAIPQPKTYGPLKNLPHLEKEQLSQSLWRIADELGPIFRFDFPGVSSVFVSGHNLVAEVCDEKRFD
KNLGKGLQKVREFGGDGLFTSWTHEPNWQKAHRILLPSFSQKAMKGYHSMMLDIATQLIQKWSRLNPNEE
IDVADDMTRLTLDTIGLCGFNYRFNSFYRDSQHPFITSMLRALKEAMNQSKRLGLQDKMMVKTKLQFQKD
IEVMNSLVDRMIAERKANPDENIKDLLSLMLYAKDPVTGETLDDENIRYQIITFLIAGHETTSGLLSFAI
YCLLTHPEKLKKAQEEADRVLTDDTPEYKQIQQLKYIRMVLNETLRLYPTAPAFSLYAKEDTVLGGEYPI
SKGQPVTVLIPKLHRDQNAWGPDAEDFRPERFEDPSSIPHHAYKPFGNGQRACIGMQFALQEATMVLGLV
LKHFELINHTGYELKIKEALTIKPDDFKITVKPRKTAAINVQRKEQADIKAETKPKETKPKHGTPLLVLF
GSNLGTAEGIAGELAAQGRQMGFTAETAPLDDYIGKLPEEGAVVIVTASYNGAPPDNAAGFVEWLKELEE
GQLKGVSYAVFGCGNRSWASTYQRIPRLIDDMMKAKGASRLTAIGEGDAADDFESHRESWENRFWKETMD
AFDINEIAQKEDRPSLSITFLSEATETPVAKAYGAFEGIVLENRELQTAASTRSTRHIELEIPAGKTYKE
GDHIGILPKNSRELVQRVLSRFGLQSNHVIKVSGSAHMAHLPMDRPIKVVDLLSSYVELQEPASRLQLRE
LASYTVCPPHQKELEQLVSDDGIYKEQVLAKRLTMLDFLEDYPACEMPFERFLALLPSLKPRYYSISSSP
KVHANIVSMTVGVVKASAWSGRGEYRGVASNYLAELNTGDAAACFIRTPQSGFQMPNDPETPMIMVGPGT
GIAPFRGFIQARSVLKKEGSTLGEALLYFGCRRPDHDDLYREELDQAEQDGLVTIRRCYSRVENEPKGYV
QHLLKQDTQKLMTLIEKGAHIYVCGDGSQMAPDVERTLRLAYEAEKAASQEESAVWLQKLQDQRRYVKDV
WTGM (SEQ ID NO: 2)
>CAB14997.1-CYP107H1 cytochrome P450 for pimelic acid formation for biotin
biosynthesis [Bacillus subtilis subsp. subtilis str. 168]
MTIASSTASSEFLKNPYSFYDTLRAVHPIYKGSFLKYPGWYVTGYEETAAILKDARFKVRTPLPESSTKY
QDLSHVQNQMMLFQNQPDHRRLRTLASGAFTPRTTESYQPYIIETVHHLLDQVQGKKKMEVISDFAFPLA
SFVIANIIGVPEEDREQLKEWAASLIQTIDFTRSRKALTEGNIMAVQAMAYFKELIQKRKRHPQQDMISM
LLKGREKDKLTEEEAASTCILLAIAGHETTVNLISNSVLCLLQHPEQLLKLRENPDLIGTAVEECLRYES
PTQMTARVASEDIDICGVTIRQGEQVYLLLGAANRDPSIFTNPDVFDITRSPNPHLSFGHGHHVCLGSSL
ARLEAQIAINTLLQRMPSLNLADFEWRYRPLFGFRALEELPVTFE (SEQ ID NO: 3)
>CAB14615.1-CYP107J1 cytochrome P450 [Bacillus subtilis subsp. subtilis str. 168]
MSSKEKKSVTILTESQLSSRAFKDEAYEFYKELRKSQALYPLSLGALGKGWLISRYDDAIHLLKNEKLKK
NYENVFTAKEKRPALLKNEETLTKHMLNSDPPDHNRLRTLVQKAFTHRMILQLEDKIQHIADSLLDKVQP
NKFMNLVDDYAFPLPIIVISEMLGIPLEDRQKFRVWSQAIIDFSDAPERLQENDHLLGEFVEYLESLVRK
KRREPAGDLISALIQAESEGTQLSTEELYSMIMLLIVAGHETTVNLITNMTYALMCHHDQLEKLRQQPDL
MNSAIEEALRFHSPVELTTIRWTAEPFILHGQEIKRKDVIIISLASANRDEKIFPNADIFDIERKNNRHI
AFGHGNHFCLGAQLARLEAKIAISTLLRRCPNIQLKGEKKQMKWKGNFLMRALEELPISF (SEQ ID NO: 4)
>ABQ22962.1-CYP107K1 cytochrome P450 [Bacillus subtilis subsp. subtilis str. 168]
MQMEKLMFHPHGKEFHHNPFSVLGRFREEEPIHRFELKRFGATYPAWLITRYDDCMAFLKDNRITRDVKN
VMNQEQIKMLNVSEDIDFVSDHMLAKDTPDHTRLRSLVHQAFTPRTIENLRGSIEQIAEQLLDEMEKENK
ADIMKSFASPLPFIVISELMGIPKEDRSQFQIWTNAMVDTSEGNRELTNQALREFKDYIAKLIHDRRIKP
KDDLISKLVHAEENGSKLSEKELYSMLFLLVVAGLETTVNLLGSGTLALLQHKKECEKLKQQPEMIATAV
EELLRYTSPVVMMANRWAIEDFTYKGHSIKRGDMIFIGIGSANRDPNFFENPEILNINRSPNRHISFGFG
IHFCLGAPLARLEGHIAFKALLKRFPDIELAVAPDDIQWRKNVFLRGLESLPVSLSK (SEQ ID NO: 5)
>CAB13078.1-CYP109B1 cytochrome P450 CYP109B1, monooxygenase [Bacillus subtilis
subsp. subtilis str. 168]
MNVLNRRQALQRALLNGKNKQDAYHPFPWYESMRKDAPVSFDEENQVWSVFLYDDVKKVVGDKELFSSCM
PQQTSSIGNSIINMDPPKHTKIRSVVNKAFTPRVMKQWEPRIQEITDELIQKFQGRSEFDLVHDFSYPLP
VIVISELLGVPSAHMEQFKAWSDLLVSTPKDKSEEAEKAFLEERDKCEEELAAFFAGIIEEKRNKPEQDI
ISILVEAEETGEKLSGEELIPFCTLLLVAGNETTTNLISNAMYSILETPGVYEELRSHPELMPQAVEEAL
RFRAPAPVLRRIAKRDTEIGGHLIKEGDMVLAFVASANRDEAKFDRPHMFDIRRHPNPHIAFGHGIHFCL
GAPLARLEANIALTSLISAFPHMECVSITPIENSVIYGLKSFRVKM (SEQ ID NO: 6)
>CAB15511.1-CYP134A1 cyclo-L-leucyl-L-leucyl dipeptide oxidase, pulcheriminic
synthase [Bacillus subtilis subsp. subtilis str. 168]
MSQSIKLFSVLSDQFQNNPYAYFSQLREEDPVHYEESIDSYFISRYHDVRYILQHPDIFTTKSLVERAEP
VMRGPVLAQMHGKEHSAKRRIVVRSFIGDALDHLSPLIKQNAENLLAPYLERGKSDLVNDFGKTFAVCVT
MDMLGLDKRDHEKISEWHSGVADFITSISQSPEARAHSLWCSEQLSQYLMPVIKERRVNPGSDLISILCT
SEYEGMALSDKDILALILNVLLAATEPADKTLALMIYHLLNNPEQMNDVLADRSLVPRAIAETLRYKPPV
QLIPRQLSQDTVVGGMEIKKDTIVFCMIGAANRDPEAFEQPDVFNIHREDLGIKSAFSGAARHLAFGSGI
HNCVGAAFAKNEIEIVANIVLDKMRNIRLEEDFCYAESGLYTRGPVSLLVAFDGA (SEQ ID NO: 7)
>CAB12004.1-CYP152A1 fatty acid beta-hydroxylating cytochrome P450 [Bacillus subtilis
subsp. subtilis str. 168]
MNEQIPHDKSLDNSLTLLKEGYLFIKNRTERYNSDLFQARLLGKNFICMTGAEAAKVFYDTDRFQRQNAL
PKRVQKSLFGVNAIQGMDGSAHIHRKMLFLSLMTPPHQKRLAELMTEEWKAAVTRWEKADEVVLFEEAKE
ILCRVACYWAGVPLKETEVKERADDFIDMVDAFGAVGPRHWKGRRARPRAEEWIEVMIEDARAGLLKTTS
GTALHEMAFHTQEDGSQLDSRMAAIELINVLRPIVAISYFLVFSALALHEHPKYKEWLRSGNSREREMFV
QEVRRYYPFGPFLGALVKKDFVWNNCEFKKGTSVLLDLYGTNHDPRLWDHPDEFRPERFAEREENLFDMI
PQGGGHAEKGHRCPGEGITIEVMKASLDFLVHQIEYDVPEQSLHYSLARMPSLPESGFVMSGIRRKS
(SEQ ID NO: 8)

1-55. (canceled)

56. A method comprising a step of:
culturing a microbial cell in the presence of a medium comprising a carbon source, a fatty acid, and amino acid and in the presence of an enzyme that covalently attaches a fatty acid to an amino acid under conditions and for a time sufficient for an acyl amino acid composition to be made, wherein the microbial cell comprises a modification that modulates hydroxylation and/or methylation of a fatty acid portion of the acyl amino acid.

57. The method of claim 56, wherein the microbial cell comprises a modification in a gene encoding a peptide synthetase.

58. The method of claim 57, wherein the modification results in reduced or abrogated expression of the gene that encodes a peptide synthetase.

59. The method of claim 56, wherein the modification that modulates hydroxylation and/or methylation of a fatty acid portion of the acyl amino acid results in an increase in the number of hydroxyl and/or methyl groups of a fatty acid portion of the acyl amino acid, as compared to that when the modification is absent.

60. The method of claim 56, wherein the modification that modulates hydroxylation and/or methylation of a fatty acid portion of the acyl amino acid results in a reduction in the number of hydroxyl and/or methyl groups of a fatty acid portion of the acyl amino acid, as compared to that when the modification is absent.

61. The method of claim 56, wherein the modification that modulates hydroxylation and/or methylation of a fatty acid portion of the acyl amino acid is or comprises a modification in a gene that encodes a fatty acid modifying enzyme.

62. The method of claim 56, wherein the modification that modulates hydroxylation and/or methylation of a fatty acid portion of the acyl amino acid is or comprises a modification in one or more regulatory elements operably linked to a gene that encodes a fatty acid modifying enzyme.

63. The method of claim 61, wherein the fatty acid modifying enzyme is or comprises a fatty acid hydroxylase and/or a fatty acid methylase.

64. The method of claim 63, wherein the fatty acid modifying enzyme is or comprises a fatty acid hydroxylase.

65. The method of claim 64, wherein the fatty acid hydroxylase is encoded by a gene selected from the group consisting of bioI, Cyp107h, cyp107J1, cyp134A1, cyp109B1, cyp152A1, cyp 102A2, cyp 102A3, cyp107K1, and combinations thereof.

66. The method of claim 64, wherein the fatty acid hydroxylase hydroxylates a branched fatty acid.

67. The method of claim 64, wherein the fatty acid hydroxylase hydroxylates an unbranched fatty acid.

68. The method of claim 64, wherein the fatty acid hydroxylase hydroxylates a fatty acid selected from the group consisting of caproic acid, caprylic acid, lauric acid, and myristic acid.

69. The method claim 56, wherein the modification that modulates hydroxylation and/or methylation of a fatty acid portion of the acyl amino acid results in reduced or abrogated expression of a gene that encodes a fatty acid modifying enzyme.

70. The method of claim 56, wherein the modification that modulates hydroxylation and/or methylation of a fatty acid portion of the acyl amino acid results in increased expression of a gene that encodes a fatty acid modifying enzyme.

71. The method of claim 56, wherein the microbial cell is a Bacillus subtilis cell.

72. The method of claim 56, wherein the acyl amino acid is acyl glycinate.

73. The method of claim 56, wherein the acyl amino acid is acyl glutamate.

74. The method of claim 56, wherein the acyl amino acid is acyl sarconsinate.


SEQUENCE LISTING (22 sequences)

SEQ ID NO: 1 - protein, 1061 aa, Bacillus subtilis
MKETSPIPQPKTFGPLGNLPLIDKDKPTLSLIKLAEEQGPIFQIHTPAGTTIVVSGHELVKEVCDEERFDKSIEGALEKVRAFSGDGLFTSWTHEPNWRKAHNILMPTFSQRAMKDYHEKMVDIAVQLIQKWARLNPNEAVDVPGDMTRLTLDTIGLCGFNYRFNSYYRETPHPFINSMVRALDEAMHQMQRLDVQDKLMVRTKRQFRYDIQTMFSLVDSIIAERRANGDQDEKDLLARMLNVEDPETGEKLDDENIRFQIITFLIAGHETTSGLLSFATYFLLKHPDKLKKAYEEVDRVLTDAAPTYKQVLELTYIRMILNESLRLWPTAPAFSLYPKEDTVIGGKFPITTNDRISVLIPQLHRDRDAWGKDAEEFRPERFEHQDQVPHHAYKPFGNGQRACIGMQFALHEATLVLGMILKYFTLIDHENYELDIKQTLTLKPGDFHISVQSRHQEAIHADVQAAEKAAPDEQKEKTEAKGASVIGLNNRPLLVLYGSDTGTAEGVARELADTASLHGVRTKTAPLNDRIGKLPKEGAVVIVTSSYNGKPPSNAGQFVQWLQEIKPGELEGVHYAVFGCGDHNWASTYQYVPRFIDEQLAEKGATRFSARGEGDVSGDFEGQLDEWKKSMWADAIKAFGLELNENADKERSTLSLQFVRGLGESPLARSYEASHASIAENRELQSADSDRSTRHIEIALPPDVEYQEGDHLGVLPKNSQTNVSRILHRFGLKGTDQVTLSASGRSAGHLPLGRPVSLHDLLSYSVEVQEAATRAQIRELASFTVCPPHRRELEELSAEGVYQEQILKKRISMLDLLEKYEACDMPFERFLELLRPLKPRYYSISSSPRVNPRQASITVGVVRGPAWSGRGEYRGVASNDLAERQAGDDVVMFIRTPESRFQLPKDPETPIIMVGPGTGVAPFRGFLQARDVLKREGKTLGEAHLYFGCRNDRDFIYRDELERFEKDGIVTVHTAFSRKEGMPKTYVQHLMADQADTLISILDRGGRLYVCGDGSKMAPDVEAALQKAYQAVHGTGEQEAQNWLRHLQDTGMYAKDVWAGI

SEQ ID NO: 2 - protein, 1054 aa, Bacillus subtilis
MKQASAIPQPKTYGPLKNLPHLEKEQLSQSLWRIADELGPIFRFDFPGVSSVFVSGHNLVAEVCDEKRFDKNLGKGLQKVREFGGDGLFTSWTHEPNWQKAHRILLPSFSQKAMKGYHSMMLDIATQLIQKWSRLNPNEEIDVADDMTRLTLDTIGLCGFNYRFNSFYRDSQHPFITSMLRALKEAMNQSKRLGLQDKMMVKTKLQFQKDIEVMNSLVDRMIAERKANPDENIKDLLSLMLYAKDPVTGETLDDENIRYQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQADIKAETKPKETKPKHGTPLLVLFGSNLGTAEGIAGELAAQGRQMGFTAETAPLDDYIGKLPEEGAVVIVTASYNGAPPDNAAGFVEWLKELEEGQLKGVSYAVFGCGNRSWASTYQRIPRLIDDMMKAKGASRLTAIGEGDAADDFESHRESWENRFWKETMDAFDINEIAQKEDRPSLSITFLSEATETPVAKAYGAFEGIVLENRELQTAASTRSTRHIELEIPAGKTYKEGDHIGILPKNSRELVQRVLSRFGLQSNHVIKVSGSAHMAHLPMDRPIKVVDLLSSYVELQEPASRLQLRELASYTVCPPHQKELEQLVSDDGIYKEQVLAKRLTMLDFLEDYPACEMPFERFLALLPSLKPRYYSISSSPKVHANIVSMTVGVVKASAWSGRGEYRGVASNYLAELNTGDAAACFIRTPQSGFQMPNDPETPMIMVGPGTGIAPFRGFIQARSVLKKEGSTLGEALLYFGCRRPDHDDLYREELDQAEQDGLVTIRRCYSRVENEPKGYVQHLLKQDTQKLMTLIEKGAHIYVCGDGSQMAPDVERTLRLAYEAEKAASQEESAVWLQKLQDQRRYVKDVWTGM

SEQ ID NO: 3 - protein, 395 aa, Bacillus subtilis
MTIASSTASSEFLKNPYSFYDTLRAVHPIYKGSFLKYPGWYVTGYEETAAILKDARFKVRTPLPESSTKYQDLSHVQNQMMLFQNQPDHRRLRTLASGAFTPRTTESYQPYIIETVHHLLDQVQGKKKMEVISDFAFPLASFVIANIIGVPEEDREQLKEWAASLIQTIDFTRSRKALTEGNIMAVQAMAYFKELIQKRKRHPQQDMISMLLKGREKDKLTEEEAASTCILLAIAGHETTVNLISNSVLCLLQHPEQLLKLRENPDLIGTAVEECLRYESPTQMTARVASEDIDICGVTIRQGEQVYLLLGAANRDPSIFTNPDVFDITRSPNPHLSFGHGHHVCLGSSLARLEAQIAINTLLQRMPSLNLADFEWRYRPLFGFRALEELPVTFE

SEQ ID NO: 4 - protein, 410 aa, Bacillus subtilis
MSSKEKKSVTILTESQLSSRAFKDEAYEFYKELRKSQALYPLSLGALGKGWLISRYDDAIHLLKNEKLKKNYENVFTAKEKRPALLKNEETLTKHMLNSDPPDHNRLRTLVQKAFTHRMILQLEDKIQHIADSLLDKVQPNKFMNLVDDYAFPLPIIVISEMLGIPLEDRQKFRVWSQAIIDFSDAPERLQENDHLLGEFVEYLESLVRKKRREPAGDLISALIQAESEGTQLSTEELYSMIMLLIVAGHETTVNLITNMTYALMCHHDQLEKLRQQPDLMNSAIEEALRFHSPVELTTIRWTAEPFILHGQEIKRKDVIIISLASANRDEKIFPNADIFDIERKNNRHIAFGHGNHFCLGAQLARLEAKIAISTLLRRCPNIQLKGEKKQMKWKGNFLMRALEELPISF

SEQ ID NO: 5 - protein, 407 aa, Bacillus subtilis
MQMEKLMFHPHGKEFHHNPFSVLGRFREEEPIHRFELKRFGATYPAWLITRYDDCMAFLKDNRITRDVKNVMNQEQIKMLNVSEDIDFVSDHMLAKDTPDHTRLRSLVHQAFTPRTIENLRGSIEQIAEQLLDEMEKENKADIMKSFASPLPFIVISELMGIPKEDRSQFQIWTNAMVDTSEGNRELTNQALREFKDYIAKLIHDRRIKPKDDLISKLVHAEENGSKLSEKELYSMLFLLVVAGLETTVNLLGSGTLALLQHKKECEKLKQQPEMIATAVEELLRYTSPVVMMANRWAIEDFTYKGHSIKRGDMIFIGIGSANRDPNFFENPEILNINRSPNRHISFGFGIHFCLGAPLARLEGHIAFKALLKRFPDIELAVAPDDIQWRKNVFLRGLESLPVSLSK

SEQ ID NO: 6 - protein, 396 aa, Bacillus subtilis
MNVLNRRQALQRALLNGKNKQDAYHPFPWYESMRKDAPVSFDEENQVWSVFLYDDVKKVVGDKELFSSCMPQQTSSIGNSIINMDPPKHTKIRSVVNKAFTPRVMKQWEPRIQEITDELIQKFQGRSEFDLVHDFSYPLPVIVISELLGVPSAHMEQFKAWSDLLVSTPKDKSEEAEKAFLEERDKCEEELAAFFAGIIEEKRNKPEQDIISILVEAEETGEKLSGEELIPFCTLLLVAGNETTTNLISNAMYSILETPGVYEELRSHPELMPQAVEEALRFRAPAPVLRRIAKRDTEIGGHLIKEGDMVLAFVASANRDEAKFDRPHMFDIRRHPNPHIAFGHGIHFCLGAPLARLEANIALTSLISAFPHMECVSITPIENSVIYGLKSFRVKM

SEQ ID NO: 7 - protein, 405 aa, Bacillus subtilis
MSQSIKLFSVLSDQFQNNPYAYFSQLREEDPVHYEESIDSYFISRYHDVRYILQHPDIFTTKSLVERAEPVMRGPVLAQMHGKEHSAKRRIVVRSFIGDALDHLSPLIKQNAENLLAPYLERGKSDLVNDFGKTFAVCVTMDMLGLDKRDHEKISEWHSGVADFITSISQSPEARAHSLWCSEQLSQYLMPVIKERRVNPGSDLISILCTSEYEGMALSDKDILALILNVLLAATEPADKTLALMIYHLLNNPEQMNDVLADRSLVPRAIAETLRYKPPVQLIPRQLSQDTVVGGMEIKKDTIVFCMIGAANRDPEAFEQPDVFNIHREDLGIKSAFSGAARHLAFGSGIHNCVGAAFAKNEIEIVANIVLDKMRNIRLEEDFCYAESGLYTRGPVSLLVAFDGA

SEQ ID NO: 8 - protein, 417 aa, Bacillus subtilis
MNEQIPHDKSLDNSLTLLKEGYLFIKNRTERYNSDLFQARLLGKNFICMTGAEAAKVFYDTDRFQRQNALPKRVQKSLFGVNAIQGMDGSAHIHRKMLFLSLMTPPHQKRLAELMTEEWKAAVTRWEKADEVVLFEEAKEILCRVACYWAGVPLKETEVKERADDFIDMVDAFGAVGPRHWKGRRARPRAEEWIEVMIEDARAGLLKTTSGTALHEMAFHTQEDGSQLDSRMAAIELINVLRPIVAISYFLVFSALALHEHPKYKEWLRSGNSREREMFVQEVRRYYPFGPFLGALVKKDFVWNNCEFKKGTSVLLDLYGTNHDPRLWDHPDEFRPERFAEREENLFDMIPQGGGHAEKGHRCPGEGITIEVMKASLDFLVHQIEYDVPEQSLHYSLARMPSLPESGFVMSGIRRKS

SEQ ID NO: 9 - DNA, 25 bp, synthetic construct
  misc_feature 1..25: note = Description of Artificial Sequence: Synthetic primer
  source 1..25: mol_type = other DNA
aaactaaaaaagggtagcctaaaaa

SEQ ID NO: 10 - DNA, 27 bp, synthetic construct
  misc_feature 1..27: note = Description of Artificial Sequence: Synthetic primer
  source 1..27: mol_type = other DNA
aaagaagttttagctataggagattcc

SEQ ID NO: 11 - DNA, 47 bp, synthetic construct
  misc_feature 1..47: note = Description of Artificial Sequence: Synthetic primer
  source 1..47: mol_type = other DNA
gattgtactgagagtgcaccatatgcttgcattaagagaaatttaca

SEQ ID NO: 12 - DNA, 41 bp, synthetic construct
  misc_feature 1..41: note = Description of Artificial Sequence: Synthetic primer
  source 1..41: mol_type = other DNA
gcggtatttcacaccgcagggaatacaagtcttttaatcag

SEQ ID NO: 13 - DNA, 37 bp, synthetic construct
  misc_feature 1..37: note = Description of Artificial Sequence: Synthetic primer
  source 1..37: mol_type = other DNA
gcggtgtgaaataccgcacagatgcgtaaggagaaaa

SEQ ID NO: 14 - DNA, 39 bp, synthetic construct
  misc_feature 1..39: note = Description of Artificial Sequence: Synthetic primer
  source 1..39: mol_type = other DNA
atatggtgcactctcagtacaatctgctctgatgccgca

SEQ ID NO: 15 - DNA, 7851 bp, synthetic construct
  misc_feature 1..7851: note = Description of Artificial Sequence: Synthetic polynucleotide
  source 1..7851: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcttgcattaagagaaatttacagggtgcttcaaattgacggccggttttatatcagtattgacacaaataccggtgaaaaagagaaaacgtatattcaactgctgaaagaccagcatttcagggatctttctgttatcaggcgtgcttcctgtctatgtattgtggctgttaaataaaaaatttctcgggaaatatatccaagatcctcgtattaggatttgggtatattttcttaattttttattttttgcatatactttatattaaaaaaagtttttttcatataaacttataacagaagaaagaacaaagaggtgatatcagacagggcagacattctttgtgaacaaaaggaatgaatattcattccgtaaacgaatcggaggttgtcagattacatgatatccgcatccagcagtaaatacgacatgattatgaaagcgtcagtctcactttttacggaaaggggttttgacgctaccactattcctatgatagctgaacgtgctcatgtagggacaggaacgatctatcgttattttgacagcaaagaaacactcgttaacgtactgtttcaagaaagcatccagcgatttacggaaaaactgaagcaagacgtttcagaattgcctgtcagagaaggctttcaccacgtattttgctgtctcgttcagtttacgaaagagagcgactatgcgcttttttttcttgaaaccaaaaaagacgctcattacttaaatcatacaagcaaaaaaatgatagaaaatctgactcaaatgcttgatgactattttaataagggaaaagcggaaggcgtgattcgcagcctgccctctaatgtgttaattgcgattgtattaggggcgtttctcaagatatatcagctcgttcaaacaggtgatatagagatggacactgatttaattactgaattggaacaatgctgctgggacgccattaagcttcattcatcacaaaaataggaaagggagatgttaatgaaacaggcaagcgcaatacctcagcccaaaacatacggacctttaaaaaatcttccgcatctggaaaaagaacagctttctcaatccttatggcggatagctgatgaattgggaccgattttccgttttgattttccgggagtatccagtgtttttgtgtccggccacaatcttgtggctgaagtgtgtgatgaaaaacgctttgacaagaaccttggcaaaggcttgcaaaaggtgcgtgagttcgggggagatggcttatttacaagctggacgcacgaaccgaactggcaaaaagcccaccgcattttgctgccgagttttagtcaaaaagcgatgaaaggctatcattctatgatgctggatatcgcaacccagctgattcaaaagtggagccggttaaaccctaatgaagaaattgatgtagcggacgatatgacacgtctgacgcttgatacgattgggttatgcgggtttaactatcgattcaacagcttttaccgtgattcacagcatccgtttatcaccagtatgctccgtgccttaaaagaggcgatgaatcaatcgaaaagactgggcctgcaagataaaatgatggtgaaaacgaagctgcagttccaaaaggatatagaagtcatgaactccctggttgatagaatgatagcggagcgaaaggcgaatccggatgaaaacattaaggatctcttgtctctcatgctttatgccaaagatccagtaacgggtgaaacgctggatgacgaaaacattcgataccaaatcatcacatttttaattgctggacatgagacaacaagcgggttgctatcctttgcgatttattgtctgcttacacatccggaaaaactgaaaaaagctcaggaggaagcggatcgcgtgttaacggatgacacgcctgaatataaacaaatccagcagctcaaatacattcggatggttttaaatgaaaccctcagactgtatccaacagctccggctttttctctatatgcgaaggaggatactgttctaggcggggaatatccgatcagcaaagggcagccagtcactgttttaattccaaaactgcaccgggatcaaaacgcttggggaccggatgcggaagatttccgtccggaacggtttgaggatccttcaagtatccctcaccatgcgtataagccgtttggaaacggacagcgcgcttgtattggcatgcagtttgctcttcaagaagcgacaatggttctcggtcttgtattaaagcattttgaattgataaaccatactggctacgaactaaaaatcaaagaagcattaacgatcaagccggatgattttaaaattactgtgaaaccgcgaaaaacagcggcaatcaatgtacagagaaaagaacaggcagacatcaaagcagaaacaaagccaaaagaaaccaaacctaaacacggcacacctttacttgttctttttggttcaaatcttgggacagctgagggaatagccggtgaactggctgctcaaggccgccagatgggctttacagctgaaacggctccgcttgatgattatatcggcaagctccctgaagaaggggcagtcgtcattgtaacggcttcttataatggggcgccgcctgataatgctgccggatttgtagagtggctgaaagagcttgaggaaggccaattgaaaggtgtttcctatgcggtattcggctgcggaaaccggagctgggccagcacgtatcagcggattccccgcctgattgatgacatgatgaaagcaaagggggcatcgcgtttaacagcgattggggaaggtgacgccgccgatgattttgaaagccaccgcgagtcttgggaaaaccgcttctggaaggaaacgatggacgcatttgatattaacgaaatagcccagaaagaagacaggccttcattatcgattacttttctcagtgaagcgacggaaacgccggttgctaaagcatatggcgcgtttgaagggattgtgttagagaatcgagaactccagacagctgccagcacgcgttcaacccgccatattgaattggaaattccggctggtaaaacatataaagaaggcgatcatatcggaatcctgccaaagaacagcagggagcttgttcagcgggttctcagccgattcggtttgcagtccaatcatgtgataaaagtaagcggaagcgctcatatggctcatctgccgatggatcggccaatcaaagtagtggatttattgtcgtcctatgtagagctgcaggaaccggcatcaaggcttcagcttcgggagctggcctcttatacagtttgtccgccgcatcaaaaagagctggaacagctcgtttcagatgatggcatttacaaagagcaggtacttgcaaaacgtcttaccatgcttgattttttagaggattatcctgcttgcgaaatgccgtttgaacggtttttagcacttttgccatcactaaaaccgagatactattccatttcaagctcaccgaaagttcatgcaaatatcgtgagcatgacggtaggagttgtgaaagcctcagcatggagcggccgaggtgaataccggggtgtcgcctctaattatttagcagaattgaatacaggtgatgcagcagcttgcttcattcgtacgccgcagtccggatttcagatgccgaatgatcctgaaacgcctatgattatggtcgggccgggcacaggaattgcgccattcagaggctttattcaggcaagatcggttttgaagaaggaaggaagcacccttggtgaagcacttttatacttcggctgccgccgcccggaccatgacgacctttacagagaagagctggatcaagcggaacaggacggtttggtcacaatccgccgatgctactcgcgcgtcgaaaacgaaccaaaaggatatgtccagcacttgctcaagcaagatacgcagaaattgatgacactcattgaaaaaggggctcatatttacgtatgcggtgatggatcgcaaatggctcctgatgtagagagaactttgcgattggcatatgaagctgaaaaagcagcaagtcaggaagaatcagctgtatggctgcaaaagctgcaagatcaaagacgttatgtgaaagacgtttggacaggaatgtaaaatataaaatcccgccaatctgattggcgggattgctttgcatatgagaaaaccggcacgataatgaaatctactgaaaaggatgttatagggagtatcgcgccggccttattattcatatcggcatgcagaggcaaaagtttagttctttttaccttgtttttaaaaataaatagtctgaaagtcttgtttttgattttcgactcaggcttttggcattttgttctttcttattccttaagatcagtcatctgttgaagattgatgcttgaattgctgttccacatgcttacggtaatgaaagtcatgaatcagccgaatggtcggcctgattaacagcagcaaacttccgatcgcaaacagccatatccctgccgacattaaccggtcataaaaaaagaaaaaacttccaacgagaaacatagcaccgatgataaaatcgtttactgtataaagaaccttatatcgttttttgaaaaaaagctcatatcgtttcaactctttttggatgtcatgttcttcatttcctttcattatcatccctccattcaattttggcttacccttacgtaaacggcatgtaaacataagggctgccttgctgaaagaagacataatcaacgatcagaaactaaagtaaaaaagtgatctgtataggatctcttttttacgatttcatcgcctgaacaataagagcagtcaaagtttttgcccctttaggaaccaagtgaacaccatcaggggtaaaatattccggatgctgaagagcttctgtatgccagtcaactaacgtaacattttgatgtgcgtgggcctgttgctgcaaagattcatttaccttgctttcccattggcggggaacccgtgtattgacaagataaatatgagcttttgagaaagattgaagcagttgttcgatttggctgtttgtaaaatagccattggttccaagctcaatgatgacagccttgttcggctgattaaaagacttgtattccctgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 40 bp, synthetic construct
  misc_feature 1..40: note = Description of Artificial Sequence: Synthetic primer
  source 1..40: mol_type = other DNA
cgcaactgtccatactctgaatataaaatcccgccaatct

SEQ ID NO: 17 - DNA, 42 bp, synthetic construct
  misc_feature 1..42: note = Description of Artificial Sequence: Synthetic primer
  source 1..42: mol_type = other DNA
gaacatcgtcaaaaaaccctaacatctccctttcctattttt

SEQ ID NO: 18 - DNA, 41 bp, synthetic construct
  misc_feature 1..41: note = Description of Artificial Sequence: Synthetic primer
  source 1..41: mol_type = other DNA
gggttttttgacgatgttcttgaaactcaatgtcttttttt

SEQ ID NO: 19 - DNA, 44 bp, synthetic construct
  misc_feature 1..44: note = Description of Artificial Sequence: Synthetic primer
  source 1..44: mol_type = other DNA
cagagtatggacagttgcggatgtacttcagaaaagattagatg

SEQ ID NO: 20 - DNA, 6941 bp, synthetic construct
  misc_feature 1..6941: note = Description of Artificial Sequence: Synthetic polynucleotide
  source 1..6941: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcttgcattaagagaaatttacagggtgcttcaaattgacggccggttttatatcagtattgacacaaataccggtgaaaaagagaaaacgtatattcaactgctgaaagaccagcatttcagggatctttctgttatcaggcgtgcttcctgtctatgtattgtggctgttaaataaaaaatttctcgggaaatatatccaagatcctcgtattaggatttgggtatattttcttaattttttattttttgcatatactttatattaaaaaaagtttttttcatataaacttataacagaagaaagaacaaagaggtgatatcagacagggcagacattctttgtgaacaaaaggaatgaatattcattccgtaaacgaatcggaggttgtcagattacatgatatccgcatccagcagtaaatacgacatgattatgaaagcgtcagtctcactttttacggaaaggggttttgacgctaccactattcctatgatagctgaacgtgctcatgtagggacaggaacgatctatcgttattttgacagcaaagaaacactcgttaacgtactgtttcaagaaagcatccagcgatttacggaaaaactgaagcaagacgtttcagaattgcctgtcagagaaggctttcaccacgtattttgctgtctcgttcagtttacgaaagagagcgactatgcgcttttttttcttgaaaccaaaaaagacgctcattacttaaatcatacaagcaaaaaaatgatagaaaatctgactcaaatgcttgatgactattttaataagggaaaagcggaaggcgtgattcgcagcctgccctctaatgtgttaattgcgattgtattaggggcgtttctcaagatatatcagctcgttcaaacaggtgatatagagatggacactgatttaattactgaattggaacaatgctgctgggacgccattaagcttcattcatcacaaaaataggaaagggagatgttagggttttttgacgatgttcttgaaactcaatgtctttttttgtagaatcaatagaagtgtgtaattgttgatgggacaataaaaaaggagctgaaacacagtatgggaaaggtttatgtatttgatcatcctttaattcagcacaagctgacatatatacggaatgaaaatacaggtacgaaggattttagagagttagtagatgaagtggctacactcatggcatttgaaattacccgcgatcttcctctggaagaagtggatatcaatacaccggttcaggctgcgaaatcgaaagtcatctcagggaaaaaactcggagtggttcctatcctcagagcaggattgggaatggttgacggcattttaaagctgattcctgcggcaaaagtgggacatgtcggcctttaccgtgatccagaaaccttaaaacccgtggaatactatgtcaagcttccttctgatgtggaagagcgtgaattcatcgtggttgacccgatgctcgctacaggcggttccgcagttgaagccattcacagccttaaaaaacgcggtgcgaaaaatatccgtttcatgtgtcttgtagcagcgccggagggtgtggaagaattgcagaagcatcattcggacgttgatatttacattgcggcgctagatgaaaaattaaatgaaaaaggatatattgttccaggtctcggagatgcgggtgaccgcatgtttggaacaaaataaaaaatgaaatccccaaaagggggtttcatttttttatccagttttttgctattcggtgaatctgtatacaattataggtgaaaatgtgaacattctgggatccgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttaactacaagctttttagacatctaatcttttctgaagtacatccgcaactgtccatactctgaatataaaatcccgccaatctgattggcgggattgctttgcatatgagaaaaccggcacgataatgaaatctactgaaaaggatgttatagggagtatcgcgccggccttattattcatatcggcatgcagaggcaaaagtttagttctttttaccttgtttttaaaaataaatagtctgaaagtcttgtttttgattttcgactcaggcttttggcattttgttctttcttattccttaagatcagtcatctgttgaagattgatgcttgaattgctgttccacatgcttacggtaatgaaagtcatgaatcagccgaatggtcggcctgattaacagcagcaaacttccgatcgcaaacagccatatccctgccgacattaaccggtcataaaaaaagaaaaaacttccaacgagaaacatagcaccgatgataaaatcgtttactgtataaagaaccttatatcgttttttgaaaaaaagctcatatcgtttcaactctttttggatgtcatgttcttcatttcctttcattatcatccctccattcaattttggcttacccttacgtaaacggcatgtaaacataagggctgccttgctgaaagaagacataatcaacgatcagaaactaaagtaaaaaagtgatctgtataggatctcttttttacgatttcatcgcctgaacaataagagcagtcaaagtttttgcccctttaggaaccaagtgaacaccatcaggggtaaaatattccggatgctgaagagcttctgtatgccagtcaactaacgtaacattttgatgtgcgtgggcctgttgctgcaaagattcatttaccttgctttcccattggcggggaacccgtgtattgacaagataaatatgagcttttgagaaagattgaagcagttgttcgatttggctgtttgtaaaatagccattggttccaagctcaatgatgacagccttgttcggctgattaaaagacttgtattccctgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 21 - protein, 97 aa, synthetic construct
  VARIANT 10..18: note = X can be any amino acid or gap
  VARIANT 20..21: note = X can be any amino acid or gap
  VARIANT 19: note = P residue can be absent
  VARIANT 47..48: note = PC residues can be absent
  VARIANT 64: note = X can be any amino acid
  VARIANT 65: note = K residue can be absent
  VARIANT 73: note = D residue can be absent
  VARIANT 74: note = X can be any amino acid
  VARIANT 75..76: note = YR residues can be absent
LIVSPADNKXXXXXXXXXPXXYPSTAGNCVSTAGNQCIVMSTAGCKPCSAGFYRLIVMSTAGDXKLIVMFYWDXYRLIVMFYWGAPTHQGSACQRHM

SEQ ID NO: 22 - protein, 31 aa, synthetic construct
  VARIANT 4..5: note = KG residues can be absent
  VARIANT 23..25: note = YAG residues can be absent
LIVKGLIVFYLIVMSTGHYWVSYAGGGSTAC